(19) United States
(12) Patent Application Publication
(10) Pub. No.: US 2006/0018948 A1
(43) Pub. Date: Jan. 26, 2006

(54) Biodegradable implantable medical devices, methods and systems

(76) Inventors: Patrick E. Guire, Eden Prairie, MN (US); Kristin S. Taton, Little Canada, MN (US); Jie Wen, Eden Prairie, MN (US); David M. DeWitt, Minneapolis (MN); Robert W. Hergenrother, Eden Prairie, MN (US); Aron B. Anderson, Minnetonka, MN (US)

Correspondence Address: KARRIE WEAVER;Kagan Binder, PLLC, Suite 200, 221 Main Street North, Stillwater, MN, 55082, US

(21) Appl. No.: 11/165,993

(22) Filed: Jun. 24, 2005

Related U.S. Application Data
(60) Provisional application No. 60/583,171, filed on Jun. 24, 2004.

(51) Int. Cl. A61F 2/00

(52) U.S. Cl. 424/426

(57) ABSTRACT

The invention provides implantable intraluminal medical devices that are fabricated of biodegradable materials. The invention further provides methods of treatment utilizing the devices.

This application claims the benefit of U.S. Provisional Application Ser. No. 60/583,171, filed Jun. 24, 2004, entitled “BIODEGRADABLE MEDICAL DEVICE,” which application is incorporated herein by reference in its entirety.

### FIELD OF THE INVENTION

The invention relates to medical devices having a biodegradable component that are useful for effectively treating a treatment site within a patient's body, for example, treatment of intraluminal areas (such as intravascular areas) and other areas within the body.

### BACKGROUND OF THE INVENTION

Tubular organs and structures such as blood vessels are subject to narrowing or occlusion of the lumen. Such narrowing or occlusion can be caused by a variety of traumatic or organic disorders, and symptoms can range from mild irritation and discomfort to paralysis and death. Treatment is typically site-specific and varies with the nature and extent of the occlusion.

Life threatening stenoses are most commonly associated with the cardiovascular system and are often treated using percutaneous transluminal coronary angioplasty (PTCA). This process improves the narrowed portion of the lumen by expanding the vessel's diameter at the blockage site using a balloon catheter. However, three to six months after PTCA, approximately 30% to 40% of patients experience restenosis. Restenosis is thought to be initiated by injury to the arterial wall during PTCA. Restenosis primarily results from vascular smooth muscle cell proliferation and extracellular matrix secretion at the injured site. Restenosis is also a major problem in non-coronary artery disease including the carotid, femoral, iliac, and renal arteries.

Stenosis of non-vascular tubular structures is often caused by inflammation, neoplasm and/or benign intimal hyperplasia. In the case of esophageal and intestinal strictures, the obstruction can be surgically removed and the lumen repaired by anastomosis. The smaller transluminal spaces associated with ducts and vessels can also be repaired in this fashion; however, restenosis caused by intimal hyperplasia is common. Furthermore, dehiscence is also frequently associated with anastomosis requiring additional surgery, which can result in increased tissue damage, inflammation, and scar tissue development leading to restenosis.

Recent advances in biomedical engineering have lead to the development of stents (mechanical scaffolds) to prevent restenosis and maintain the patency of vessels in the body. Stents are typically advanced through the vasculature to the deployment site while in a contracted state where they are then expanded to engage the vessel walls and thereby establish a flowpath therethrough. There are two general types of stents: permanent and temporary.

Permanent stents are used where long-term structural support or restenosis prevention is required, or in cases where surgical removal of the implanted stent is impractical. Permanent stents are typically fabricated from metals such as 316 stainless steel, MP35N alloy, and superelastic Nitinol (nickel-titanium).

It has been found that continued exposure of a stent to blood can lead to undesirable thrombus formation, and the presence of a stent in a blood vessel can over time cause the blood vessel wall to weaken, which creates the potential for an arterial rupture and/or the formation of an aneurysm. A stent can also become overgrown by tissue to the point that its usefulness can be substantially diminished while its continued presence can cause a variety of problems or complications.

As a result of limitations of long-term stents, recent research has been directed to temporary stents. Temporary stents can be generally categorized as removable and absorbable. Removable stents are typically implanted in areas of the body easily accessed to remove the device (for example, urethra).

Temporary absorbable stents can be fabricated from a wide range of synthetic biocompatible polymers depending upon the physical qualities desired. Representative biocompatible polymers include polyanhydrides, polycarbonates, polyesters, polyorthoesters, polyphosphazenes, and polyphosphate esters.

One type of polymeric system for fabricating temporary absorbable stents includes polylactic acid (PLA) and copolymers of polylactic acid with glycolic acid (such copolymers are commonly referred to as PLGA polymers). These polymeric systems can be used to fabricate drug delivery matrices, such as drug-loaded microspheres. PLGA-containing microspheres, however, can present a number of disadvantages. For example, the ability to manipulate the release of an encapsulated protein is limited because for most proteins, diffusion in PLGA matrices is negligible. The release of proteins from PLGA, therefore, depends upon the diffusion via pores present in the matrix and on the degradation or dissolution time of the microsphere. Also, during degradation of the PLGA, a low pH is generated in the polymeric matrix. A low pH environment, in turn, can be deleterious for many proteins as well as tissues (for example, by causing or exacerbating inflammation of tissues).

Moreover, polymers such as polylactic acid, polylactic acid-glycolic acid copolymer, and polycaprolactone can have other disadvantages. Generally, biodegradable or bioabsorbable stents fabricated from these materials exhibit bulk erosion and are as a consequence prone to break up into large particles as the polymeric matrix breaks down. Such bulk erosion can cause the material to flake or otherwise come apart in particulate form. Should such large particles actually become dislodged before becoming completely degraded, they could be washed downstream and cause emboli.

### SUMMARY OF THE INVENTION

Generally, the invention provides implantable intraluminal medical devices fabricated from biodegradable or bioresorbable materials. In some aspects, the polymeric formulations of the invention biodegrade within a period that is acceptable for the desired application. In certain aspects, such as in vivo therapy, such degradation occurs in a period usually less than about one year, or less than about six months, three months, one month, fifteen days, five days, three days, or even one day, on exposure to a physiological solution with a pH between 6 and 8 having a temperature in the range of about 25° to about 37° C. In some embodiments, the polymeric formulation of the invention degrades in a period in the range of about an hour to several weeks, depending upon the desired application.

In its article aspects, the invention provides an implantable intraluminal medical device comprising a body member fabricated of a biodegradable amphiphilic block copolymer comprising hydrophilic blocks and hydrophobic blocks. The body member is fabricated at least in part by the biodegradable amphiphilic block copolymer. The biodegradable amphiphilic copolymer is formulated to provide mechanical properties to the device.

In some aspects, the polymeric formulations of the invention biodegrade within a period that is acceptable for the desired application. In certain aspects, such as in vivo therapy, such degradation occurs in a period usually less than about four years, or less than about three years, or less than about two years, or less than about one year, or less than about six months, three months, one month, fifteen days, five days, three days, or even one day, on exposure to a physiological solution with a pH between 6 and 8 having a temperature in the range of about 25° to about 37° C. In some embodiments, the polymeric formulation of the invention degrades in a period in the range of about an hour to several weeks, depending upon the desired application.

The polymeric material used to fabricate the body member can be selected from a range of degradable materials described herein that provide one or more of the following mechanical properties to the overall device: (1) mimics the tissue it is designed to replace in size, shape, and material consistency; (2) is unlikely to induce infection or trigger a foreign body response; (3) is a temporary prosthesis that takes on characteristics of the natural tissue as it degrades; and (4) is a biocompatible implant that has a smooth surface to minimize risk for thrombus formation and macrophage enzyme activity.

In some aspects, the body member does not include bioactive agent. Alternatively, one or more bioactive agents can be included in the body member, when it is desired to deliver bioactive agent from the body member itself.

In some aspects, the implantable intraluminal medical devices of the invention provide mechanical properties at the implantation site and maintain these mechanical properties until they are no longer needed. After this period of time has elapsed, the medical device is degraded to an extent that the properties are no longer provided by the medical device, and the device components can be absorbed and/or excreted by the body. In some embodiments, the implantable medical device slowly degrades and transfers stress at the appropriate rate to surrounding tissues as these tissues heal and can accommodate the stress once borne by the medical device. Some illustrative mechanical properties that can be provided according to the invention are discussed in detail herein.

In some aspects, the body member can further include one or more degradable polymeric coatings on a surface. Typically, but not always, a bioactive agent included in the body member is released during a period subsequent to release of the bioactive agent from the coating. Alternatively, when it is desired to release bioactive agent from the body member during a time period that at least overlaps with a portion of the period of release of bioactive agent from the coating, it can be desirable to select the bioactive agents and polymeric coating materials to allow diffusion of the bioactive agent from the body member and through the coating material. When included, a coating can be provided on the entire surface of the body member, or substantially the entire surface. In other aspects, a coating can be provided on a selected portion of the body member surface. For example, a coating can be provided on the external, or vessel-contacting surface only, when it is desired to deliver bioactive agent to the vessel wall, but not toward the internal lumen of the body member. In still further aspects, more than one bioactive agent can be included in the coating. In some embodiments, for example, more than one bioactive agent can be provided in the entire coating. In other embodiments, different bioactive agents can be provided at different portions of the body member surface, such that one or more selected bioactive agents are included at one portion, and different selected bioactive agent(s) can be included at other portions (such as extraluminal versus intraluminal, or portions defined along the length of the device, and the like). The inventive methods and devices provide essentially unlimited ability to selectively deliver bioactive agents from various portions of the device, including the device body, device surface, or portions of either or both of these, as desired.

A “coating” as described herein can include one or more “coated layers,” each coated layer including one or more coating components (such as polymeric components, and/or bioactive agent). When more than one coated layer is applied to the surface of a device, it is typically applied successively. For example, a coating is typically formed by dipping, spraying, or brushing a coating material on a device to form a layer, and then drying the coated layer. The process can be repeated to provide a coating having multiple coated layers, wherein at least one layer includes a bioactive agent. Typically (but not always), at least the coated layer located nearest the device surface includes bioactive agent. In some aspects, more than two coated layers can be present. Such other layers can be the same or different than the first coated layer and/or second coated layer. Optionally, topcoats and/or priming layers can be included the coatings, and these topcoats and/or priming layers can be provided with or without bioactive agent. The suitability of the coating for use with a particular medical device, and in turn, the suitability of the application technique, can be evaluated by those skilled in the art, given the present description.

The biodegradable polymeric material for fabrication of the body member can be selected from a number of polymer materials. In some aspects, the biodegradable polymer is an amphiphilic copolymer comprising hydrophilic blocks and hydrophobic blocks. Illustrative amphiphilic copolymers are composed of polyalkylene glycol blocks (hydrophilic) and aromatic polyester blocks (hydrophobic). In other aspects, the polymer material can be selected from materials that can be viewed (for purposes of discussion) as falling within two general groups. The first group can be thought of as polymers containing ester linkages, such as polyetherester copolymers, terephthalate esters with phosphorus-containing linkages, and segmented copolymers with differing ester linkages. A second group is composed of polycarbonate-containing random copolymers. In another aspect, copolymers and/or blends of any of the biodegradable polymers listed herein can be utilized. Optionally, the polymer material can include one or more bioactive agents, thereby providing a drug-delivery device. Other optional components of the device include a sheath and/or microparticles (which include fibrous elements and microspheres). Further optional additives to the polymer material, such as antioxidants, hydrophobic materials, hydrophilic materials, and the like, can also be included as desired.

In some aspects, the invention provides devices and methods for providing treatment (for example, of passageways within the body, such as vascular structures), wherein the devices include at least a component that is biodegradable and/or bioerodable. According to some aspects of the invention, the device is replaced, at least in part, by body tissues over time. In some aspects, any portions of the device that remain in the body (are not degraded and/or resorbed) do not cause significant adverse foreign body response.

In some method aspects, the invention provides methods of making a device for the controlled release of bioactive agent, the method comprising steps of providing a biodegradable amphiphilic block copolymer comprising hydrophilic blocks and hydrophobic blocks, and forming the copolymer into an implantable intraluminal medical device. In some aspects, the method further includes a step of allowing the device to remain in the patient for a selected period of time, wherein the device is configured to degrade upon implantation for a degradation period, and wherein bioactive agent is released in a controlled manner for a release period, the release period constituting at least a portion of the degradation period. Generally, the degradation period is longer than the bioactive agent release period. In some aspects, the release period comprises 50% or less of the degradation period. In some aspects, the degradation period is 3 years or less, or 2 years or less, or in the range of 0.5 to 2 years.

In further aspects, the invention provides methods for delivery of bioactive agent to intraluminal sites within a patient in a controlled manner, the method comprising steps of implanting a device in an intraluminal implantation site within a patient, the device comprising a body member fabricated of a biodegradable amphiphilic block copolymer comprising hydrophilic blocks and hydrophobic blocks. In some aspects, the method further includes a step of allowing the device to remain in the patient for a selected period of time, wherein the device is configured to degrade upon implantation for a degradation period. Optionally, the device can include bioactive agent. In these aspects, bioactive agent can released in a controlled manner for a release period, the release period constituting at least a portion of the degradation period. Generally, the degradation period is longer than the bioactive agent release period. In some aspects, the release period comprises 50% or less of the degradation period. In some aspects, the degradation period is 3 years or less, or 2 years or less, or in the range of 0.5 to 2 years.

In further aspects, the invention provides methods for delivery of bioactive agent to a patient in a controlled manner, the method comprising steps of providing an implantable intraluminal device to a patient, the device comprising a body member fabricated of a biodegradable amphiphilic block copolymer comprising hydrophilic blocks and hydrophobic blocks. In some aspects, the method includes a step of allowing the device to remain in the patient for a selected period of time, during which time the bioactive agent is released from the device in a controlled and/or predictable manner.

Generally speaking, the inventive bioactive agent delivery systems can provide a controlled release profile of bioactive agent from the biodegradable implantable devices. The release profile is the cumulative mass of bioactive agent released versus time. The time profile of the release of bioactive agent, including immediate release and subsequent, sustained release can be predictably controlled utilizing the inventive compositions and methods. In some aspects, the initial release of bioactive agent is controlled, thereby permitting more of the bioactive agent to remain available at later times for a more extended release duration. The shape of the release profile after an initial release can be controlled to be linear, logarithmic, or some more complex shape, depending upon the composition of the biodegradable polymer and bioactive agent(s) in the polymer. In some embodiments, additives can be included in the biodegradable composition to further control the release rate. In some aspects, the inventive biodegradable compositions maintain bioactive agent levels within a therapeutic and/or prophylactic range and ideally a relatively constant level for sustained time periods.

In use, a biodegradable implantable medical device (optionally including bioactive agent in the body member and/or in a coating on a surface) is positioned within the body at a treatment site. In one such application, a stent is placed into a body vessel after a procedure, such as angioplasty. The stent is left in position, and the biodegradable polymer is allowed to degrade. Upon placement of the stent, and thus exposure of the biodegradable polymer to physiological fluids, bioactive agent is released from the stent. Typically, an initial release of the bioactive agent is observed, and over time a sustained release of the bioactive agent is observed. As the biodegradable polymer degrades, bioactive agent continues to be released in a controlled manner, thereby providing a therapeutically effective amount of the bioactive agent over a treatment course to the treatment site. Once the desired functional treatment (such as maintenance of vessel patency) has been completed, the body member of the device degrades as well. Some aspects of the invention thus provide a completely degradable device.

These and other aspects and advantages will now be described in more detail.

### DETAILED DESCRIPTION OF THE INVENTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

The invention is directed to implantable medical devices fabricated from a biodegradable material. At least a portion of the device is biodegradable, and this portion is broken down gradually by the body after implantation. The inventive devices and methods provide improved biodegradable devices that exhibit controlled release of one or more bioactive agents. The term “biodegradable” and is art-recognized and includes polymers, compositions and formulations, such as those described herein, that degrade during use. Such use includes in vivo use (such as in vivo therapy) and in vitro use. In general, degradation attributable to biodegradability involves the degradation of a biodegradable polymer into its component subunits, or digestion (for example, by a biochemical process), of the polymer into smaller, non-polymeric subunits. In certain embodiments, biodegradation may occur by enzymatic mediation, degradation in the presence of water and/or other chemical species in the body, or both.

The present invention is directed to methods and apparatuses for effectively treating a treatment site within a patient's body, and in particular for treating passages within the body such as vascular sites. According to some embodiments of the invention, degradable stents are provided that can provide treatment to a site within the body for a desired period of time, after which at least a portion of the stent degrades. In some embodiments, the inventive methods and apparatuses can be utilized to deliver bioactive agent to a treatment site as well. Such methods and apparatuses in accordance with the present invention can advantageously be used to provide flexibility in treatment duration and type of bioactive agent delivered to the treatment site. In some particular aspects, the present invention has been developed for controllably providing one or more bioactive agents to a treatment site within the body for a desired treatment course.

The term “implantation site” refers to the site within a patient's body at which the implantable device is placed according to the invention. In turn, a “treatment site” includes the implantation site as well as the area of the body that is to receive treatment directly or indirectly from a device component. For example, when the device includes one or more bioactive agents, bioactive agent can migrate from the implantation site to areas surrounding the device itself, thereby treating a larger area than simply the implantation site.

The implantable intraluminal medical devices according to the invention are capable of maintaining patency of a lumen at an implantation site for a desired period of time. Thus, the inventive devices provide mechanical properties and strength once implanted in a patient for a selected period of time that corresponds to a treatment course. In order to be properly introduced and utilized, implantable stents of all sorts of types can be designed to accommodate needs for compression resistance, expansion force, and mechanical stability once the device is implanted and expanded at a treatment site. Stents are designed to be deployed and expanded in different ways. A stent can be designed to self-expand upon release from its delivery system, or it may require application of a radial force through the delivery system to expand the stent to the desired implanted diameter. Self-expanding stents are compressed prior to insertion into the delivery device and released by the practitioner when correctly positioned within the implantation site. After release, the stent self expands to a predetermined diameter and is held in place by the expansion force or other physical features of the device. On the other hand, stents that require mechanical expansion by the practitioner are commonly deployed by a balloon-type catheter. Once positioned within the implantation site, the stent is expanded in situ to a size sufficient to fill the lumen and thereby open the lumen at the implantation site. Various designs and other mechanisms for expansion of stents have also been developed and will not be discussed further herein.

The mechanical properties of the stents are impacted by the materials utilized to fabricate the stent. Two physical qualities of the biodegradable polymer (or polymers) used to fabricate the stent play important roles in defining the overall mechanical qualities of the stent. These intrinsic polymer properties are tensile strength and tensile modulus. Tensile strength is defined as the force per unit area at the breaking point of the polymer. It is the amount of force, usually expressed in pounds per square inch (psi), that a substrate can withstand before it breaks or fractures. The tensile modulus, also expressed in psi, is the force required to achieve one unit of strain. Tensile modulus is an expression of a substrate's stiffness, or resistance to stretching, and relates directly to a stent's self-expansion properties.

Two important physical properties for stents are compression resistance and expansion force (radial force). Compression resistance relates to the stent's ability to withstand the surrounding tissue's circumferential pressure. A stent with poor compression resistance will not be capable of maintaining patency of the lumen. High compression resistance allows the stent to maintain the body lumen open and resist occluding forces such as elastic recoil or the growth of thrombus from the vessel wall. According to the invention, the inventive stents exhibit adequate compression resistance to withstand circumferential pressure exerted by tissues surrounding the implantation site. Expansion force relates to the amount of force utilized to expand the stent upon implantation to contact tissue surfaces at the implantation site. The combination of compression resistance and expansion force are competing qualities that are considered when formulating the polymeric materials of the invention. In some aspects, the biodegradable devices in accordance with the invention are formulated and/or fabricated to provide sufficient compression resistance such that the device that remains opposed to a lumen wall once implanted.

In some aspects of the invention, the biodegradable materials selected to fabricate the stent provide desired mechanical properties and in vivo degradation rate. The biodegradable materials are selected to provide a stent having sufficient tensile strength to maintain lumen patency, and tensile modulus to provide a suitably stiff device. In some aspects, the invention provides biodegradable devices that are capable of retaining their initial expansion force and compression resistance for a period of four or more weeks after implantation, or 6 or more weeks, or 8 or more weeks, or 10 or more weeks, or 12 or more weeks, or 20 or more weeks, or 24 or more weeks.

Another feature of a stent that impacts the mechanical properties of the device is the configuration of the stent. For example, microparticles (for example, in the form of microspheres and/or fibrous elements) can be included to provide improved compression resistance (including tensile strength and tensile modulus). In some illustrative embodiments, polymeric material can be fabricated in the form of fibers that can provide such features to a stent. In some aspects, the polymeric material of the fibers can be non-degradable, such that the fibers remain at the implantation site after degradation of portions of the device. Alternatively, the polymeric material can be biodegradable, such that the fibers degrade after they have provided sufficient structure to the treatment site, and additional reinforcement is no longer needed or desired. The topography of the stent can also enhance performance of the device. For example, microparticles can be associated on or near the surface of the device to provide a cell-reactive surface. Such a cell-reactive surface can provide an acceptable or preferential surface for cells present at the implantation site, thereby facilitating tissue ingrowth that can, in turn, enhance anchoring of the stent. In another example, polymeric material described herein can be provided in a particular form (for example, polymeric fibers) that are subsequently physically manipulated (for example, woven) to provide a final device configuration that possesses enhanced mechanical properties.

Optimization of tensile strength and tensile modulus can be achieved by selecting the composition of the polymeric material (as more fully described herein) and its physical characteristics, such as thickness of the polymeric material. In some embodiments, a portion of the overall medical device can comprise a different component than another. These one or more portions can comprise components of different physical characteristics and/or different materials. For example, a portion of the device can be biodegradable, while another portion of the device can be fabricated from a non-degradable material, such that this second portion of the device remains in the body after degradation of the biodegradable portion. Alternatively, different portions of the device can degrade at different rates.

In further aspects, the invention provides implantable medical devices fabricated from a biodegradable material, wherein the medical device provides at least some mechanical support or mechanical properties at the implantation site. In these aspects, the body of the device does not provide all of the structural features typically provided by a vascular stent, such as the typical compression resistance, expansion force, or the like. According to these embodiments, the degradable material used to fabricate the device is selected to possess sufficient tensile strength and tensile modulus for the desired application.

Thus, in some aspects, the implantable medical devices are fabricated of a biodegradable polymer having a tensile strength (polymer raw material tensile strength) in the range of about 40,000 to about 120,000 psi, or about 60,000 to about 120,000 psi, or about 90,000 to about 95,000 psi. In some aspects, the tensile modulus of biodegradable polymer utilized for fabricating the device is in the range of about 400,000 to about 2,000,000 psi, or in the range of about 700,000 to about 1,200,000 psi. It is understood that these ranges are illustrative only, and that one of skill in the art, upon review of the present disclosure, can readily determine a desirable tensile strength and tensile modulus for a biodegradable polymer to be formed into a device of the invention.

In some aspects, the inventive devices possess a tensile modulus of about 6,000 psi, or 7,000 psi, or about 8,000 psi, or about 9,000 psi, or about 10,000 psi.

In some aspects, the implantable medical devices provide a sufficient compression resistance to withstand compression of the lumen at the implantation site. Put another way, the compression resistance is the radial resistance (or radial force) of the device to external compression. One of skill in the art, upon review of the present disclosure, can readily determine the compression resistance to be provided at a selected implantation site. In some aspects, the inventive devices provide a minimum expansion force of about 1.2N, when the device is a self-expanding device. In some aspects, the devices can possess a minimum compression resistance of 5 N. In some aspects, the devices can possess a minimum compression resistance in the range of about 0.1 to about 0.2 lbs/mm.

In some aspects, the inventive devices provide a minimum longitudinal flexibility sufficient to allow insertion of the device within the patient. The longitudinal flexibility can be stated as the pure bending moment of a device, and it is dependent upon the length of the device. In some aspects, the devices possess suitable longitudinal flexibility so that the device can flex with natural motion of the lumen (such as natural blood vessel motion). In some aspects, the devices possess suitable longitudinal flexibility for delivery of the device to the implantation site (for example, when access to the implantation site requires travel through tortuous vasculature). An illustrative method for determining the longitudinal flexibility of a device is described in the Examples herein. Generally, the smaller the value of the flexibility, the more flexible the device is. For example, for a non-vascular stent (such as a urinary stent), a suitable longitudinal flexibility can be about 1.1 pounds of force, while a suitable value for a 15 mm vascular stent can be about 2 to 2.5 pounds of force. In some aspects, the inventive devices provide a minimum longitudinal flexibility of about 0.5 pounds of force for most intraluminal applications.

Another feature of the inventive devices that can impact mechanical properties of the overall device is the wall thickness of the device. The inventive devices can be fabricated to possess a wall thickness in the range of about 0.005 to about 20 mm, or in the range of 0.005 to about 5 mm for stents.

For stents, the initial unexpanded inner diameter of the device can be in the range of about 1 mm to about 5 mm. The expanded inner diameter can be in the range of about 1 mm to about 20 mm. The stent can be expandable to about 100% to about 400% or more of the initial inner diameter. An exemplary coronary stent can have an initial inner diameter of about 2 mm, and an expanded inner diameter of about 4 mm, with stent wall thickness in the range of about 0.005 to about 0.1 mm. In some aspects, the biodegradable polymer utilized has sufficient tensile strength so that the device wall can be kept relatively thin while resisting restenosis from lumen wall forces.

The inventive devices and methods have particular application in the field of coronary angioplasty. As used herein, the terms “stent” and “prosthesis” are used interchangeably to some extent in describing the invention, insofar as the methods, apparatus, and structures of the invention can be utilized not only in connection with an expandable intraluminal vascular graft for expanding partially occluded segments of a vessel, duct, body passageway, or duct, such as within an organ, but can also be utilized for many other purposes as an expandable prosthesis for many other types of body passageways. For example, expandable prostheses can also be used for such purposes as (1) supportive graft placement within blocked arteries opened by transluminal recanalization, but which are likely to collapse in the absence of internal support; (2) similar use following catheter passage through mediastinal and other veins occluded by inoperable cancers; (3) reinforcement of catheter created intrahepatic communications between portal and hepatic veins in patients suffering from portal hypertension; (4) supportive graft placement of narrowing of the esophagus, the intestine, the ureters, the urethra, and the like; (5) intraluminally bypassing a defect such as an aneurysm or blockage within a vessel or organ; and (6) supportive graft reinforcement of reopened and previously obstructed bile ducts. Accordingly, use of the term “prosthesis” encompasses the foregoing usages within various types of body passageways, and the use of the terms “intraluminal graft” or “intraluminal medical device” encompasses use for expanding and/or maintaining patency of the lumen of a body passageway. Further, the term “body passageway” encompasses any lumen or duct within the body, such as those previously described, as well as any vein, artery, or blood vessel within the vascular system.

Other vascular applications include anastamosis devices, occlusion devices (for treatment of such disorders as aneurysms or occlusions of blood vessels). Other illustrative applications include treatment of septal defects and closure devices.

Other non-vascular applications include neurological (brain), gastrointestinal, duodenum, biliary ducts, cystic duct, hepatic duct, esophagus, urethra, lymphatic vessels, reproductive tracts, prostate, trachea, and respiratory (such as bronchial) ducts, and otological applications.

Other applications include shunts for various applications, including hydrocephalus, cerebro-spinal fluid shunts, urological applications, glaucoma drain shunts; ear/nose/throat (for example, ear drainage tubes); renal devices; and dialysis (for example, grafts), nerve regeneration conduits, abdominal aortic aneurysm grafts, vascular intervention devices, urinary dilators, circulatory support systems, angiographic catheters, transition sheaths and dilators, tympanostomy vent tubes.

The inventive medical devices and systems are particularly useful for those devices that will come in contact with aqueous systems, such as bodily fluids. Such devices are adapted to release bioactive agent in a prolonged and controlled manner, generally beginning with the initial contact between the device surface and its aqueous environment. It is important to note that the local delivery of combinations of bioactive agents may be utilized to treat a wide variety of conditions utilizing any number of medical devices, or to enhance the function and/or life of the device. Essentially, any type of medical device may be fabricated in some fashion with one or more bioactive agents that enhances treatment over use of the singular use of the device or bioactive agent.

According to the present invention, a device has been developed that can be used to treat any implantation site within the body in which it is desirable to provide a device that degrades (at least in part) during use. In some embodiments, the device is used to treat an implantation site within the body in which it is desirable to restore and maintain patency of the implantation site while permitting function of the implantation site. For example, in vascular applications, the device can restore and maintain patency of the vascular site treated with the device, thus permitting continued blood flow through the treatment site. In some embodiments, the inventive device further provides controlled release of one or more bioactive agents.

More specifically, the device of the invention includes at least a component that is biodegradable, such that the component is broken down gradually by the body after implantation. The biodegradable component comprises a polymeric material that is formulated to degrade within the body at a desired rate. Optionally, the polymeric material can include one or more bioactive agents. According to this aspect of the invention, the device can provide controlled release of the bioactive agent to a treatment site within the body. As described herein, controlled release at the treatment site can mean control both in dosage rate and total dosage.

To facilitate the discussion of the invention, use of the invention to treat a vascular site will be addressed. Vascular treatment is selected because the features of the invention, particularly relating to degradative properties and optional drug delivery capabilities can be clearly presented. Further, the ability to provide a temporary medical device that can provide superior qualities while reducing risks to the patient can be a significant advance in the field. Emphasis is given to treatment of a vascular site with a stent; however, other illustrative devices that can utilize the inventive concepts include, but are not limited to, intraluminal devices such as intravascular devices, for example, vascular filters (for example, emboli filters) or extravascular devices (for example, located within organs such as the brain, stomach, reproductive organs, or within nonvascular passages such as the esophagus, and the like.

When the inventive devices include bioactive agent, the devices can be described as providing release of bioactive agent over time, and this relationship can be plotted to establish a release profile (cumulative mass of bioactive agent released versus time). Typically, the bioactive agent release profile can be considered to include an initial release of the bioactive agent, and a release of the bioactive agent over time, and the distinction between these two can often be simply the amount of time. The initial release is that amount of bioactive agent released shortly after the device is implanted, and the release of bioactive agent over time includes a longer period of time (for example, the lifespan of the biodegradable composition).

In some cases, the initial release can be characterized as a “burst” release. For systems that provide a “burst release” of bioactive agent, an initial release of a significant amount of bioactive agent is observed within a relatively short period of time after an implantable device is provided within a patient. A typical burst release is a much higher release in a relatively short amount of time (for example, more than 30% of the amount of bioactive agent contained in the device within the first 24 hours after implantation). In contrast, inventive devices can provide substantially linear release of bioactive agent, wherein the initial release of bioactive agent does not comprise a significantly different slope or shape than the overall release profile. Put another way, a burst release can be characterized as an initial release that differs in magnitude of bioactive agent released (that is, a significant amount is released during the initial period).

The significance of a burst release can also be considered in relation to the particular polymeric material that contains the bioactive agent. For example, for a biodegradable polymer having a half-weight degradation time of four weeks, a significant burst release can be considered to be more than about 30% of the bioactive agent contained in the biodegradable polymer that is released within the first 24-hour period. For a biodegradable polymer having a half-weight degradation time of more than four weeks, a longer burst time period can be considered significant for the same amount of bioactive agent. For example, the half-weight degradation time of PLA is 155 days compared to 30 days for PLGA. Thus, a longer time period would be considered therapeutically relevant for the burst release from PLA compared to PLGA.

In accordance with some aspects of the invention, the shape of the bioactive agent release curve can be modulated by controlling one or more characteristics of the bioactive agent delivery systems, such as the selection of the polymer materials, the relative amounts of polymer components within the system (for example, when the system comprises a blend of more than one polymer material), and the like. In accordance with the invention, the time profile of the release of bioactive agent can be modulated to provide any desired shape, including immediate release where the bioactive agent elutes all at once (much like a step function) to an extremely slow, linear (i.e., zero order) release, where the bioactive agent is evenly released over many months or years. Depending on the bioactive agent and the condition being treated, a variety of release profiles can be achieved. The objective of creating bioactive agent delivery systems of the inventive devices is to be able to attain the broad range of release profiles that lie between a step function and a low-slope, zero-order release. In some aspects, the polymer materials selected (and the relative amounts of polymers, when more than one polymer material is included in the system) of the bioactive agent delivery system is selected to provide the desired release profile. By controlling the release profiles as described herein, significant improvements can be made to the efficacy of treatment with bioactive agent.

The inventive bioactive agent delivery systems described herein can be designed to control (such as, for example, by limiting or even eliminating) the initial burst of bioactive agent from the biodegradable polymer. The bioactive agent still remaining in the biodegradable polymer after the burst release is then released to the site of action over a longer time period. The shape of the release profile (percentage of bioactive agent released versus time) after the burst can be controlled to be linear or logarithmic or some more complex shape, again depending upon the composition of the biodegradable polymer and bioactive agent in the biodegradable polymer.

The in vivo release of a bioactive agent can be approximated by observing the in vitro release of the bioactive agent. For example, an implantable device can be fabricated to include a biodegradable polymer containing a bioactive agent. The implantable device can then be placed in an appropriate solution (for example, a buffer solution such as phosphate buffered saline) for a period of time. During incubation of the device, the solution can be periodically monitored to determine the in vitro release rate of the bioactive agent into the solution. The implantable device is removed from the solution and placed in fresh buffer solution in a new vial at periodic sampling times. Concentration of bioactive agent at each sampling time can be determined in the spent buffer by spectroscopy using the characteristic wavelength for each bioactive agent. The concentration can be converted to a mass of bioactive agent released from the device using molar absorptivities. The cumulative mass of the released bioactive agent can be calculated by adding the individual sample mass at each sampling time. The release profile can be obtained by plotting the cumulative mass of released bioactive agent as a function of time. From this determined in vitro release rate, the in vivo release rate can be approximated using known techniques. Typically, the in vitro release rate is slower than an in vivo release rate for the same bioactive agent and biodegradable composition.

The inventive biodegradable compositions exhibit controlled release characteristics, in contrast to a bolus type administration (which includes an initial burst release of bioactive agent) in which a substantial amount of the bioactive agent is made biologically available at one time. For example, in some embodiments, upon contact with body fluids including blood, spinal fluid, lymph, or the like, the biodegradable compositions (formulated as provided herein) can permit a desired amount of initial release of bioactive agent, and subsequently provide a sustained, predictable delivery of the bioactive agent over time. This release can result in prolonged delivery of therapeutically effective amounts of any incorporated bioactive agent. Sustained release will vary in certain embodiments as described in more detail herein.

The phrase “therapeutically effective amount” is an art-recognized term. In some aspects, the term refers to an amount of the bioactive agent that, when incorporated into a biodegradable composition of the invention, produces some desired effect at a reasonable benefit/risk ratio applicable to any medical treatment. In some aspects, the term refers to that amount necessary or sufficient to eliminate or reduce risk of restenosis. The phase “prophylactically effective amount” likewise is an art-recognized term. In some aspects, the phrase refers to an amount of bioactive agent that, when incorporated into a biodegradable composition of the invention, provides a preventative effect sufficient to prevent or protect an individual from future medical risk associated with a particular disease or disorder. The therapeutically and/or prophylactically effective amount can vary depending upon such factors as the condition being treated (or to be prevented), the particular bioactive agent(s) being administered, the size of the patient, the severity of the condition, and the like. In some aspects, the therapeutically and/or prophylactically effective amount takes into account the amount of bioactive agent released from the biodegradable composition during any selected time period, particularly the time period during implantation and immediately after the device is emplaced (the initial release). Thus, the therapeutically and/or prophylactically effective amount also applies to the initial release of bioactive agent from the biodegradable composition. By controlling the initial release from the biodegradable composition, some embodiments can reduce or eliminate potentially undesirably high amounts of bioactive agent release during early stages after implantation. One of ordinary skill in the art can empirically determine the effective amount of a particular bioactive agent without necessitating undue experimentation.

Aspects of the invention can thus provide one or more advantages, including the ability to provide sustained bioactive agent delivery that can maintain the bioactive agent concentration within a therapeutic window for a prolonged period of time and improve bioactive agent efficacy. Local delivery can reduce bioactive agent dosage, toxicity effects, and other side effects that are typically associated with administration of therapeutics.

Biodegradable Polymeric Materials

For purposes of describing the invention, use of polyethylene glycol terephthalate/polybutylene terephthalate copolymer (PEGT/PBT) as a biodegradable polymeric material is specifically addressed. However, one of skill in the art, upon review of this disclosure, will readily appreciate that the features of the PEGT/PBT polymer system apply to the additional polymer systems described herein as well.

As used herein, the term “aliphatic” refers to a linear, branched, or cyclic alkane, alkene, or alkyne. Illustrative aliphatic groups in polymeric materials that include phosphoester linkages are linear or branched alkanes having 1 to 10 carbon atoms, or linear alkane groups having 1 to 7 carbon atoms.

As used herein, the term “aromatic” refers to an unsaturated cyclic carbon-containing compound with 4n+2 π electrons.

As used herein, the term “heterocyclic” refers to a saturated or unsaturated ring compound having one or more atoms other than carbon in the ring, for example, nitrogen, oxygen or sulfur.

In accordance with one aspect of the invention, medical devices are described for treatment of vascular structures, such as stents, the devices including at least a component that is biodegradable. The biodegradable material is selected from particular degradable polymers containing ester linkages (polyetherester copolymers, terephthalate esters with phosphorus-containing linkages, and segmented copolymers with differing ester linkages); or polycarbonate-containing random copolymers; or copolymers and/or blends of any of these. Each of these polymeric biodegradable materials will be described in detail.

In some embodiments, the polyetherester copolymers are amphiphilic block copolymers that include hydrophilic (for example, a polyalkylene glycol, such as polyethylene glycol) and hydrophobic blocks (for example, polyethylene terephthalate).

In one embodiment, the polyetherester copolymer comprises a first component that is a polyalkylene glycol, and a second component which is a polyester formed from an alkylene glycol having from 2 to 8 carbon atoms and a dicarboxylic acid. The polyalkylene glycol, in one embodiment, is selected from the group consisting of polyethylene glycol, polypropylene glycol, and polybutylene glycol. In one embodiment, the polyalkylene glycol is polyethylene glycol.

In another embodiment, the polyester is selected from the group consisting of polyethylene terephthalate, polypropylene terephthalate, and polybutylene terephthalate. In a particular embodiment, the polyester is polybutylene terephthalate.

In a particular embodiment, the copolymer is a polyethylene glycol/polybutylene terephthalate block copolymer.

In another embodiment, the polyester has the following structural formula I: 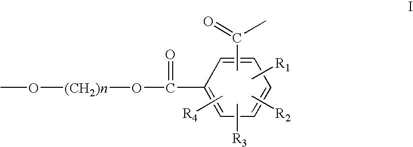
wherein n is from 2 to 8, and each of R₁, R₂, R₃, and R₄ is hydrogen, halogen (such as chlorine, iodine, bromine), nitro-, or alkoxy, and each of R₁, R₂, R₃ and R₄ is the same or different. In one particular embodiment, each of R₁, R₂, R₃ and R₄ are hydrogen. Alternatively, the ester is derived from a binuclear aromatic diacid having the following structural formula II: 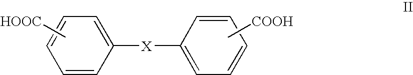
wherein X is —O—, —SO₂—, or —CH₂—.

In one embodiment, the copolymer is a segmented thermoplastic biodegradable polymer comprising a plurality of recurring units of the first component and units of the second component. The first component comprises about 30 weight percent to about 99 weight percent (based upon the weight of the copolymer) of units of the formula III:
—OLO—CO—R—CO— III
wherein L is a divalent organic radical remaining after removal of terminal hydroxyl groups from a poly(oxyalkylene)glycol, O represents oxygen, C represents carbon, and R is a substituted or unsubstituted divalent radical remaining after removal of carboxyl groups from a dicarboxylic acid.

The second component is present in an amount of about 1 weight percent to about 70 weight percent (based upon the weight of the copolymer), and is comprised of units of the formula IV:
—OEO—CO—R—CO— IV
wherein E is an organic radical selected from the group consisting of a substituted or unsubstituted alkylene radical having from 2 to 8 carbon atoms, and a substituted or unsubstituted ether moiety. R is a substituted or unsubstituted divalent aromatic radical.

The poly(oxyalkylene)glycol, in one embodiment, can be selected from the group consisting of poly(oxyethylene)glycol, poly(oxypropylene)glycol, poly(oxybutylene)glycol, and combinations of any one or more of these. In some embodiments, the poly(oxyalkylene)glycol is poly(oxyethylene)glycol.

The poly(oxyethylene)glycol can have a molecular weight in the range of about 200 to about 20,000, or about 200 to about 10,000. The precise molecular weight of the poly(oxyethylene)glycol is dependent upon a variety of factors, including the type of bioactive agent (if any) incorporated into the polymeric matrix.

In one embodiment, E is a radical selected from the group consisting of a substituted or unsubstituted alkylene radical having from 2 to 8 carbon atoms, or having from 2 to 4 carbon atoms. In some embodiments, the second component is selected from the group consisting of polyethylene terephthalate, polypropylene terephthalate, and polybutylene terephthalate. In one embodiment, the second component is polybutylene terephthalate.

In a particular embodiment, the copolymer is a polyethylene glycol/polybutylene terephthalate copolymer.

In one embodiment, the polyethylene glycol/polybutylene terephthalate copolymer can be synthesized from a mixture of dimethylterephthalate, butanediol (in excess), polyethylene glycol, an antioxidant, and catalyst. The mixture is placed in a reaction vessel and heated to about 180° C., and methanol is distilled as transesterification occurs. During the transesterification, the ester bond with methyl is replaced with an ester bond with butylene and/or the polyethylene glycol. In this step, the polyethylene glycol does not react. After transesterification, the temperature is raised slowly to about 245° C., and a vacuum (finally less than 0.1 mbar) is achieved. The excess butanediol is distilled and a prepolymer of butanediol terephthalate condenses with the polyethylene glycol to form a polyethylene glycol/polybutylene terephthalate copolymer. A terephthalate moiety connects the polyethylene glycol units to the polybutylene terephthalate units of the copolymer, and this copolymer is sometimes hereinafter referred to as a polyethylene glycol terephthalate/polybutylene terephthalate copolymer (also referred to as PEGT/PBT or PEG/PBT copolymer). Alternatively, the polyethylene glycol is present as free polyethylene glycol that is mixed with PEGT/PBT copolymer. In another alternative, polyalkylene glycol/polyester copolymers can be prepared as described in U.S. Pat. No. 3,908,201.

The PEGT/PBT copolymer can also be obtained from OctoPlus BV, Bilthoven, The Netherlands, under the product name PolyActive™.

The above discussion of illustrative copolymers is not intended to limit the invention to the specific copolymers discussed, or to any particular synthesis means thereof.

The polymeric matrix can be formulated to provide desired degradation rates. Degradation of the polymeric matrix occurs by hydrolysis of the ester linkages, and/or oxidation of ether groups. Further, when the polymeric matrix includes a bioactive agent, the formulation of the polymeric matrix can be adjusted to control the rate of diffusion of the bioactive agent from the polymer when desired.

In some embodiments, the degradation rate of PEGT/PBT copolymer can be controlled in two general manners. For example, the degradation rate can be increased by including 22 hydrophilic antioxidants in the polymeric material. In addition, or alternatively, the degradation rate can be increased by partially replacing the aromatic groups with aliphatic groups. For example, the more hydrophobic aromatic groups, such as terephthalate groups, can be replaced with less hydrophobic groups, such as diacid groups (for example, succinate). In another example, more hydrophobic butylene groups can be at least partially replaced with less hydrophobic groups, such as dioxyethylene. The degree of replacement can be determined to provide a selected affect on degradation rate.

In accordance with the invention, an increased degradation of the polyetherester copolymer is not accompanied by a significant, deleterious increase in acid formation. Degradation of the copolymer takes place by hydrolysis of ester linkages and oxidation of ether groups, which can generate a certain amount of acid. However, the levels of acid generated during degradation are, in one aspect, lesser than the levels generated by other known degradable polymers (such as PLA), and in another aspect, are not deleterious to tissues and/or bioactive agent. The acidity of the degradation environment can impact the stability of bioactive agents in that environment. Optionally, hydrophilic antioxidants can be included in the polymer material. Such hydrophilic antioxidants will be described in more detail elsewhere herein and can be particularly desirable when the polymeric matrix includes peptide or protein molecules. According to this aspect of the invention, when the protein or peptide molecule is released from the polymeric matrix upon degradation thereof, the protein is not denatured by acid degradation products. This can provide significant advantages over degradable polymers that include PLA or PLGA, where degradation increases acidity of the polymeric environment. These aspects of the invention will be described in more detail with respect to embodiments of the invention where bioactive agents are released from the polymeric matrix.

In some embodiments of the invention, the polymeric material comprises a biodegradable terephthalate copolymer that includes a phosphorus-containing linkage. Polymers having phosphoester linkages, called poly(phosphates), poly(phosphonates) and poly(phosphites), are known. See, for example, Penczek et al., Handbook of Polymer Synthesis, Chapter 17: “Phosphorus-Containing Polymers,” 1077-1132 (Hans R. Kricheldorf ed., 1992), as well as U.S. Pat. Nos. 6,153,212, 6,485,737, 6,322,797, 6,600,010, 6,419,709. The respective structures of each of these three classes of compounds, each having a different side chain connected to the phosphorus atom, is as follows:

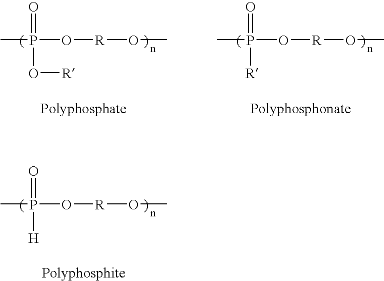

The versatility of these polymers is related to the versatility of the phosphorus atom, which is known for a multiplicity of reactions. Its bonding can involve the 3p orbitals or various 3s-3p hybrids; spd hybrids are also possible because of the accessible d orbitals. Thus, the physicochemical properties of the poly(phosphoesters) can be readily changed by varying either the R or R′ group. The biodegradability of the polymeric material according to these embodiments is related to the physiologically labile phosphoester bond in the backbone of the polymer. By manipulating the backbone or the side chain, a wide range of biodegradation rates are attainable.

An additional feature of the poly(phosphoesters) is the availability of functional side groups. Because phosphorus can be pentavalent, bioactive agents (such as drugs) can be chemically linked to the polymer. For example, bioactive agents with carboxyl groups can be coupled to the phosphorus via an ester bond, which is hydrolyzable. The P—O—C group in the backbone also lowers the glass transition temperature (Tg) of the polymer and, importantly, confers solubility in common organic solvents, which can be desirable for characterization and processing of the polymer.

In one embodiment, the terephthalate polyester includes a phosphoester linkage that is a phosphite. Suitable terephthalate polyester-polyphosphite copolymers are described, for example, in U.S. Pat. No. 6,419,709 (Mao et al., “Biodegradable Terephthalate Polyester-Poly(Phosphite) Compositions, Articles, and Methods of Using the Same”). According to this embodiment, the polymeric material comprises recurring monomeric units of the following formula V: 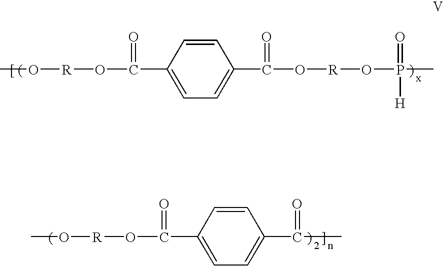
wherein R is a divalent organic moiety. R can be any divalent organic moiety so long as it does not interfere with the polymerization, copolymerization, or biodegradation reactions of the copolymer. Specifically, R can be an aliphatic group, for example, alkylene, such as ethylene, 1,2-dimethylethylene, n-propylene, isopropylene, 2-methylpropylene, 2,2-dimethylpropylene or tert-butylene, tert-pentylene, n-hexylene, n-heptylene, and the like; alkenylene, such as ethenylene, propenylene, dodecenylene, and the like; alkynylene, such as propynylene, hexynylene, octadecynylene, and the like; an aliphatic group substituted with a non-interfering substituent, for example, hydroxy-, halogen-, or nitrogen-substituted aliphatic group; or a cycloaliphatic group such as cyclopentylene, 2-methylcyclopentylene, cyclohexylene, and the like.

R can also be a divalent aromatic group, such as phenylene, benzylene, naphthalene, phenanthrenylene, and the like, or a divalent aromatic group substituted with a non-interfering substituent. Further, R can also be a divalent heterocyclic group, such as pyrrolylene, furanylene, thiophenylene, alkylene-pyrrolylene-alkylene, pyridylene, pyridinylene, pyrimidinylene, and the like; or can be any of these substituted with a non-interfering substituent.

In some embodiments, R is an alkylene group, a cycloaliphatic group, a phenylene group, or a divalent group having the formula VI: 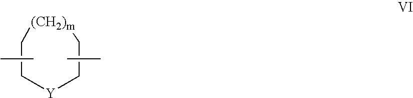
wherein Y is oxygen, nitrogen, or sulfur, and m is 1 to 3. In some embodiments, R is an alkylene group having 1 to 7 carbon atoms and, in some aspects, R is an ethylene group.

The value of x can vary depending upon the desired solubility of the polymer, the desired Tg, the desired stability of the polymer, the desired stiffness of the final polymers, and the biodegradability and release characteristics desired in the polymer. In general, x is 1 or more, and typically, x varies between 1 and 40. In some embodiments, x is in the range of 1 to 30, or in the range of 1 to 20, or in the range of 2 to 20.

The number n can vary greatly depending upon the biodegradability and the release characteristics desired in the polymer, but typically varies in the range of about 3 to about 7,500, or about 5 to about 5,000. In some embodiments, n is in the range of about 5 to about 300, or in the range of about 5 to about 200.

The most common general reaction in preparing a poly(phosphite) is a condensation of a diol with a dialkyl or diaryl phosphite according to the following equation:

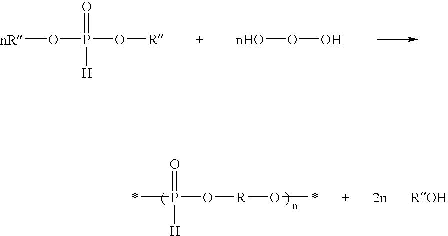

Poly(phosphites) can also be obtained by employing tetraalkyldiamides of phosphorus acid as condensing agents, according to the following equation:

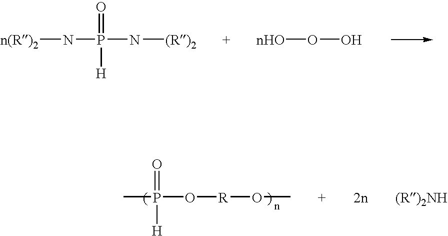

The above polymerization reactions can be either in bulk or solution polymerization. An advantage of bulk polycondensation is that it avoids the use of solvents and large amounts of other additives, thus making purification more straightforward. It can also provide polymers of reasonably high molecular weight.

Typical solvents for solution polycondensation include chlorinated organic solvents, such as chloroform, dichloromethane, or dichloroethane. In some embodiments, the solution polymerization is run in the presence of equimolar amounts of the reactants and a stoichiometric amount of an acid acceptor, usually a tertiary amine such as pyridine or triethylamine. The product is then typically isolated from the solution by precipitation with a nonsolvent and purified to remove the hydrochloride salt by conventional techniques known to those of ordinary skill in the art, such as by washing with an aqueous acidic solution, such as dilute hydrochloric acid.

Interfacial polycondensation can be used when high molecular weight polymers are desired at high reaction rates. Mild conditions minimize side reactions. Also, the dependence of high molecular weight on stoichiometric equivalence between diol and phosphite inherent in solution methods is removed. However, hydrolysis of the acid chloride may occur in the alkaline aqueous phase. Phase transfer catalysts, such as crown ethers or tertiary ammonium chloride, can be used to bring the ionized diol to the interface to facilitate the polycondensation reaction. The yield and molecular weight of the resulting polymer after interfacial polycondensation can be affected by reaction time, molar ratio of the monomers, volume ratio of the immiscible solvents, the type of acid acceptor, and the type and concentration of the phase transfer catalyst.

In one embodiment, the process of making the biodegradable terephthalate polymer of formula V comprises the steps of polymerizing p moles of a diol compound having formula VIII: 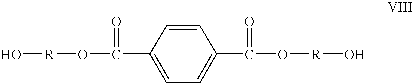
wherein R is as defined above for formula VI, with q moles of dialkyl or diaryl of formula IX: 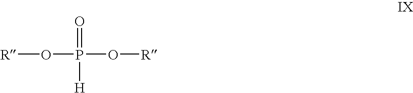
wherein p>q, to form q moles of a homopolymer of formula X, shown below: 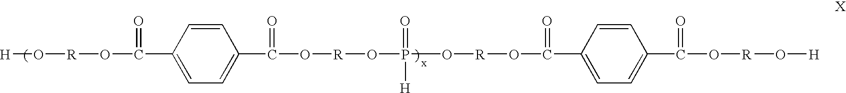
wherein R and x are as defined above for formulae V and VIII. The homopolymer so formed can be isolated, purified and used as is. Alternatively, the homopolymer, isolated or not, can be used to prepare a block copolymer composition of the invention by the steps of: (a) polymerizing as described above, and (b) further reaction the homopolymer of formula X with (p-q) moles of terephthaloyl chloride having the formula XI: 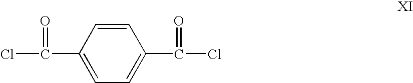
to form the copolymer of formula V.

The polymerization step (a) can take place at widely varying temperatures, depending upon the solvent used, the solubility desired, the molecular weight desired, and the susceptibility of the reactants to form side reactions. In some aspects, the polymerization step (a) takes place at a temperature in the range of about −40° C. to about 160° C.; for solution polymerization, at a temperature in the range of about 0° C. to about 65° C.; for bulk polymerization, at temperatures of approximately 150° C.

The time required for the polymerization step (a) also can vary widely, depending upon the type of polymerization being used and the molecular weight desired. In some embodiments, the polymerization step (a) takes place in about 30 minutes to about 24 hours.

While the polymerization step (a) can be in bulk, in solution, by interfacial polycondensation, or any other convenient method of polymerization. In some aspects, the polymerization step (a) is a solution polymerization reaction. Particularly when solution polymerization reaction is used, an acid acceptor is advantageously present during the polymerization step (a). A particularly suitable class of acid acceptor comprises tertiary amines, such as pyridine, trimethylamine, triethylamine, substituted anilines, and substituted aminopyridines. An illustrative acid acceptor is the substituted aminopyridine 4-dimethyl-aminopyridine (“DMAP”).

The purpose of the copolymerization of step (b) is to form a block copolymer comprising (i) the phosphorylated homopolymer chains produced as a result of polymerization step (a), and (ii) interconnecting polyester units. The result is a block copolymer having a microcrystalline structure particularly well-suited to use as a controlled release polymeric matrix.

The copolymerization step (b) of the invention usually takes place at a slightly higher temperature than the temperature of the polymerization step (a), but also can vary widely, depending upon the type of copolymerization reaction used, the presence of one or more catalysts, the molecular weight desired, the solubility desired, and the susceptibility of the reactants to undesirable side reaction. However, when the copolymerization step (b) is carried out as a solution polymerization reaction, it typically takes place at a temperature in the range of about −40° C. to about 100° C. Typical solvents include methylene chloride, chloroform, or any of a wide variety of inert organic solvents.

The time required for the copolymerization of step (b) can also vary widely, depending upon the molecular weight of the material desired and, in general, the need to use more or less rigorous conditions for the reaction to proceed to the desired degree of completion. Typically, however, the copolymerization step (b) takes place during a time of about 30 minutes to about 24 hours.

The terephthalate-poly(phosphite) polymer produced, whether a homopolymer or a block copolymer, is isolated from the reaction mixture by conventional techniques, such as by precipitating out, extraction with an immiscible solvent, evaporation, filtration, crystallization, and the like. Typically, however, the polymer of formula V is both isolated and purified by quenching a solution of the polymer with a non-solvent or a partial solvent, such as diethyl ether or petroleum ether.

In another embodiment, the terephthalate polyester includes a phosphoester linkage that is a phosphonate. Suitable terephthalate polyester-poly(phosphonate) copolymers are described, for example, in U.S. Pat. Nos. 6,485,737 and 6,153,212 (Mao et al., “Biodegradable Terephthalate Polyester-Poly(Phosphonate) Compositions, Articles and Methods of Using the Same”). According to this embodiment, the polymeric material comprises recurring monomeric units shown in Formula XII: 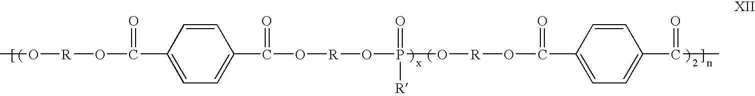
wherein R is a divalent organic moiety as defined above for terephthalate poly(phosphites) of formula V. R′ in the polymeric material of this embodiment is an aliphatic, aromatic, or heterocyclic residue. When R′ is aliphatic, it is preferably alkyl, such as methyl, ethyl, n-propyl, isopropyl, n-butyl, tert-butyl, —C₈H₁₇, and the like; or alkyl substituted with a non-interfering substituent, such as halogen, alkoxy, or nitro.

When R′ is aromatic, it typically contains about 5 to about 14 carbon atoms, or about 5 to about 12 carbon atoms and, optionally, can contain one or more rings that are fused to each other. Examples of suitable aromatic groups include phenyl, naphthyl, anthracenyl, phenanthranyl, and the like.

When R′ is heterocyclic, it typically contains about 5 to about 14 ring atoms, or about 5 to about 12 ring atoms, and one or more heteroatoms. Examples of suitable heterocyclic groups include furan, thiophene, pyrrole, isopyrrole, 3-isopyrrole, pyrazole, 2-isoimidazole, 1,2,3-triazole, 1,2,4-triazole, oxazole, thiazole, isothiazole, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-dioxazole, 1,2,4-dioxazole, 1,3,2-dioxazole, 1,3,4-dioxazole, 1,2,5-oxatriazole, 1,3-oxathiole, 1,2-pyran, 1,4-pyran, 1,2-pyrone, 1,4-pyrone, 1,2-dioxin, 1,3-dixoin, pyridine, N-alkylpyridinium, pyridazine, pyrimidine, pyrazine, 1,3,5-triazine, 1,2,4-triazine, 1,2,3-triazine, 1,2,4-oxazine, 1,3,2-oxazine, 1,3,5-oxazine, 1,4-oxazine, o-isoxazine, p-isoxazine, 1,2,5-oxathiazine, 1,2,6-oxathiazine, 1,4,2-oxadiazine, 1,3,5,2-oxadiazine, acepine, oxepin, thiepin, 1,2,4-diazepine, indene, isoindene, benzofuran, isobenzofuran, thionaphthene, isothinaphthene, indole, indolenin, 2-isobenzazole, 1,4-pyridein, pyrando-[3,4-b]-pyrrole, isoindazole, indoxazine, benzoxazole, anthranil, 1,2-benzopyran, 1,2-benzopyrone, 1,4-benzopyrone, 2,1-benzopyrone, 2,3-benzopyrone, quinoline, isoquinoline, 1,2-benzo-diazine, 1,3-benzodiazine, naphthyridine, pyrido-[3,4-b]pyridine, pyrido[3,2-b]-pyridine, pyrido-[4,3-b]pyridine, 1,3,2-benzoxazine, 1,4,2-benzoxazine, 2,3,1-benzoxazine, 3,1,4-benzoxazine, 1,2-benzisoxazine, 1,4-benzisoxazine, carbazole, xanthrene, acridine, purine, and the like. In some aspects, when R′ is heterocyclic, it is selected from the group consisting of furan, pyridine, N-alkyl-pyridine, 1,2,3- and 1,2,4-triazoles, indene, anthracene, and purine.

In one embodiment, R′ is an alkyl group or a phenyl group, or an alkyl group having 1 to 7 carbon atoms. In some particular embodiments, R′ is an ethyl group.

The value of x can be varied as described above for polymeric material containing phosphite ester linkages. Similarly, one method for controlling the value of x is to vary the feed ration of the “x” portion relative to the monomer. In this particular embodiment, feed ratios of the ethyl phosphonic dichloride “x” reactant (“EP”) can be used with the terephthaloyl chloride reactant (“TC”) to manufacture the polymer of formula XIII:

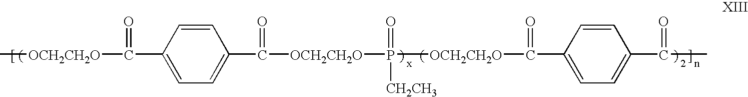

The most common general reaction in preparing a poly(phosphonate) is a dehydrochlorination between a phosphonic dichloride and a diol according to the following equation:

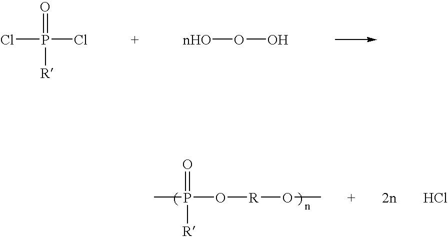

Bulk polycondensation, solution polycondensation, or interfacial polycondensation can be used to synthesize the polymers. A Friedel-Crafts reaction can also be used to synthesize poly(phosphonates). Polymerization typically is effected by reacting either bis(chloromethyl) compounds with aromatic hydrocarbons or chloromethylated diphenyl ether with triaryl phosphonates. Poly(phosphonates) can also be obtained by bulk condensation between phosphorus diimidazolides and aromatic diols, such as resorcinol and quinoline, usually under nitrogen or some other inert gas.

In one embodiment, the process of making the biodegradable terephthalate polymer of formula XIII comprises the steps of polymerizing p moles of a diol compound having formula VIII above, with q moles of a phosphonic dichloride of formula XIV: 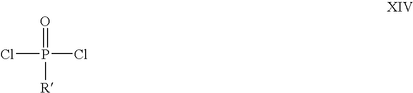
Wherein R′ is defined as above, and p>q, to form q moles of a homopolymer of formula XV shown below: 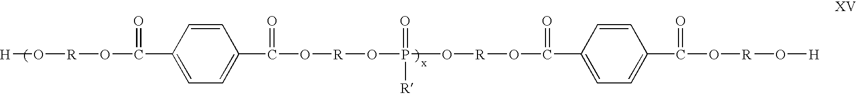
wherein R, R′ and x are as defined above. The homopolymer so formed can be isolated, purified and used as is. Alternatively, the homopolymer, isolated or not, can be used to prepare a block copolymer composition of the invention by: (a) polymerizing as described above, and (b) further reacting the homopolymer of formula XV and excess diol of formula VIII with (p-q) moles of terephthaloyl chloride having the formula XVI: 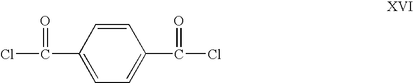
to form the copolymer of formula XII.

The function of the polymerization reaction of step (a) is to phosphorylate the di-ester starting material and then to polymerize it to form the homopolymer. As described above for polymeric material containing phosphite ester linkages, the polymerization step (a) can take place at widely varying temperatures and times.

The addition sequence of the polymerization step (a) can vary significantly depending upon the relative reactivities of the diol of formula VIII, the phosphonic dichloride of formula XIV, and the homopolymer of formula XV; the purity of these reactants; the temperature at which the polymerization reaction is performed; the degree of agitation used in the polymerization reaction; and the like. In some aspects, the diol of formula VIII is combined with a solvent and an acid acceptor, and the phosphonic dichloride is added slowly, for example, a solution of the phosphonic dichloride in a solvent can be trickled in or added dropwise to the chilled reaction mixture of diol, solvent, and acid acceptor, the control the rate of the polymerization reaction.

The purpose and conditions of the copolymerization of step (b) are as described above for polymeric material containing phosphite ester linkages.

The polymer of formula XII, whether a homopolymer or a block polymer, is isolated from the reaction mixture by conventional techniques, such as by precipitating out, extraction with an immiscible solvent, evaporation, filtration, crystallization, and the like. Typically, however, the polymer of formula XII is both isolated and purified by quenching a solution of the polymer with a non-solvent or a partial solvent, such as diethyl ether or petroleum ether.

The polymer of formula XII is usually characterized by a release rate of the bioactive agent in vivo that is controlled at least in part as a function of hydrolysis of the phosphoester bond or the polymer during biodegradation.

Further, the structure of the side chain can influence the release behavior of the polymer. For example, it is expected that conversion of the phosphorus side chain to a more lipophilic, more hydrophobic or bulky group would slow down the degradation process. Thus, for example, release is usually faster from copolymer compositions with a small aliphatic group side chain than with a bulky aromatic side chain.

The lifetime of a biodegradable polymer in vivo also depends upon its molecular weight, crystallinity, biostability, and the degree of crosslinking. In general, the greater the molecular weight, the higher the degree of crystallinity, and the greater the biostability, the slower biodegradation will be. Accordingly, degradation times can vary widely, for example, from less than one day to several months.

In another embodiment, the terephthalate polyester includes a phosphoester linkage that is a phosphate. Suitable terephthalate polyester-poly(phosphate) copolymers are described, for example, in U.S. Pat. Nos. 6,322,797 and 6,600,010 (Mao et al., “Biodegradable Terephthalate Polyester-Poly(Phosphate) Polymers, Compositions, Articles, and Methods for Making and Using the Same”). According to this embodiment, the polymeric material comprises recurring monomeric units shown in Formula XVII: 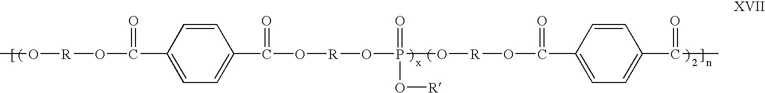
wherein R is a divalent organic moiety as described above for terephthalate poly(phosphites) of Formula V and terephthalate poly(phosphonates) of Formula XII. In some embodiments, R is an alkylene group, a cycloaliphatic group, a phenylene group, or a divalent group of the formula XVIII: 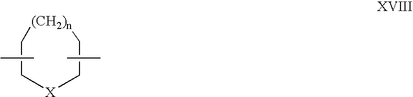
wherein X is oxygen, nitrogen, or sulfur, and n is 1 to 3. In some aspects, R is an alkylene group having 1 to 7 carbon atoms. In some embodiments, R is an ethylene group, a 2-methyl-propylene group, or a 2,2′ -dimethylpropylene group. R′ is as describe above for terephthalate poly(phosphites) of Formula V and terephthalate poly(phosphonates) of Formula XII, with the proviso that R′ could also comprise an alkyl conjugated to a biologically active substance to form a pendant bioactive agent delivery system. The value x is 1 or more and can vary as described for terephthalate poly(phosphites) of Formula V and terephthalate poly(phosphonates) of Formula XII. Similarly, one method for controlling the value of x is to vary the feed ratio of the “x” portion relative to the other monomer (for example, varying the feed ratios of the ethyl phosphorodichloridate “x” reactant (“EOP”) relative to the terephthaloyl chloride reactant (“TC”)). The value of n is 0 to 5,000 as described above terephthalate poly(phosphites) of Formula V and terephthalate poly(phosphonates) of Formula XII.

The most common general reaction in preparing poly(phosphates) is a dehydrochlorination between a phosphodichlorinate and a diol according to the following equation:

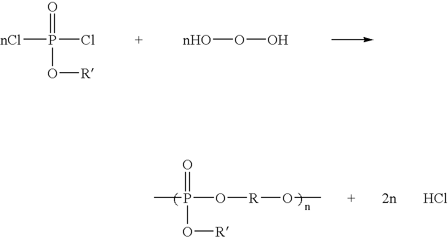

A Friedel-Crafts reaction can also be used to synthesize poly(phosphates). The principals described above for poly(phosphonates) can be utilized for synthesis of poly(phosphates) as well.

The polyphosphates can be synthesized via bulk polycondensation, solution polycondensation, and interfacial polycondensation as described above.

In one embodiment, the process of making a biodegradable terephthalate homopolymer of formula XVII comprises the step of polymerizing p moles of a diol compound having formula XIX: 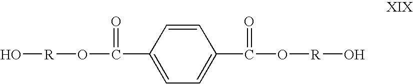
wherein R is as defined above, with q moles of a phosphorodichloridate of formula XX: 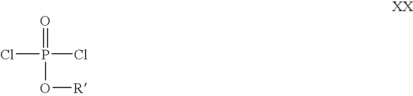
wherein R′ is defined above, and p>q, to form q moles of a homopolymer of formula XXI as shown below: 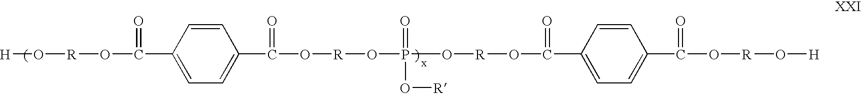
wherein R, R′ and x are as defined above. The homopolymer so formed can be isolated, purified and used as is. Alternatively, the homopolymer, isolated or not, can be used to prepare a block copolymer by (a) polymerizing as described above, and (b) further reacting the homopolymer of formula XXI and excess diol of formula XIX with (p-q) moles of terephthaloyl chloride having the formula XVI to form the polymer of formula XVII.

The function of polymerization steps (a) and (b), as well as conditions therefor are as described above for poly(phosphonates). The addition sequence for the copolymerization step (b) can vary significantly depending upon the relative reactivities of the homopolymer of Formula XXI and the terephthaloyl chloride of Formula XVI; the purity of these reactants; the temperature at which the copolymerization reaction is performed; the degree of agitation used in the copolymerization reaction; and the like. In some aspects, the terephthaloyl chloride of Formula XVI is added slowly to the reaction mixture, rather than vice versa. For example, a solution of the terephthaloyl chloride in a solvent can be trickled in or added dropwise to the chilled or room temperature reaction, to control the rate of the copolymerization reaction.

The polymeric materials comprising a biodegradable terephthalate copolymer that includes a phosphorus-containing linkage (poly(phosphates), poly(phosphonates) and poly(phosphites)) can comprise additional biocompatible monomeric units so long as they do not interfere with the biodegradable characteristics of the polymeric material. Such additional monomeric units can, in some embodiments, offer even greater flexibility in designing the precise release profile desired for targeted bioactive agent delivery or the precise rate of biodegradability desired for structural implants. Examples of such additional biocompatible monomers include, but are not limited to, the recurring units found in polycarbonates, polyorthoesters, polyamides, degradable polyurethanes, poly(iminocarbonates), and polyanhydrides.

In some aspects of the invention, the polymeric material of these embodiments is soluble in one or more common organic solvents for ease of fabrication and processing. Common organic solvents can include chloroform, dichloromethane, acetone, ethyl acetate, dimethyl acetamide (DMAC), N-methyl pyrrolidone, dimethylformamide, and dimethylsulfoxide. In particular embodiments, the polymeric material is soluble in at least one of these solvents.

The Tg of the polymeric material according to these embodiments can vary widely depending upon the branching of the diols used to prepare the polymer, the relative proportion of phosphorus-containing monomer used to make the polymer, and the like. However, in some aspects, the Tg is within the range of about −10° C. to about 100° C., or in the range of about 0° C. to about 50° C.

When working with poly(phosphates) and poly(phosphonates), the structure of the side chain can influence the release behavior of the polymer. For example, it is generally expected that, with the classes of poly(phosphoesters) described herein, conversion of the phosphorus side chain to a more lipophilic, more hydrophobic or bulky group would slow down the degradation process. For example, release would usually be faster from copolymer compositions with a small aliphatic group side chain than with a bulky aromatic side chain.

The terephthalate poly(phosphites) of formula V are usually characterized by a release rate of the bioactive agent in vivo that is controlled at least in part as a function of hydrolysis of the phosphoester bond of the polymer during biodegradation. However, poly(phosphites) do not have a side chain that can be manipulated to influence the rate of biodegradation.

In the case of biodegradable terephthalate poly(phosphite) polymer in vivo depends sufficiently upon its molecular weight, crystallinity, biostability, and the degree of cross-linking to achieve acceptable degradation rates. In general, the greater the molecular weight, the higher the degree of crystallinity, and the greater the biostability, the slower biodegradation will be.

In still further embodiments of the invention, the polymeric material comprises a copolymer comprising a biodegradable, segmented molecular architecture that includes at least two different ester linkages. According to these particular embodiments, the polymeric material can comprise block copolymers (of the AB or ABA type) or segmented (also known as multiblock or random-block) copolymers of the (AB)ₙ type. These copolymers are formed in a two (or more) stage ring opening copolymerization using two (or more) cyclic ester monomers that form linkages in the copolymer with greatly different susceptibilities to transesterification. These embodiments are described, for example, in U.S. Pat. No. 5,252,701 (Jarrett et al., “Segmented Absorbable Copolymer”) and will now be described in some detail herein.

In one aspect, the polymeric material comprises a copolymer comprising a biodegradable, segmented molecular architecture that includes at least two different ester linkages. Generally speaking, the segmented molecular architecture comprises a plurality of fast transesterifying linkages and a plurality of slow transesterifying linkages. The fast transesterifying linkages have a segment length distribution of greater than 1.3. Sequential addition copolymerization of cyclic ester monomers is utilized in conjunction with a selective transesterification phenomenon to create biodegradable copolymer molecules with specific architectures.

The sequential addition polymerization process of this embodiment is a two (or more) stage ring opening copolymerization using two (or more) cyclic ester monomers that form linkages in the copolymer with greatly different susceptibilities towards transesterification (also referred to herein as “selective transesterification”). For example, such a pair of monomers is ε-caprolactones which forms slow reacting (transesterifying) caproate linkages and glycolide that forms fast reacting glycolate linkages when conventional tin catalysts are employed.

Other parent monomers that can be useful in this process include: p-dioxanone, dioxepanone, deltavalerolactone, beta-butyrolactone, ε-decalactone, 2,5-diketomorpholine, pivalolactone, alpha, alpha-diethylpropiolactone, 6,8-dioxabicyclo octane-7-one, ethylene carbonate, ethylene oxalate, 3-methyl-1,4-dioxane-2,5-dione, 3,3-dimethyl 1,4-dioxane-2,5-dione, substituted glycolides, and substituted lactides. Other cyclic esters described in the art can also be employed with the scope of this invention. These monomers can be categorized as to their susceptibility towards transesterification.

The first stage (Stage I) of the copolymerization consists of a statistical copolymer that has a high content of the slower transesterifying (for example, caproate) linkages and a low content of fast reaction (for example, glycolate) linkages. This prepolymer forms a framework of segments consisting of runs of consecutive caproate linkages with interspersed short glycolate segments. The length and distribution of these segments is affected by such factors as monomer feed composition, the reactivity ratios of the monomers, and the degree of transesterification that occurs in this stage of the reaction. This framework, then, consists of segments with different reactivities for transesterification.

The second stage (Stage II) of the copolymerization consists of the addition of the faster reacting monomer (for example, glycolide) and continuation of the reaction for a specified length of time. The difference in transesterification reactivities of the two segments in the prepolymer preserves the caproate segments in the final copolymer. The second stage initially forms long glycolate segments, most likely at the ends of the Stage I prepolymer. Through transesterification, glycolate linkages from the initially long Stage II glycolate segments are gradually transferred into the shorter glycolate segments in the Stage I prepolymer. The result is a more narrow distribution of glycolate segment lengths. The resulting copolymer has a segmented architecture, which is determined by the Stage I prepolymer framework, the final composition and the difference in transesterification rates. The distribution of segment lengths changes as a function of time after addition of the second stage. This distribution has a marked effect on material properties. In this way, a wide range of material properties can be easily achieved by varying the reaction time for the second and subsequent stages.

This mechanism is not necessarily limited to the caprolactone-glycolide pair. It is known that trimethylene carbonate shows similar behavior to caprolactone when copolymerized with glycolide, and 1-lactide behaves similarly to glycolide when copolymerized with trimethylene carbonate. The observed differences in transesterification rates can be due to the interaction of the linkages with the catalyst. Without intending to be bound by a particular theory, it is believed that linkages within the polymer chain that promote coordination with the catalyst complex would be expected to be more susceptible to undergo transesterification reactions. Such linkages are termed “fast reacting” linkages. It is believed that any combination of a linkage having a fast transesterification rate with a linkage having a slow transesterification rate (or “slow reacting linkage”) can be used to prepare specific architectures in a copolymer of those linkages.

Given the above reasoning, monomers, and the linkages formed from them, can be categorized according to their predicted susceptibilities toward transesterification. The following monomers would be expected to form fast reacting linkages: glycolide, lactide (1, d, dl, or meso), 3-methyl-1,4-dioxane-2,5-dione, 3,3-diethyl-1,4-dioxan-2,5-dione, combinations of any of these, and other substituted “glycolide” type monomers. Thus, in some embodiments, the fast transesterifying linkages are selected from lactate linkages, glycolate linkages, lactate and glycolate linkages.

The following monomers would be expected to form slow reacting linkages: 1,4-dioxan-2-one (hereafter referred to as “dioxanone linkages”), 1,4-dioxepan-2-one, 1,5-dioxepan-2-one, delta-valerolactone ε-decalactone, pivalolactone, gamma-butyrolactone, ethylene carbonate, trimethylene carbonate, ε-caprolactone, 6,8-dioxabicyclooctane-7-one. Other monomers known to copolymerize should be categorizable according to their reactivities. The reactivities of some of these monomers, however, are difficult to predict. These monomers include: 2,5-diketomorpholine, beta-butyrolactone, propiolactone, and ethylene oxalate. Other cyclic esters described in the art can also be employed with the scope of this invention. The above categorizations are based upon theory, and actual categorization of reactivities can be accomplished experimentally. In some embodiments, the slow transesterifying linkages are selected from trimethylene carbonate, caproate, and dioxanone linkages.

Determination of whether a monomer comprises a fast or slow transesterifying linkage can involve the following test. A copolymer of the monomer of interest and glycolide are prepared using the sequential addition method. The copolymer is made with 100% monomer in the first stage and 100% glycolide (GLY) in the second stage. The following reaction conditions are employed:

Stage I

| | |
|---|---|
| Time | 40 minutes |
| Temperature | 165° C. for 25 minutes, then |
| | increased to 180° C. over 15 minutes |
| Charge | Monomer: 65.10 g |
| | SnCl₂2H₂O: 4.09 mg |
| | Diethylene glycol: 7.8 μl |

Stage II

| | |
|---|---|
| Time | 2 hours |
| Temperature | 180° C. to 210° C. over 30 minutes |
| | 210° C. for 1.5 hours |
| Charge | Gly 134.9 g |

The resulting copolymer is ground and placed in vacuum oven at 110° C., <1 mmHg overnight. Thermal analysis and ¹³C NMR analysis are then performed on the sample. If the block length is equal to or greater than 30, the final glycolate weight percent is 68%, and the inherent viscosity is about 1.0 dL/g, then the monomer comprises a slow transesterifying linkage. An inherent viscosity substantially less than about 1.0 dL/g, means that the polymer formed is unstable at the test conditions.

In some aspects, the copolymer has an inherent viscosity of greater than about 0.1 dL/g (concentration of 0.5 g/dL in a solvent, for example hexafluoroacetone sesquihydrate). For an article of manufacture, such as a surgical suture, requiring an industry acceptable tensile (or other) strength value, an inherent viscosity of about 1.0 dL/g (0.5 dL/g in a solvent) or greater can be utilized. For an article of manufacture such as a controlled release device, where a strength value is not required, the copolymer can have an inherent viscosity of lower than about 1.0 dL/g (0.5 g/dL in a solvent).

In some embodiments, the copolymer includes lactate linkages having a crystallinity of less than about 40% based upon differential scanning calorimetry and a melting point of less than about 170° C. Still another embodiment of the copolymer includes glycolate linkages having a crystallinity of less than about 30% based upon differential scanning calorimetry and a melting point of less than about 215° C. In a more specific embodiment, the copolymer comprises a bioabsorbable, segmented molecular architecture having a plurality of lactate linkages. The segment length distribution of the lactate linkages is greater than 1.3, the crystallinity is less than about 40% based upon differential scanning calorimetry and the melting point of the copolymer is less than about 170° C. The segmented molecular architecture also has a plurality of trimethylene carbonate linkages.

According to the invention, the copolymer can be manufactured by sequential addition of at least two different cyclic ester monomers in at least two stages. The first cyclic ester monomer is selected from carbonates and lactones, and mixtures thereof. The second cyclic ester monomer is selected from lactides and mixtures thereof. The sequential addition comprises the following steps:
(1) first polymerizing a first stage at least the first cyclic ester monomer in the presence of a catalyst at a temperature in the range of about 160° C. to about 220° C. to obtain a first polymer melt;
(2) adding at least the second cyclic ester monomer to the first polymer melt; and
(3) copolymerizing in a second stage the first polymer melt with at least the second cyclic ester monomer to obtain a second copolymer melt.

The process also comprises transesterifying the second copolymer melt for up to about 5 hours at a temperature of greater than about 180° C.

In one embodiment of the process, the first polymerization step comprises polymerizing in the first stage from about 80 mole % of the first cyclic ester monomer. The remaining mole %, if any, comprises the second cyclic ester monomer. In another embodiment of the process, the first polymerizing step comprises polymerizing in the first stage up to about 90 mole % of the first cyclic ester monomer. In still another embodiment of the process, the step of adding at least the second cyclic ester monomer to the first polymer melt comprises adding more than about 80 mole % of the second cyclic ester monomer. The remaining mole percentage, if any, comprises the first cyclic ester monomer. In a specific embodiment of the process, the step of adding at least the second cyclic ester monomer to the first polymer melt comprises adding 100 mole % of the second cyclic ester monomer.

Another process for manufacturing a copolymer having a biodegradable, segmented molecular architecture comprises sequential addition of at least two different cyclic ester monomers in three stages. The first cyclic ester monomer is selected from carbonates, lactones, and mixtures of carbonates and lactones. The second cyclic ester monomer is selected from lactides and mixtures thereof. The sequential addition comprises the following steps:
(1) first polymerizing in a first stage at least the first cyclic ester monomer in the presence of a catalyst at a temperature in the range of about 160° C. to about 220° C. to obtain a first polymer melt;
(2) first adding at least the second cyclic ester monomer to the first polymer melt;
(3) second copolymerizing in a second stage the first polymer melt with at least the second cyclic ester monomer to obtain a second copolymer melt;
(4) second adding at least the second cyclic ester monomer to the second copolymer melt; and
(5) copolymerizing in a third stage the second copolymer melt with at least the second cyclic ester monomer to obtain a third copolymer melt.

The process also comprises transesterifying the third copolymer melt from up to about 5 hours at a temperature of greater than about 180° C.

In one embodiment of this three-stage process, the first polymerizing step comprises polymerizing in the first stage about 80 mole % or more of the first cyclic ester monomer. The remaining mole percentage, if any, comprises the second cyclic ester monomer. In another embodiment, the first stage comprises polymerizing up to about 90 mole % of the first cyclic ester monomer. In still another embodiment, the addition of the second cyclic ester monomer to the first polymer melt and/or the addition of the second cyclic ester monomer to the second copolymer melt comprise adding more than about 80 mole % of the second cyclic ester monomer. The remaining mole percentage, if any, comprises the first cyclic ester monomer. In a specific embodiment of the process, the addition of the second cyclic ester monomer to the first polymer melt and/or the addition of the second cyclic ester monomer to the second copolymer melt comprises adding 100 mole % of the second cyclic ester monomer.

Optionally, the process can involve polymerization in the presence of a metal coordination catalyst and/or an initiator. In some embodiments, the initiator can be selected from monofunctional and polyflnctional alcohols.

It is understood the catalyst type and level of catalyst employed will affect both the relative polymerization and transesterification rates of the cyclic esters of the invention. By proper choice of both catalyst type and level, copolymers with specific architecture can be prepared in a controllable manner and within a reasonable amount of time. Catalysts such as stannous octoate or stannous chloride dihydrate can be utilized. Other catalysts known in the art to be effective in the ring opening polymerization of cyclic esters are also suitable in accordance with these embodiments of the invention.

The types of architectures that can be made utilizing this process can be AB diblock, ABA triblock, or segmented copolymers with wide or narrow block length distributions. Diblocks and triblocks are made using monofunctional or diflnctional initiators (alcohols) in the Stage I reaction and by using only the slow transesterification rate linkage to form a Stage I homopolymer. The Stage II linkages can only transesterify within the Stage II segment, preserving the diblock or triblock architecture.

It is generally preferred to conduct the sequential polymerization in a single reaction vessel, by sequentially adding the monomers thereto. However, if desired, one or more of the stages can be polymerized in separate reaction vessels, finally combining the stages for transesterification in a single reaction vessel. Such a process would allow the use of a cyclic polyester forming monomers for one or more of the stages.

Transesterification in aliphatic polyesters derived from cyclic monomers is known in the art. For example, Gnanou and Rempp, Macromol. Chem., 188:2267-2275 (1987) have described the anion polymerization of e-caprolactone in the presence of lithium alkoxides as being a living polymerization that is accompanied by simultaneous reshuffling. According to this reference, if reshuffling occurs between two different molecules, it can be referred to as “scrambling.” If reshuffling occurs intramolecularly, it is called “back-biting,” and it results in the formation of cycles, the remaining linear macromolecules are of lower molecular weight, but they still carry an active site at the chain end.

According to these embodiments of the invention, copolymers containing certain ester linkages are susceptible to varying degrees to transesterification (or reshuffling) reactions. When linkages of greatly different susceptibilities are present (such as caproate and glycolate), reshuffling or transesterification reactions occur primarily with the faster reacting (glycolate) linkages. Similar to the number average molecular weight of the homopolymer described by Gnnaou and Rempp, in this instance reshuffling leads to little or no change in the number average segment lengths, as long as the composition is unchanged by these or other reactions. Similar to the molecular weight distribution effect described by Gnnaou and Rempp, in this instance reshuffling tends to change the segment length distribution, in the direction of a Schultz-Flory or most probable distribution.

Thus a prepolymer (or Stage I polymer) can serve as a framework (or template) containing linkages with widely different susceptibility towards transesterification. The Stage I polymer contains predominantly slow reacting linkages. Addition of a second stage (a second monomer addition) consisting of predominantly fast reacting linkage forming monomer results in polymerization of the Stage II monomer initiated by the Stage I/catalyst complex, and transesterification (reshuffling) consisting predominantly of fast reacting linkage reactions leading to a narrowing of the fast reacting linkage segment length distribution over time.

After full conversion of the Stage II monomer to polymer, the number average segment lengths show little or no change as a consequence of the reshuffling reactions. As the reaction proceeds the architecture of the copolymer is determined by several reaction variables. For example, the concentration of the fast reacting linkages in the Stage I copolymer can impact the architecture of the copolymer. As the concentration of fast reacting linkages in the Stage I copolymer is increased, the transesterification reaction rate during the second, and subsequent, stages increases. Also, the catalyst type and concentration can impact the architecture of the copolymer. The particle catalyst and level of catalyst employed determines the relative reactivities of the ester linkages, and the transesterification rate. Further reaction temperature and time can impact copolymer architecture. Reaction temperature and time will determine the rate and extent of the transesterification reactions and resulting segment length distribution.

The average segment lengths can be determined utilizing concepts included in Kricheldorf et al. (Macromolecules, 2173-2181(1984) and U.S. Pat. No. 5,252,701.

The copolymer having a segmented molecular architecture as described in the above embodiments can be utilized to fabricate an implantable medical device. The implantable medical device can be fabricated using such standard techniques, including extrusion techniques. In some embodiments, extrusion pellets or resin comprising the copolymer can be used in dry spinning and wet spinning (including gel spinning). Examples of products that can be manufactured from the extrusion pellets or resin include, but are not limited to, a fiber, film, and/or tubing including a porous hollow tube. In some embodiments, the implantable device comprises at least one filament. In another embodiment, the implantable device comprises a controlled release device. In a specific embodiment, the controlled release device comprises a plurality of microsphere. The microspheres can be dispersed in a pharmaceutically and pharmacologically acceptable liquid to obtain a slow release composition.

In still further embodiments, the biodegradable polymeric matrix comprises a random copolymer comprising at least one carbonate unit as the major component, the carbonate copolymerized with at least one second monomeric component. According to these embodiments, certain aliphatic carbonates can form highly crystalline random copolymers with other monomer components, so long as the appropriate carbonate is present as the major component. These copolymers can provide one or more advantages, such as relatively high modulus and tensile strength, controllable biodegradation rates, blood compatibility, and biocompatibility with living tissue. In some aspects, these copolymers also induce minimal inflammatory tissue reaction, as biodegradation of the carbonate polymer by hydrolytic depolymerization results in degradation substances having physiologically neutral pH. Exemplary random copolymers are described, for example, in U.S. Pat. No. 4,891,263 (Kotliar et al.), U.S. Pat. No. 5,120,802 (Mares et al.), U.S. Pat. No. 4,916,193 (Tang et al.), U.S. Pat No. 5,066,772 (Tang et al.), and U.S. Pat. No. 5,185,408 (Tang et al.).

According to these embodiments, the copolymers are random copolymers comprising as a minor component one or more recurring monomeric units, and as a major component, a recurring carbonate monomeric unit of the following general structures (XXII): 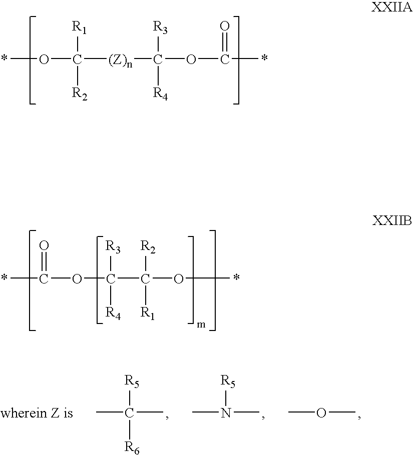
or combinations thereof, where Z is selected such that there are no adjacent heteroatoms;

n and m are the same or different and are integers from about 1 to about 8; and

R₁, R₂, R₃, and R₄ are the same or different at each occurrence and are hydrogen, alkoxyaryl, aryloxyaryl, arylalkyl, alkyarylalkyl, arylalkylaryl, alkylaryl, arylcarbonylalkyl, aryloxyalkyl, alkyl, aryl, alkylcarbonylalkyl, cycloalkyl, arylcarbonylaryl, alkylcarbonylaryl, alkoxyalkyl, or aryl or alkyl substituted with one or more biologically compatible substituents such as alkyl, aryl, alkoxy, aryloxy, dialkyamino, diarylamino, alkylarylamino substituents;

R₅ and R₆ are the same or different and are R₁, R₂, R₃, R₄, dialkylamino, diarylamino, alkylarylamino, alkoxy, aryloxy, alkanoyl, or arylcarbonyl; or any two of R₁ to R₆ together can form an alkylene chain completing a 3, 4, 5, 6, 7, 8, or 9 membered monocyclic, alicyclic, spiro, bicyclic, and/or tricyclic ring system, which system can optionally include one or more non-adjacent carbonyl, oxa, alkylaza, or arylaza groups;

with the proviso that at least one of R₁ to R₆ is other than hydrogen.

Illustrative of useful R₁, R₂, R₃, and R₄ groups are hydrogen; alkyl such as methyl, ethyl, propyl, butyl, pentyl, hexyl, septyl, octyl, nonyl, tert-butyl, neopentyl, isopropyl, sec-butyl, dodecyl, and the like; cycloalkyl such as cyclohexyl, cyclopentyl, cyclooctyl, cycloheptyl, and the like; alkoxyalkyl such as methoxymethylene, ethoxyrnethylene, butoxymethylene, propoxyethylene, pentoxybutylene, and the like; aryloxyalkyl and aryloxyaryl such as phenoxyphenylene, phenoxymethylene and the like; and various substituted alkyl and aryl groups such as 4-dimethylaminobutyl, and the like.

Illustrative of other R₁ to R₄ groups are divalent aliphatic chains, which can optionally include one or more oxygen, trisubstituted amino or carbonyl groups, such as —(CH₂)₂—, —CH₂(O)CH₂—, —(CH₂)₃—, —CH₂—CH(CH₃)—, —(CH₂)₄—, —(CH₂)₅—, —CH₂OCH₂—, —(CH₂)₂—N(CH₃)CH₂—, —CH₂C(O)CH₂—, —(CH₂)₂—N(CH₃)—CH₂)₂—, and the like, and divalent chains to form fused, spiro, bicyclic or tricyclic ring systems, such as —CH(CH₂CH₂)₂CH—, —CH(CH₂CH₂CH₂)₂CH—, —CH(CH₂)(CH₂CH₂)CH—, —CH(CH₂)(CH₂—CH₂CH₂)CH—, —CH(C(CH₃)₂)(CH₂CH₂)CH—, and the like.

Illustrative of useful R₅ and R₆ groups are the above-listed representative R₁ to R₄ groups, including OCH₂C(O)CH₂, (CH₂)₂—NCH₃—, —OCH₂C(O)CH₂—, —O—(CH₂)₂—O—, alkoxy such as propoxy, butoxy, methoxy, isopropoxy, pentoxy, nonyloxy, ethoxy, octyloxy, and the like; dialkylamino such as dimethylamino, methylethylamino, diethylamino, dibutylamino, and the like; alkanoyl such as propanoyl, acetyl, hexanoyl, and the like; arylcarbonyl such as phenycarbonyl, p-methylphenyl carbonyl, and the like; and diarylamino and arylalkylamino such as diphenylamino, methylphenylamino, ethylphenylamino, and the like.

Illustrative copolymers in accordance with these embodiments are random copolymers comprising as a major component, carbonate recurring units of the structure illustrated in Formula XXIIA, wherein Z is —(R₅—C—R₆)—, or a combination thereof; n is 1, 2, or 3; and R₁ to R₆ are as defined above, preferably where aliphatic moieties included in R₁ to R₆ include up to about 10 carbon atoms and the aryl moieties include up to about 16 carbon atoms.

Illustrative of these copolymers are those wherein, in the major component, n is 1 and Z is of the formula XXIII: 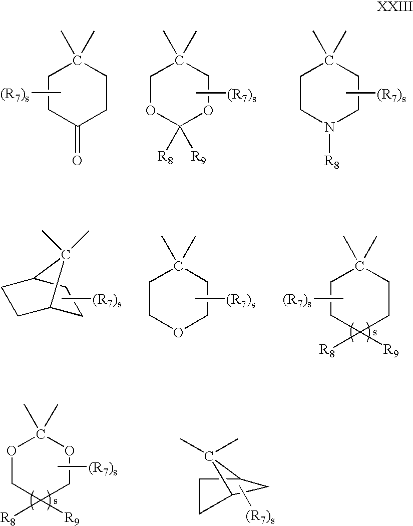
where —C— denotes the center carbon atom of Z, when Z is —C(R₅)(R₆)—; R₇ is the same or different and is aryl, alkyl or an alkylene chain completing a 3 to 16 membered ring structure, including fused, spiro, bicyclic and/or tricyclic structures, and the like; R₈ and R₉ are the same or different at each occurrence and are R₇ or hydrogen, and s is the same or different at each occurrence and is 0 to 3, and the open valencies are substituted with hydrogen atoms.

Also illustrative of these major components are those comprising recurring units of the formula XXIV: 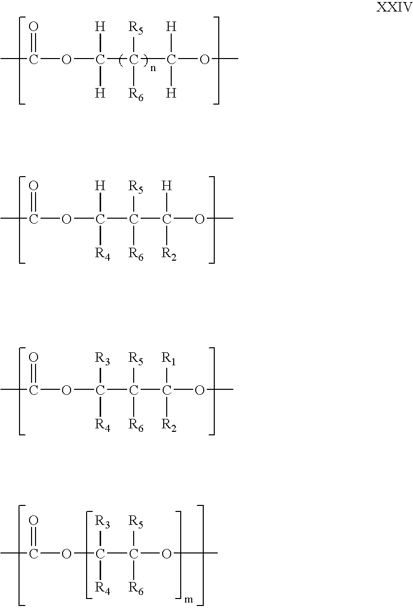
wherein:

R₁, R₂, R₃, and R₄, are the same or different at each occurrence and are hydrogen, alkyl such as methyl, ethyl, n-propyl, isopropyl, n-butyl, sec-butyl, t-butyl, neopentyl, and the like; phenyl; anisyl; phenylalkyl, such as benzyl, phenethyl, and the like; phenyl substituted with one or more alkyl or alkoxy groups such as tolyl, xylyl, p-methoxyphenyl, m-ethoxyphenyl, p-propoxyphenyl, and the like; and alkoxyalkyl such as methoxymethyl, ethoxymethyl, and the like; R₅ and R₆ are the same or different and are R₁ to R₄; alkoxy, alkanoyl, arylcarbonyl, dialkylamino; or any two of R₁ to R₆ together can form alkylene chain completing 4, 5, 6, 7, 8, or 9 membered monocyclic, spiro, bicyclic and/or tricyclic ring structure which structure can optionally include one or more non-adjacent divalent carbonyl, oxa, alkylaza, or arylaza groups with the proviso that at least one of R₁ or R₆ is other than hydrogen; and

n and m are the same or different and are 1, 2, or 3.

Illustrative copolymers for use in these embodiments are random copolymers comprising as a major component, recurring units of the formula XXV: 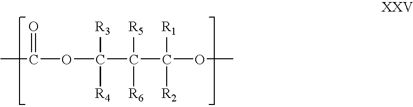
wherein:

R₁ to R₄ are the same or different and are alkyl, hydrogen, alkoxyalkyl, phenylalkyl, alkoxyphenyl, or alkylphenyl, wherein the aliphatic moieties include 1 to 9 carbon atoms; and

R₅ and R₆ are the same or different at each occurrence and are selected from the group of R₁ to R₄ substituents, aryloxy, and alkoxy, or R₅ and R₆ together can form an aliphatic chain completing a 3 to 1 membered spiro, bicyclic, and/or tricyclic structure which can include one or two non-adjacent oxa, alkylaza, or arylaza groups, with the proviso that at least one of R₁ to R₄ is other than hydrogen.

Preferably, the random copolymer comprises as a major component, recurring monomeric units of the following formula XXVI: 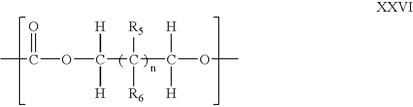
wherein:

n is 1;
R₅ and R₆ are the same or different and are hydrogen, phenyl, phenylalkyl, or phenyl or phenylalkyl substituted with one or more alkyl or alkoxy groups; or alkyl or R₅ and R₆ together make a divalent chain forming a 3 to 6 membered spiro, bicyclic, and/or tricyclic ring structure which can include one or two non-adjacent carbonyl, oxa, alkylaza, or arylaza groups, with the proviso that at least one of R₅ and R₆ is other than hydrogen.

In some aspects of the invention, the random copolymer comprises as a major component, recurring monomeric units of Formula XXVI, particularly when R₅ and R₆ are the same or different and are alkyl, phenyl, phenylalkyl, or phenyl or phenylalkyl substituted with one or more alkyl or alkoxy groups; or a divalent chain forming a 3 to 10 membered, preferably 5 to 7 membered, spiro or bicyclic ring structure that can optionally include one or two non-adjacent oxa, carbonyl, or disubstituted amino groups. It can be preferred that R₅ and R₆ are the same or different and are phenyl, alkylphenyl or phenylalkyl such as tolyl, phenethyl or phenyl, or lower alkyl of 1 to 7 carbon atoms such as methyl, ethyl, propyl, isopropyl, n-butyl, tertiary butyl, pentyl, neopentyl, hexyl, and secondary butyl.

In particular embodiments utilizing Formula XXVI, R₅ and R₆ are the same or different, and are lower alkyl having about 1 to about 4 carbon atoms, and do not differ from each other by more than about 3 carbon atoms, and preferably by not more than about 2 carbon atoms. In some aspects, R₅ and R₆ are the same and comprise alkyl of about 1 to 2 carbon atoms, and in some aspects, methyl for each of R₅ and R₆.

According to these embodiments, the copolymers include a minor component comprising one or more other recurring monomer units. The minor component of the random copolymers of the invention can vary widely.

Illustrative of the second recurring monomeric components are those derived from carbonates, including but not limited to certain of the monomeric units included within the scope of Formula XXIIA wherein n is 0 to 8 within (Z)ₙ, and Formula XXIIB and Formula XXVI, wherein n=1, particularly those less preferred as the major component, and those derived from substituted or nonsubstituted ethylene carbonates, tetramethylene carbonates, trimethylene carbonates, pentamethylene carbonates, and the like. Also illustrative of the second recurring monomeric unit are those that are derived from monomers that polymerize by ring opening polymerization as, for example, substituted and unsubstituted beta, gamma, delta, omega, and other lactones such as those of the formula XXVII: 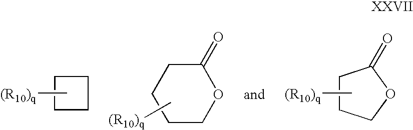
where R₁₀ is alkoxy, alkyl or aryl, and q is 0 to 3, wherein the open valencies are substituted with hydrogen atoms. Such lactones include caprolactones, valerolactones, butyrolactones, propiolactones, and the lactones of hydroxy carboxylic acids such as 3-hydroxy-2-phenylpropanoic acid, 3-hydroxy-3-phenylpropanoic acid, 3-hydroxybutanoic acid, 3-hydroxy-3-methylbutanoic acid, 3-hydroxypentanoic acid, 5-hydroxypentanoic acid, 3-hydroxy-4-methylheptanoic acid, 4-hydroxyocatnoic acid, and the like; and lactides such as l-lactide, d-lactide, d,l-lactide; glycolide; and dilactones such as those of the formula XXVIII: 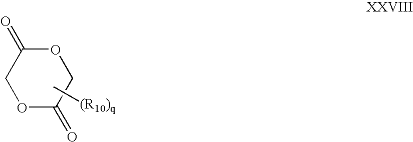
where R₁₀ and q are as defined above in Formula XXVII, and where the open valencies are substituted with hydrogen atoms. Such dilactones include the dilactones of 2-hydroxybutyric acid, 2-hydroxy-2-phenylpropanoic acid, 2-hydroxyl-3-methylbutanoic acid, 2-hydroxypentanoci acid, 2-hydroxy-4-methylpentanoic acid, 2-hydroxyhexanoic acid, 2-hydroxyoctanoic acid, and the like.

Illustrative of still further useful minor components are units derived from dioxepanones such as those described in U.S. Pat. No. 4,052,988 and U.K. Patent No. 1,273,733. Such dioxepanones include alkyl and aryl substituted and unsubstituted dioxepanones of the formula XXIX: 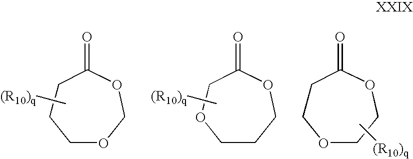
and monomeric units derived from dioxanones such as those described in U.S. Pat. Nos. 3,952,016, 4,052,988, 4,070,375, and 3,959,185, as for example, alkyl or aryl substituted and unsubstituted dioxanones of the formula XXX: 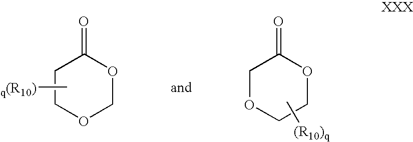
wherein q is as defined above; R₁₀ is the same or different at each occurrence and are hydroxycarbonyl groups such as alkyl and substituted alkyl, and aryl or substituted aryl; and the open valencies are substituted with hydrogen atoms. Preferably R₁₀ is the same or different and are alkyl groups containing 1 to 6 carbon atoms, preferably 1 or 2 carbon atoms, and q is 0 or 1.

Suitable minor components also include monomeric units derived from ethers such as 2,4-dimethyl-1,3-dioxane, 1,3-dioxane, 1-,4-dioxane, 2-methyl-5-methoxy-1,3-dioxane, 4-methyl-1,3-dioxane, 4-methyl-4-phenyl-1,3-dioxane, oxetane, tetrahydrofuran, tetrahydropyran, hexamethylene oxide, heptamethylene oxide, octamethylene oxide, nonamethylene oxide, and the like.

Still further minor components include monomeric units derived from epoxides such as ethylene oxide, propylene oxide, alkyl substituted ethylene oxides such as ethyl, propyl, and butyl substituted ethylene oxide, the oxides of various internal olefins such as the oxides of 2-butene, 2-pentene, 2-hexene, 3-hexene, an like epoxides; and also including units derived from epoxides with carbon dioxide; and monomeric units derived from orthoesters or orthocarbonates such as alkyl or aryl substituted or unsubstituted orthoesters, orthocarbonates, and cyclic anhydrides which may optionally include one or more oxa, alkylaza, arylaza, and carbonyl groups of the formula XXXI: 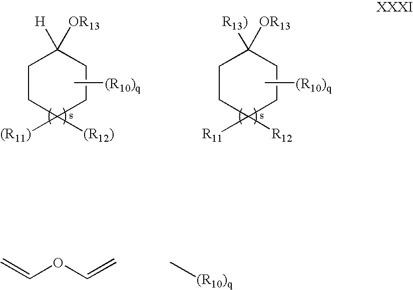
where q and R₁₀ are as described above, r is 0 to about 10, R₁₃ is the same or different at each occurrence and is alkyl or aryl, and R₁₁ and R₁₂ are the same or different and are hydrogen, alkyl or aryl.

Monomeric units derived from precursors and derivatives of lactides, lactones, dioxanones, orthoesters, orthocarbonates, anhydrides, and dioxepanones such as the various hydroxycarboxylic acids, substituted or non-substituted diacids such as oxa, aza, alkyl, aryl, hydroxy substituted oxacarboxylic acid acids, functionalized esters, and acid halide derivatives, and the like can also be used as the minor component.

Relative percentages of each of the recurring monomeric units that make up the copolymers of these embodiments can vary widely. The only requirement is that at least one type of recurring monomeric unit within the scope of Formula XXIIA be in the major amount, and that the other type of recurring unit or units be in the minor amount. As used herein, “major amount” is more than about 50 weight % based upon the total weight of all recurring monomeric units in the copolymer and “minor amount” is less than about 20 weight % based upon the total weight of all recurring monomeric units in the copolymer.

In addition, for certain applications, end-capping of these biopolymers can be desirable. End-capping can be accomplished by, for example, acylating, alkylating, silylating agents and the like.

In some embodiments, the random copolymers of these embodiments can be spun into fibers by any suitable fiber-forming technique, which fibers can then be fabricated in medical devices using conventional techniques. For example, once the random copolymers are formulated, the copolymers can formed into fibers by conventional processes such as spinning techniques, including melt, solution, dry, gel, and the like. Methods for spinning fibers from copolymers and polymers are well known in the art and will not be discussed further herein.

The molecular weight of the random copolymer can vary widely depending upon the use of the copolymer formed. In general, the molecular weight of the copolymer is sufficiently high to allow its use in the fabrication of medical devices. Useful average molecular weight ranges of the copolymers for use in any particular situation will vary depending upon such features as the ultimate fiber properties and characteristics desired, such as modulus, tensile strength, bioresorption and biodegradation rates, and the like. In general, copolymer molecular weights useful for forming fibers are equal to or greater than about 10,000. Suitable average molecular weight ranges are about 10,000 to about 5,000,000, or about 20,000 to about 1,000,000, or about 30,000 to about 500,000.

Other polymeric components such as fillers and binders can be combined with the copolymers prior to and/or during the formation of fibers or devices, or subsequent to their formation. Suitable fillers and binders are known and will not be discussed further herein.

In addition, other degradable polymeric systems can be used according to the invention, such as polysaccharides and polypeptides. One of skill in the art, upon review of this disclosure, will readily appreciate the application of the inventive concepts to these additional degradable polymeric materials.

The biodegradable compositions are composed of at least one of the biodegradable polymers described herein, namely polyetherester copolymers (such as PEGT/PBT), terephthalate esters with phosphorus-containing linkages, and segmented copolymers with differing ester linkages, or polycarbonate-containing random copolymers. Optionally, the biodegradable composition further includes one or more bioactive agents.

Selection of the biodegradable polymer can be impacted by one or more considerations, such as, for example, the bioactive agent release rate desired for a particular application, the hydrophobicity of the polymer or polymers, and solvent compatibility. As an initial step, a bioactive agent is selected for treatment. Next a release rate that would provide a therapeutic or prophylactic dosage of the bioactive agent to a patient can be determined, based upon (for example) many of the considerations mentioned herein. Once a biodegradable composition release rate is determined, this rate can be utilized to establish parameters for selection of the biodegradable polymer system to be utilized in fabricating the device.

The bioactive agent release rate can be modulated in a number of ways. In some aspects, the relative amounts of biodegradable polymer(s) to bioactive agent(s) can be adjusted to further modulate the bioactive agent release rate. In some aspects, the composition of the biodegradable copolymer can be modified to modulate release rate. For example, when the biodegradable copolymer comprises an amphiphilic copolymer having hydrophilic units and hydrophobic units, the proportion of faster degrading polymer components (such as hydrophilic units) can be increased relative to the slower degrading polymer components (such as hydrophobic units) to provide a faster biodegradable composition release rate. In some embodiments, when most of the bioactive agent dosage is desired to be released over a long time period, the proportion of slower releasing polymer component can be increased relative to the faster releasing polymer component within the biodegradable copolymer.

Another selection parameter for the biodegradable polymer can be solvent compatibility. In some aspects, the solvent system for the biodegradable polymer(s) and bioactive agent(s) are compatible.

The principle mode of degradation for many of the biodegradable polymers is hydrolysis. Degradation proceeds first by diffusion of water into the material followed by random hydrolysis, fragmentation of the material, and finally a more extensive hydrolysis accompanied by phagocytosis, diffusion, and metabolism. The hydrolysis can be affected by the size and hydrophilicity of the particular polymer material, the crystallinity of the polymer, and the pH and temperature of the environment. Once the polymer is hydrolyzed, the products of hydrolysis are either metabolized or secreted.

Suitable solvents that can be used to formulate the biodegradable composition include, but are not limited to, chloroform, water, alcohol, acetone, acetonitrile, ether, methyl ethyl ketone (MEK), ethyl acetate, tetrahydrofuran (THF), dioxane, methylene chloride, xylene, toluene, N,N-dimethylformamide (DMF), dimethyl sulfoxide (DMSO), N,N-dimethylacetamide (DMAC), N-methylpyrrolidone (NMP), combinations of these, and the like.

To form biodegradable composition with bioactive agent, the selected biodegradable polymers are combined and mixed with a bioactive agent. The bioactive agent can be present as a liquid, a finely divided solid, or any other appropriate physical form. The variety of different bioactive agents that can be used in conjunction with the biodegradable polymers of the invention is vast. The inventive biodegradable compositions can find particular utility for delivery of small molecular weight bioactive agents, as described herein. Optionally, the biodegradable composition can include one or more additives, such as diluents, carriers, excipients, stabilizers, or the like.

Upon contact with body fluids, the biodegradable composition undergoes gradual degradation (mainly through hydrolysis) with concomitant release of the bioactive agent for a sustained or extended period. Bioactive agent release can be attributed to diffusion of the bioactive agent through the polymer matrxi, and/or degradation of the polymer matrix. This can result in prolonged delivery (such as a period of several weeks) of therapeutically or prophylactically effective amounts of the bioactive agent. The therapeutically and/or prophylactically effective amount can be determined based upon such factors as the patient being treated, the severity of the condition, the judgment of the prescribing physician, and the like. In light of the teaching herein, those skilled in the art will be capable of preparing a variety of formulations.

In still further aspects, the composition of the copolymers themselves can be manipulated to provide desirable features. For example, when the copolymers include hydrophobic and hydrophilic portions, the relative amounts of these portions can be varied within the copolymer to provide a particular degradation rate. Likewise, the relative amounts of these portions can be varied within the copolymer to provide a desired bioactive agent release rate. It will be readily appreciated that bioactive agent release rate can be impacted by the degradation rate of the polymer, as well as the ability of the bioactive agent to diffuse from the polymer. Also, the ability for liquids (such as aqueous fluids) to permeate the polymer can impact the bioactive agent release rate and/or degradation rate. The present description provides various degradable polymer systems that can be utilized to deliver bioactive agent to intraluminal treatment sites, such as intravascular or extravascular sites. It will be appreciated that these illustrative degradable polymer systems can be manipulated to adjust bioactive release rate and/or degradation rate of the polymer.

Thus, the invention provides implantable intraluminal devices (such as stents) that are fabricated of a polymeric material. The polymeric material can be selected from polymers containing ester linkages (such as polyetherester copolymers, terephthalate esters with phosphorus-containing linkages, and segmented copolymers with differing ester linkages), or polycarbonate-containing random copolymers. Optionally, the polymer material can include one or more bioactive agents, thereby providing a drug-delivery device. These drug-delivery embodiments will now be described in more detail.

In some aspects of the invention, the polymeric material includes a bioactive agent. As used herein, “bioactive agent” refers to an agent that affects physiology of biological tissue. Bioactive agents useful according to the invention include virtually any substance that possesses desirable therapeutic and/or prophylactic characteristics for application to the implantation site.

For ease of discussion, reference will repeatedly be made to a “bioactive agent.” While reference will be made to a “bioactive agent,” it will be understood that the invention can provide any number of bioactive agents to a treatment site. Thus, reference to the singular form of “bioactive agent” is intended to encompass the plural form as well. Moreover, for purposes of discussion, reference will be made to association of the bioactive agent with a polymeric material composed of PEGT/PBT. However, it will be apparent upon review of this disclosure that the bioactive agent can be associated with any of the polymeric systems described herein. Further, the additives described herein are applicable to all polymer systems disclosed as well.

Exemplary bioactive agents include, but are not limited to, thrombin inhibitors; antithrombogenic agents; thrombolytic agents (such as plasminogen activator, or TPA: and streptokinase); fibrinolytic agents; vasospasm inhibitors; calcium channel blockers; vasodilators; antihypertensive agents; clotting cascade factors (for example, protein S); anti-coagulant compounds (for example, heparin and nadroparin, or low molecular weight heparin); antimicrobial agents, such as antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate, minocycline, doxycycline, vancomycin, kanamycin, cephalosporins such as cephalothin, cephapirin, cefazolin, cephalexin, cephardine, cefadroxil, cefamandole, cefoxitin, cefaclor, cefuroxime, cefonicid, ceforanide, cefitaxime, moxalactam, cetizoxime, ceftriaxone, cefoperazone), geldanamycin and analogues, antifungals (such as amphotericin B and miconazole), and antivirals (such as idoxuridine trifluorothymidine, acyclovir, gancyclovir, interferon, α-methyl-P-adamantane methylamine, hydroxy-ethoxymethyl-guanine, adamantanamine, 5-iodo-deoxyuridine, trifluorothymidine, interferon, adenine arabinoside); inhibitors of surface glycoprotein receptors; antiplatelet agents (for example, ticlopidine); antimitotics; microtubule inhibitors; anti-secretory agents; active inhibitors; remodeling inhibitors; antisense nucleotides (such as morpholino phosphorodiamidate oligomer); anti-metabolites; antiproliferatives (including antiangiogenesis agents, taxol, sirolimus (rapamycin), analogues of rapamycin (“rapalogs”), tacrolimus, ABT-578 from Abbott, everolimus, paclitaxel, taxane, vinorelbine); anticancer chemotherapeutic agents; anti-inflammatories (such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide); non-steroidal anti-inflammatories (such as salicylate, indomethacin, ibuprofen, diclofenac, flurbiprofen, piroxicam); antiallergenics (such as sodium chromoglycate, antazoline, methapyriline, chlorpheniramine, cetrizine, pyrilamine, prophenpyridamine); anti-proliferative agents (such as 1,3-cis retinoic acid); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); mydriatics (such as atropine, cyclopentolate, homatropine, scopolamine, tropicamide, eucatropine, hydroxyamphetamine); sympathomimetics (such as epinephrine); antineoplastics (such as carmustine, cisplatin, fluorouracil); immunological drugs (such as vaccines and immune stimulants); hormonal agents (such as estrogens, estradiol, progesterol, progesterone, insulin, calcitonin, parathyroid hormone, peptide and vasopressin hypothalamus releasing factor); beta adrenergic blockers (such as timolol maleate, levobunolol HCl , betaxolol HCl); immunosuppressive agents, growth hormone antagonists, growth factors (such as epidermal growth factor, fibroblast growth factor, platelet derived growth factor, transforming growth factor beta, somatotropin, fibronectin, insulin-like growth factor (IGF)); carbonic anhydrase inhibitors (such as dichlorophenamide, acetazolamide, methazolamide); inhibitors of angiogenesis (such as angiostatin, anecortave acetate, thrombospondin, anti-VEGF antibody such as anti-VEGF fragment—ranibizumab (Lucentis)); dopaamine agonists; radiotherapeutic agents; peptides; proteins; enzymes; nucleic acids and nucleic acid fragments; extracellular matrix components; ACE inhibitors; free radical scavengers; chelators; antioxidants; anti-polymerases; photodynamic therapy agents; gene therapy agents; and other therapeutic agents such as prostaglandins, antiprostaglandins, prostaglandin precursors, and the like.

Another group of useful bioactive agents are antiseptics. Examples of antiseptics include silver sulfadiazine, chlorhexidine, glutaraldehyde, peracetic acid, sodium hypochlorite, phenols, phenolic compounds, iodophor compounds, quaternary ammonium compounds, and chlorine compounds.

Another group of useful bioactive agents are enzyme inhibitors. Examples of enzyme inhibitors include chrophonium chloride, N-methylphysostigmine, neostigmine bromide, physostigmine sulfate, tacrine HCL, tacrine, 1-hydroxymaleate, iodotubercidin, p-bromotetramisole, 10-(α-diethylaminopropionyl)-phenothiazine hydrochloride, calmidazoliurn chloride, hemicholinium-3,3,5-dinitrocatechol, diacylglycerol kinase inhibitor 1, diacylglycerol kinase inhibitor II, 3-phenylpropargylamine, N-monomethyl-L-arginine acetate, carbidopa, 3-hydroxybenzylhydrazine HCl, hydralazine HCl, clorgyline HCl, deprenyl HCl, L(−)deprenyl HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-diphenylvalerate hydrochloride, 3-isobutyl-1-methylxanthine, papaverine HCl, indomethacin, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate, R(+) p-aminoglutethimide tartrate, S(−)3-iodotyrosine, alpha-methyltyrosine, L(−)alpha methyltyrosine, D,L(−)cetazolamide, dichlorophenamide, 6-hydroxy-2-benzothiazolesulfonamide, and allopurinol.

Another group of useful bioactive agents are anti-pyretics and antiinflammatory agents. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide. Local anesthetics are substances that have an anesthetic effect in a localized region. Examples of such anesthetics include procaine, lidocaine, tetracaine and dibucaine.

Biodegradable compositions can be formulated by mixing one or more bioactive agents with the polymers. The bioactive agent can be present as a liquid, a finely divided solid, or any other appropriate physical form. Typically, but optionally, the biodegradable composition will include one or more additives, such as diluents, carriers, excipients, stabilizers, or the like.

The particular bioactive agent, or combination of bioactive agents, can be selected depending upon one or more of the following factors: the application of the device (for example, coronary stent, graft, or the like), the amount of the device composed of the polymeric material (for example, percentage of the device fabricated of degradable material, inclusion of a biodegradable material as a coating on a surface of the body member, as well as the amount of surface provided with the coating), the medical condition to be treated, the anticipated duration of treatment, characteristics of the implantation site, the number and type of bioactive agents to be utilized, and the like.

The concentration of the bioactive agent in the polymeric material can be provided in the range of about 0.01% to about 75% by weight, or about 0.01% to about 50% by weight, based on the weight of the final polymeric material. In some aspects, the bioactive active agent is present in the polymeric material in an amount in the range of about 75% by weight or less, or in the range of about 50% by weight or less. The amount of bioactive agent in the polymeric material can be in the range of about 1 μg to about 10 mg, or about 100 μg to about 1000 μg, or about 300 μg to about 600 μg.

In some aspects, the concentration of bioactive agent can also be selected to provide a desired elution rate from the device. As discussed herein, some aspects of the invention provide methods including steps of selecting one or more bioactive agents to administer to a patient, determining a treatment course for a particular patient, and formulating the polymeric material to achieve the treatment course.

The inventive implants can be utilized to deliver any desired bioactive agent or combination of bioactive agents to a treatment site, such as the bioactive agents described herein. The amount of bioactive agent(s) delivered over time is preferably within the therapeutic level, and below the toxic level. In some aspects, the treatment course is greater than 4 weeks, or greater than 6 weeks, or greater than 8 weeks, or greater than 10 weeks, or greater than 3 months, or greater than 6 months, or greater than one year. Thus, in preferred embodiments, the bioactive agent is released from the coated composition in a therapeutically effective amount for a period of 4 weeks or more, or 6 weeks or more, or 8 weeks or more, or 10 weeks or more, or 3 months or more, or 6 months or more, or 9 months or more, or 12 months or more, or 36 months or more, when implanted in a patient.

The inventive implants are formulated and configured to degrade upon implantation for a degradation period. Optionally, the implants also release bioactive agent in a controlled manner for a release period. Generally speaking, the degradation period is longer than the bioactive agent. Put another way, the inventive implants release bioactive agent for a selected amount of time within the degradation period. In some aspects, the bioactive agent release period is 75% or less of the degradation period, or 70% or less of the degradation period, or 60% or less of the degradation period, or 50% or less than the degradation period, or 40% or less of the degradation period, or 30% or less of the degradation period, or 25% or less of the degradation period, or 20% or less of the degradation period. As mentioned, the degradation period comprises a longer period of time, relative to the bioactive agent release period. In some aspects, the degradation period comprises the amount of time a significant amount of the implant remains intact within the body (such as the amount of time a detectable, intact portion of the initial implant can be found at the implantation site). In some embodiments, the degradation period is 3 years or less, or 2 years or less, or 1 year or less, or 6 months or less. In some embodiments, the degradation period is in the range of 0.5 to 2 years.

In some aspects, the concentration of bioactive agent can be selected to provide a desired tissue concentration of bioactive agent at the treatment site. Given the site-specific nature of the inventive devices, methods and systems, it will be apparent that the tissue concentration of bioactive agent will be greater at the treatment site than at areas within the patient outside the treatment site. As discussed herein, this provides several benefits to the patient, such as reduced risk of toxic levels of the bioactive agent within the body, reduced risk of adverse affects caused by bioactive agent outside the treatment site, and the like. The location of the bioactive agent on or within the device and on or within the polymer can also affect tissue concentration of bioactive agent (for example, when substantially the entire device body includes bioactive agent, or selected portion(s) of the device body include bioactive agent). Moreover, inclusion of optional coating layers that contain bioactive agent can also impact tissue concentration of bioactive agent.

In one illustrative embodiment, when a relatively small-sized bioactive agent (for example, many antimicrobial agents, antiviral agents, and the like) is included in a PEGT/PBT polymeric material, the polyethylene glycol component of the copolymer preferably has a molecular weight in the range of about 200 to about 10,000, or in the range of about 300 to about 4,000. Also, the polyethylene glycol terephthalate is preferably present in the copolymer in an amount in the range of about 30 weight percent to about 80 weight percent of the weight of the copolymer, or in the range of about 50 weight percent to about 60 weight percent of the weight of the copolymer. According to these particular embodiments, the polybutylene terephthalate is present in the copolymer in an amount in the range of about 20 weight percent to about 70 weight percent of the copolymer, or in the range of about 40 weight percent to about 50 weight percent of the copolymer.

The inventive implants are formulated and configured to degrade upon implantation for a degradation period, and to release bioactive agent in a controlled manner for a release period. Generally speaking, the degradation period is longer than the bioactive agent. Put another way, the inventive implants release bioactive agent for a selected amount of time within the degradation period. In some aspects, the bioactive agent release period is 75% or less of the degradation period, or 70% or less of the degradation period, or 60% or less of the degradation period, or 50% or less than the degradation period, or 40% or less of the degradation period, or 30% or less of the degradation period, or 25% or less of the degradation period, or 20% or less of the degradation period. As mentioned, the degradation period comprises a longer period of time, relative to the bioactive agent release period. In some aspects, the degradation period comprises the amount of time a significant amount of the implant remains intact within the body (such as the amount of time a detectable, intact portion of the initial implant can be found at the implantation site). In some embodiments, the degradation period is 3 years or less, or 2 years or less, or 1 year or less, or 6 months or less. In some embodiments, the degradation period is in the range of 0.5 to 2 years.

Additives

In some aspects, it can be desirable to provide one or more additives to the biodegradable polymer material. Such additives can be particularly desirable when bioactive agent is included in the polymer. Additives can be included to impact the release of bioactive agent from the device. Suitable additives according to these aspects include, but are not limited to, hydrophobic antioxidants, hydrophobic molecules, and hydrophilic antioxidants. Alternatively, additives can be included to impact imaging of the device once implanted. Illustrative additives will now be described in more detail.

In some embodiments, the polymeric material can optionally include at least one hydrophobic antioxidant. For example, when the polyetherester material (such as PEGT/PBT) includes a hydrophobic small-sized drug (such as, for example, a steroid hormone), the polymeric material can include at least one hydrophobic antioxidant. Exemplary hydrophobic antioxidants that can be employed include, but are not limited to, tocopherols (such as α-tocopherol, β-tocopherol, γ-tocopherol, δ-tocopherol, ε-tocopherol, zeta₁-tocopherol, zeta₂-tocopherol, and eta-tocopherol), and ascorbic acid 6-palmitate. Such hydrophobic antioxidants can retard the degradation of the polyetherester copolymer material, and/or retard the release of the bioactive agent contained in the polymeric material. Thus, the use of a hydrophobic or lipophilic antioxidant can be desirable particularly to the formation of polymeric materials that include drugs that tend to be released quickly from the polymer, such as, for example, small drug molecules having a molecular weight less than 500 (in other words, the use of a hydrophobic or lipophilic antioxidant can slow release of the drug from the polymer if desired). In some embodiments, the antioxidant can improve drug stability as well. For example, inclusion of rapamycin in drug eluting stents (“DES”) can be problematic, as rapamycin can be less stable than desired. Thus, inclusion of a hydrophobic antioxidant can, in some embodiments, improve the stability of rapamycin in a bioactive agent delivery device.

Typically, the hydrophobic antioxidant(s) can be present in the polymeric material in an amount up to about 10 weight percent, or in the range of about 0.1 weight percent to about 10 weight percent of the total weight of the polymeric material, or in the range of about 0.5 weight percent to about 2 weight percent.

In some embodiments, the polymeric material can optionally include one or more hydrophobic molecules. For example, when the polyetherester material includes a hydrophilic small-size drug (for example an aminoglycoside such as gentamycin), the polymer material can also include, instead of, or in addition to, the hydrophobic antioxidant herein described, at least one hydrophobic molecule. Illustrative hydrophobic molecules useful with the polymeric material include cholesterol, ergosterol, lithocholic acid, cholic acid, dinosterol, betuline, and/or oleanolic acid. One or more hydrophobic molecules can act to retard the release rate of the bioactive agent from the polyetherester copolymer. Such hydrophobic molecules can prevent water penetration into the polymer material, but do not compromise the degradability of the polymeric material. In addition, such molecules have melting points in the range of 150° C. to 200° C. or more. Therefore, a small percentage of these molecules increase the Tg of the polymeric material, which decreases the matrix diffusion coefficient for the bioactive agent to be released. Thus, such hydrophobic molecules can, in some embodiments, provide for a more sustained release of a bioactive agent from the polymeric material.

The hydrophobic molecule(s) can be present in the polymeric material in an amount up to about 20 weight percent, or in the range of about 0.1 weight percent to about 20 weight percent, or about 1 weight percent to about 5 weight percent.

When the polyetherester copolymer contains a protein, the copolymer can also optionally include a hydrophilic antioxidant. Examples of hydrophilic antioxidants include, but are not limited to, those having the following structural formula XXXII:
(X₁)_{Y}A-(X₂)_{Z} XXXII
wherein each of Y and Z is 0 or 1, wherein at least one of Y and Z is 1. Each of X₁ and X₂ is independently selected from the group consisting of compounds of the formula XXXIII: 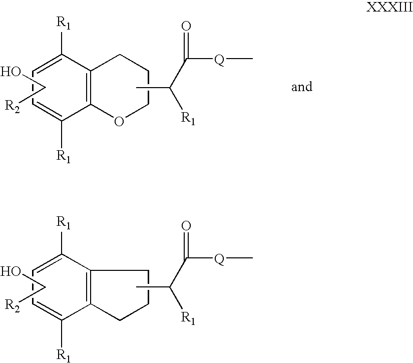
wherein each R₁ is hydrogen or an alkyl group having 1 to 4 carbon atoms, preferably methyl, and each R₁ is the same or different. R₂ is hydrogen or an alkyl group having 1 to 4 carbon atoms, preferably methyl. Q is NH or oxygen. Each of X₁ and X₂ can be the same or different. A is:
—(—R₃—O)ₙ—R₄ XXXIV
wherein R₃ is an alkyl group having 1 or 2 carbon atoms, preferably 2 carbon atoms; n is 1 to 100, preferably from 4 to 22; R₄ is an alkyl group having 1 to 4 carbon atoms, preferably 1 or 2 carbon atoms.

In one embodiment, one of Y and Z is 1, and the other of Y and Z is 0. In another embodiment, each of Y and Z is 1.

In yet another embodiment, R₃ is ethyl.

In a further embodiment, R₄ is methyl or ethyl.

In yet another embodiment, R₁ is methyl, R₂ is methyl, R₃ is ethyl, R₄ is methyl, one of Y and Z is 1 and the other of Y and Z is 0, Q is NH, n is 21 or 22, and the antioxidant has the following structural formula XXXV: 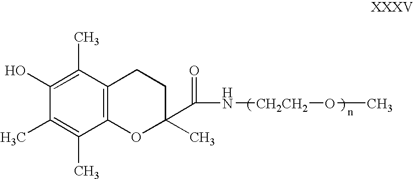
In another embodiment, the hydrophilic antioxidant has the following structural formula:
(X₃)_{Y}-A-(X₄)_{Z} XXXVI
wherein each of Y and Z is 0 or 1, wherein at least one of Y and Z is 1. Each of X₃ and X₄ is: 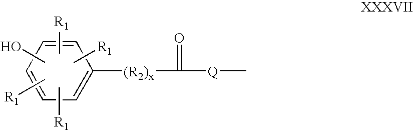
wherein each R₁ is hydrogen or an alkyl group having 1 to 4 carbon atoms, R₂ is an alkyl group having 1 to 4 carbon atoms, x is 0 or 1, and Q is NH or oxygen. Each R₁ is the same or different, and each of the X₃ and X₄ is the same or different. A is:
—(R₃—O—)ₙ—R₄ XXXVIII
wherein R₃ is an alkyl group having 1 or 2 carbon atoms, preferably 2 carbon atoms; n is from 1 to 100, preferably from 4 to 22; and R₄ is an alkyl group having 1 to 4 carbon atoms, preferably 1 or 2 carbon atoms.

In one embodiment, at least one, preferably two, of the R₁ moieties is a tert-butyl moiety. When two of the R₁ moieties are tert-butyl moieties, each tert-butyl moiety is preferably adjacent to the —OH group.

The hydrophilic antioxidant(s) can be present in the polymeric material in an amount up to about 10 weight percent, or in the range of about 0.1 weight percent to about 10 weight percent, or about 1 weight percent to about 5 weight percent, based upon the total weight of the polymeric material.

As discussed herein, the polymeric material can include a hydrophobic antioxidant, hydrophobic molecule, and/or a hydrophilic antioxidant in the amounts described herein. The type and precise amount of antioxidant and/or hydrophobic molecule employed can be dependent upon the molecular weight of the bioactive agent (protein), as well as properties of the polymeric matrix itself. If the polymeric material includes a large peptide or protein (such as, for example, insulin), the matrix can also optionally include a hydrophilic antioxidant such as those described herein and in the amounts described herein, and can also include polyethylene glycol having a molecular weight in the range of about 1,000 to about 4,000, in an amount in the range of about 1 weight percent to about 10 weight percent, based upon the total weight of the copolymer.

Additives—Imaging Materials

In some embodiments, the polymer material can further include imaging materials. For example, materials can be included in the polymer material to assist in medical imaging of the device once implanted. Medical imaging materials are well known. Exemplary imaging materials include paramagnetic material, such as nanoparticular iron oxide, Gd, or Mn, a radioisotope, and non-toxic radio-opaque markers (for example, cage barium sulfate and bismuth trioxide). Radiopacifiers (such as radio opaque materials) can be included in any fabrication method or absorbed into or sprayed onto the surface of part or all of the implant. The degree of radiopacity contrast can be altered by controlling the concentration of the radiopacifier within or on the implant. Radiopacity can be imparted by covalently binding iodine to the polymer monomeric building blocks of the elements of the implant. Common radio opaque materials include barium sulfate, bismuth subcarbonate, and zirconium dioxide. Other radio opaque materials include cadmium, tungsten, gold, tantalum, bismuth, platinum, iridium, and rhodium. In some embodiments, iodine can be employed for both its radiopacity and antimicrobial properties. This can be useful for detection of medical devices that are implanted in the body (that are emplaced at the treatment site) or that travel through a portion of the body (that is, during implantation of the device). Paramagnetic resonance imaging, ultrasonic imaging, x-ray means, fluoroscopy, or other suitable detection techniques can detect medical devices including these materials. In another example, microparticles that contain a vapor phase chemical can be used for ultrasonic imaging. Useful vapor phase chemicals include perfluorohydrocarbons, such as perfluoropentane and perfluorohexane, which are described in U.S. Pat. No. 5,558,854 (Issued 24 Sep., 1996); other vapor phase chemicals useful for ultrasonic imaging can be found in U.S. Pat. No. 6,261,537 (Issued 17 Jul., 2001).

Thus, additives can be included in the polymer to control release of bioactive agent, impact degradation of the polymer, and/or impact imaging of the device once implanted. In some aspects, release of bioactive agent can also be impacted by modification of the polymer material itself. Another technique for impacting release of bioactive agent can involve modifying the configuration of the device.

Additives—Excipients

In some aspects, one or more polymers comprising the bioactive agent delivery system can include an excipient. A particular excipient can be selected based upon its melting point, solubility in a selected solvent (such as a solvent that dissolves the polymer and/or the bioactive agent), and the resulting characteristics of the composition. Excipients can comprises a few percent, about 5%, 10%, 15%, 20%, 25%, 30%, 40%, 50%, or higher percentage of the particular polymer in which it is included.

Buffers, acids, and bases can be incorporated in the polymer or polymers to adjust their pH. Agents to increase the diffusion distance of bioactive agents released from the polymer matrix can also be included. Illustrative excipients include salts, PEG or hydrophilic polymers, and acidic compounds.

Thus, additives can be included in one or more polymers comprising the biodegradable composition to assist in controlling release of bioactive agent, impacting degradation of the biodegradable composition, and/or impacting imaging of the device once implanted.

Optionally, the copolymer itself can be modified to affect the degradation rate and release rate of a bioactive agent. For example, the copolymer can be modified by replacing components (monomeric units) with a particular hydrophobicity with a component (monomeric unit) that has a differing hydrophobicity.

For example, with PEGT/PBT, when a protein having a molecular weight greater than 10,000 is contained within the polyethylene glycol terephthalate/polybutylene terephthalate copolymer, the polyethylene glycol component of the copolymer can have a molecular weight in the range of about 300 to about 10,000. The polyethylene glycol terephthalate can be present in the copolymer in an amount in the range of about 30 weight percent to about 90 weight percent, or about 50 weight percent to about 85 weight percent of the weight of the copolymer. The polybutylene terephthalate can be present in the copolymer in an amount in the range of about 10 weight percent to about 70 weight percent, or about 15 weight percent to about 50 weight percent of the weight of the copolymer.

These concepts can be applied to the other degradable polymer systems described herein as well. The composition of the copolymer can be modified whether additives are included in the copolymer or not.

The inventive biodegradable devices comprise a body member component comprising a biodegradable polymeric material selected to provide mechanical properties to the device. Features of the body member component will now be described in more detail.

Generally speaking, the long-term residence of stents can present long-term risks and complications. A stent can provide one or a combination of the following characteristics: (1) mimics the tissue it is designed to replace in size, shape, and material consistency; (2) is unlikely to induce infection or trigger a foreign body response; (3) is a temporary prosthesis that takes on characteristics of the natural tissue as it degrades; and (4) is a biocompatible implant that has a smooth surface to minimize risk for thrombus formation and macrophage enzyme activity.

Referring to a specific application as illustrative of aspects of the invention, degradable stents can provide a number of advantages. Such stents that are capable of integrating seamlessly with the living host tissue can improve tissue biocompatibility due to their temporary residence. With the initial strength to secure the diseased or damaged tissue, such stents can eliminate the concern for implant migration over time and long-term implant failure. They can also minimize time, costs, and complications associated with re-intervention of specific and neighboring sites.

In-stent restenosis is thought to be a consequence of a number of factors, such as injury to the vessel wall at implantation, thrombosis formation, and/or tissue hyperplasia, occurring principally at the points where the stent's struts impinge upon the artery wall. Placement of an excessively stiff stent against a compliant vessel wall creates a mismatch in mechanical behavior that can result in continuous lateral expansile stress on the vessel wall. This stress can promote thrombosis, arterial wall thinning, or excessive cellular proliferation. Hence, it is believed that polymeric biomaterials, which can be more flexible than traditional metal stents, may minimize the pathology and are more likely to approximate the mechanical profile of the native tissue.

In some aspects, materials selected for the implantable intraluminal body member can provide one or more of the following features. In some aspects, the polymeric material of the body member is selected to provide mechanical support for the desired amount of time, after which the stent material can degrade. The materials can provide one or more of the following features: (1) resist failure due to the multiaxial stress-strain behavior of native arteries; (2) retain mechanical strength during several weeks or months post-deployment; (3) degrade via hydrolytic or enzymatic degradation preferably with surface erosion whereby the stent degrades uniformly and maintains its original shape as its degrades; (4) maintain favorable hemodynamics (reduced turbulent flow); (5) exhibit a smooth and uniform surface; and (6) support endothelialization.

In some aspects, the polymeric material of the body member is selected to provide a Young's modulus similar to that of currently used metal stents. Generally, a human blood vessel has a Young's modulus of approximately 3×10⁷ Pascal. In some aspects, the stent can be designed to have a Young's modulus sufficient to holding the blood vessel in an expanded state, for example, approximately 3×10⁹ or 3×10⁸ Pascal. In other aspects, the stent can be designed to have a differential Young's modulus about the length of the stent, such that a main mid portion of the stent can have a Young's modulus of approximately 3×10⁹ or 3×10⁸ Pascal, while lower tenacity portions can be provided towards the ends of the stent, where the Young's modulus more closely approximates that of the blood vessel (3×10⁷ Pascal). Such variable tenacity can be provided by varying the thickness or density of the material forming the stent along the length (for example, providing a thicker material in the main mid portion of the stent, and thinner material at the ends of the stent). In U.S. Pat. No. 6,200,335 (Igaki, Mar. 13, 2001), it is taught that such variable tenacity of the stent material can reduce the stress-concentrated portions along the length of the stent, which can contribute to restenosis.

In some aspects, the polymeric material of the body member provides sufficient hoop strength to support the vessel wall against collapse and yet is flexible and compliant enough for safe and effective delivery to the site of a stenotic portion of a vessel. In some aspects, the polymeric material of the body member has sufficient strength to withstand collapse pressures to be encountered upon implantation and use.

In some aspects, the polymeric material of the body member is soft and compliant to avoid arterial rupture or aneurysm formation at the ends of the stent even when exposed to continuous stresses after implantation during residence within a patient. In some aspects, the polymeric material of the body member provides sufficient longitudinal flexibility for ease of insertion and easy expandability, so that it can be expanded inside the vessel and then deployed by suitable expansion means.

Regarding degradation of the polymeric material of the body member, the following features can be provided. In some aspects, the polymeric material of the body member is selected to provide a slower degradation rate relative to the polymeric material of the bioactive agent delivery system. In these aspects, the structural component of the device remains in the patient body after all, or substantially all, of the bioactive agent delivery polymeric material has been degraded by the body. Suitable materials degrade and are absorbed with the production of physiologically acceptable breakdown products and the loss of strength and mass are appropriate to the particular biological environment and clinical function requirements.

Generally speaking, the mechanical properties of polymers increase with increasing molecular weight. For instance, strength and tensile modulus of polymers generally increase with increasing molecular weight. Thus, for biodegradable polymers useful as a structural component herein, increasing molecular weight of the polymers can provide increased strength and tensile modulus, thus enhancing these features of the polymeric material and/or providing additional polymeric materials that can be used as this component of the device.

In some aspects, the polymeric material of the body member is selected to provide a device capable of expansion from a first circumferential diameter to a second diameter upon placement at an implantation site. In some aspects, the second diameter is at least about 5% more than the first diameter, or at least about 25%, or at least about 50%, or at least about 100%, or at least about 200%, or at least about 300%, or at least about 400% more than the first diameter. In some aspects, the second diameter is about 100% to about 400% more than the first diameter. In some aspects, the stent has a first circumferential length before placement at implantation site, and a second circumferential length upon placement at an implantation site. In some aspects, the second circumferential length is at least 5% more than the first circumferential length.

In still further aspects, the polymeric material of the body member is selected to be relatively thin-walled, the particular wall thickness being selected based upon the selected materials and their mechanical properties, typically in the range of less than about 0.006 inches (0.154 mm) for degradable materials described herein. In some aspects, thin walls can also minimize blood turbulence and thus risk of thrombosis. The stent design chosen can also impact the wall thickness, as will be readily appreciated upon review of this disclosure.

The body member of the implantable devices can be formed by any known method for forming polymeric devices such as stents. For example, in one illustrative embodiment, PEGT/PBT copolymer is utilized to fabricate a stent. The stent can be formed by any number of well-known methods including extrusion, such as melt extrusion or solvent extrusion. The extrusion procedure can be varied depending upon the stability of bioactive agent (if any) to be included in the polymer material. In the solvent extrusion method, bioactive agent and polymer solutions are prepared at high concentrations (approximately 1 g/ml), and are forced through a narrow syringe needle. Filament thickness can be varied easily between approximately 150 to 1000 μm. Other suitable fabrication methods include molding (such as injection molding), weaving fibers into the body, and the like.

Preselected patterns of voids can then be formed into the tube in order to define a plurality of spines and struts that impart a degree of flexibility and expandability to the tube. Such patterns can be provided by cutting into the tube using die-cutting, machining or laser cutting. The resulting patterns can assume any shape that does not adversely affect the compression and self-expansion characteristics of the final stent. The pattern can be achieved by forming openings (voids) in the stent material that are of the same size, or openings of different sizes. Providing such openings or voids throughout the material of the stent can allow for sufficient tissue ingrowth between the filaments of the polymer, thereby fixing the stent in position and minimizing the likelihood of stent migration and/or dislodgment.

In another embodiment, stents can be made by subjecting the polymeric melt to extrusion molding to produce filaments having a desired diameter (for example, in the range of 1 to 2 mm). The filaments can be drawn (to induce orientation and self-reinforcement) at a temperature (T) of Tm>T>Tg (where Tg is polymer glass transition temperature and Tm is polymer melting temperature) to a specified diameter (for example, 1 mm). Filaments are then wound in a hot state around a substrate (such as a metal pipe having a diameter of 5 mm), cooled, and removed from the surface of the substrate. The stents are then immersed in buffer solutions, if desired, to maintain pH in a desired range.

In yet another embodiment, a stent is prepared from biodegradable polymer matrix containing biodegradable reinforcing fibers. First, a bundle of fibers with fine particulate polymer powder (particle size in the range of 1 to 10 μm) mixed therein is compression molded in a rod-shaped mold of desired dimensions (diameter and length) above the melting point of the matrix polymer. The reinforcing fibers can compose 10-60%, or 20-60% by volume of the matrix polymer. The rods are then heated and wound helically around a hot cylindrical mold of desired dimensions (diameter and length), and the mold is cooled. The resultant implantable device consists of matrix polymers and fibrous reinforcements.

In some aspects, the body member can be formed by known fiber-forming techniques, such as spinning (including melt spinning and electrospinning). In some aspects, the body member is formed by melt spinning. Spinning from solution can be used in lieu of high temperature (about 190° C.) melt extrusion. Methylene chloride (b.p. 55° C.) is one solvent for use in such a process. The solvent can be removed during the spinning process by: (i) evaporating solvent from the protofibers descending from a spinneret with warm air (dry spinning) or (ii) squirting the polymer solution into a liquid bath, the liquid being a non-solvent for the polymer but miscible with the solvent in the spinning solution, for example, methyl alcohol (wet spinning).

Self-expanding stents can be formed from a plurality of strands of biodegradable material that can be deformed so as to have a reduced diameter which facilitates delivery of the stent to the implantation site and, once disposed at the implantation site, can be allowed to expand to its preformed configuration to dilate and support that portion of the vessel. The stent body can be woven from a plurality of strands of biodegradable material into a braided pattern.

Expandable stents can be delivered to the implantation site in a reduced diameter configuration on the distal end of an expandable catheter and can be expanded in vivo to its supporting diameter by expanding the expandable portion of its associated catheter. An expandable stent can be a mesh type configuration or in the form of a sheet of biocompatible material.

Polymeric stent bodies can be in the form of a pair of sheets of bioabsorbable material which have been interconnected so as to define tine receiving cavities with pieces of a solid bioabsorbable material in the form of plurality of tines interconnected to the tine receiving cavities. In a further embodiment, the stent can be in the form of a rolled up sheet of bioabsorbable material.

When the stent includes multiple component parts or elements, the intraluminal device can be made using hot-stamp embossing to generate the parts and heat-staking to attach linkage elements and coupling arms. Other methods include laser ablation using a screen, stencil or mask; solvent casting, forming by stamping, embossing, compression molding, centripetal spin casting and molding; extrusion and cutting, three-dimensional rapid prototyping using solid free-form fabrication technology, stereolithography, selective laser sintering, or the like; etching techniques comprising plasma etching; textile manufacturing methods comprising felting, knitting, or weaving; molding techniques comprising fused deposition molding, injection molding, room temperature vulcanized (RTV) molding, or silicone rubber molding; casting techniques comprising casting with solvents, direct shell production casting, investment casting, pressure die casting, resin injection, resin processing electroforming, or reaction injection molding (RIM). Parts thus formed can be connected or attached by solvent or thermal bonding, or by mechanical attachment. Other methods of bonding include the use of ultrasonic radiofrequency or other thermal methods, and by solvents or adhesives or ultraviolet curing processes or photoreactive processes. The elements can be rolled by thermal forming, cold forming, solvent weakening forming and evaporation, or by performing parts before linking.

In some aspects, the biodegradable implantable devices can include a biodegradable coating on at least a surface of the device body. Typically, biodegradable coatings are provided to a surface of the body member after fabrication of the body member. In this way, stability and activity of the bioactive agent activity can preferably be protected from the conditions of fabrication of the structural portion of the device (conditions such as heat, pressure, and the like).

The coatings of the invention can be applied to a surface in a manner sufficient to provide a suitably durable and adherent coating on the surface. Typically, the coatings are provided in a manner such that they are not chemically bound to the surface. Rather, the coatings can be envisioned as encapsulating the device surface. Given the nature of the association between the coating and surface, it will be readily apparent that the coatings can be applied to virtually any surface material to provide a suitably durable and adherent coating. Moreover, in some embodiments, a suitable surface pretreatment can be utilized, to enhance the association between the coating and the device surface.

In some embodiments, the stent can be immersed in a biodegradable composition solution to form a coating. In other embodiments, the biodegradable coating composition is spray coated onto a surface of an implantable device. Alternatively, the biodegradable coating composition can be extruded in the form of a tube that is then codrawn over a tube of material comprising the body member. By codrawing two tubes of the biodegradable coating composition over the body member, one positioned about the exterior of the body member and another positioned within such body member, a tube having multi-layered walls can be formed. Subsequent perforation of the tube walls to define a preselected pattern of spines and struts can impart the desired flexibility and expandability to the tube to create a stent.

The inventive biodegradable coating compositions can be applied to any desired portion of the device surface. For example, in some embodiments, the biodegradable coating composition can be provided on the entire surface of the device. In other embodiments, only a portion of the device can include the biodegradable coating composition. The portion of the device carrying the biodegradable coating composition can be selected based upon such factors as the application of the device, the amount of bioactive agent to be applied at a treatment site, the number and types of bioactive agents to be delivered, and like factors.

Moreover, each coated layer of the biodegradable coating composition can be provided on the surface of the device in any number of applications. The number of applications can be selected to provide individual coated layers of suitable thickness, as well as a desired total number of multiple coated layers of biodegradable composition, as desired. In such embodiments, the composition of individual layers of the coating can be the same or different, as desired. In some embodiments, the number of applications can be controlled to provide a desired overall thickness to the polymer coating. Generally, the thickness of the coating is selected so that it does not significantly increase the profile of the device for implantation and use within a patient. Typically, the overall thickness of the biodegradable coating composition is on the order of about 1 μm to about 100 μm.

When the implantable devices of the invention include a coating, the coating can be provided with the same or different bioactive agent or agents as the body member of the device. Moreover, when the coating is composed of multiple layers of degradable polymeric material, each individual layer, or groupings of layers, can include different bioactive agents. For example, in a coronary stent, a coating can include an antithrombogenic agent (such as heparin, coumadin and the like) to mitigate acute thrombosis concerns, an inner layer with anti-proliferation agent to prevent sub-acute restenosis issues (for example, everolimus, sirolimus, angiopeptin, paclitaxel, and the like) or anti-inflammatory agent (such as aspirin, lipid lowering statins, fat lowering lipostabil, estrogen and progestin, endothelin receptor antagonist, interleukin-6 antagonist, monoclonal antibodies to VCAM or ICAM, and the like), and the body member material can include growth factors or angiogenesis agent to promote chronic endothelialization at the vessel lumen.

In one embodiment, the inventive concepts can be used to fabricate stents, e.g., either self-expanding stents or expandable stents (such as balloon-expandable stents).

Devices that are particularly suitable include vascular stents such as self-expanding stents and balloon expandable stents. “Expandable” means the stent can be expandable from a reduced diameter configuration utilizing an expansion member, such as a balloon. The particular configuration of the stent body is not critical to the invention described herein, and the inventive biodegradable materials and methods can be applied to virtually any stent configuration.

It can be desirable to fabricate the stent such that the material is nonsolid. In other words, desirable to include pores or other passages through the material that can enable endothelial cells at the implantation site to grow into and over the stent so that biodegradation will occur within the vessel wall rather than in the lumen of the vessel, which could lead to embolization of the dissolved material.

Use

In some aspects, the invention provides methods of treating a lumen within the body. The method includes inserting a polymeric device at an implantation site (an intraluminal site, such as within a blood vessel or other passageway) within a patient. The step of inserting the polymeric device can utilize a catheter, such as those typically utilized for implantation of stents. The catheter can include a balloon or other inflatable member, in the case of expandable stents. The catheter is delivered into a lumen within the patient's body, to the site of an obstruction (or other disorder to be treated), typically utilizing a guidewire.

Once the device (with catheter) has reached the implantation site, the stent is expanded to contact the lumen walls. The stent can be expanded simultaneously with the widening of the obstructed region. After expansion to the desired diameter, the stent remains implanted in the lumen to resist vessel recoil and reduce restenosis, while the catheter and other devices utilized for delivery of the stent are removed from the patient.

In use, the implantable device is placed within a patient at a desired implantation site. Upon contact with body fluids, the body fluids initially permeate at least a portion of the biodegradable composition, allowing for dissolution and diffusion of the bioactive agent from the biodegradable composition. The biodegradable composition undergoes gradual degradation (usually primarily through hydrolysis) with concomitant release of the dispersed bioactive agent for a sustained or extended period.

The device can be fabricated to have a degradation period suitable for the intended device use (treatment). For example, when the device does not include bioactive agent, the degradation period can be determined based upon the period of time mechanical support is desired at the implantation site. In some aspects, the degradation period is on the order of weeks to years. In some aspects, the degradation period is up to 5 years, or up to 4 years, or up to 3 years, or up to 2 years, or up to 1 year, or up to 0.5 years. In some aspects, the degradation period can be 2 weeks or more, or 4 weeks or more, or 6 weeks or more, or 8 weeks or more, or 12 weeks or more. In some aspects, the degradation period is in the range of 0.5 to 2 years.

In some embodiments, when the device includes bioactive agent, the degradation period can be longer than the period during which bioactive agent is released (the bioactive agent release period). Put another way, the overall device can be fabricated to deliver bioactive agent for a release period that is less than the degradation period. In some aspects, the bioactive agent release period can be 50% or less of the degradation period, or 40% or less, or 30% or less, or 25% or less, or 20% or less, or 10% or less. The inventive devices and systems can result in prolonged delivery of therapeutically and/or prophylactically effective amounts of the bioactive agent.

The stents are adapted for deployment and implantation using conventional methods known in the art and employing percutaneous transluminal catheter devices. The stents are designed for deployment by any of a variety of in situ expansion means, such as an inflatable balloon or a polymeric plug that expands upon application of pressure. For example, the tubular body of the stent can be positioned to surround a portion of an inflatable balloon catheter. The stent, with the balloon catheter inside is configured at a first, collapsed diameter. The stent and the inflatable balloon are percutaneously introduced into a body lumen, following a previously positioned guidewire in an over-the-wire angioplasty catheter system, and tracked by suitable means (such as fluoroscopy) until the balloon portion and associated stent are positioned within the body passageway at the implantation site. Thereafter, the balloon is inflated and the stent is expanded by the balloon portion from the collapsed diameter to a second expanded diameter. After the stent has been expanded to the desired final expanded diameter, the balloon is deflated and the catheter is withdrawn, leaving the stent in place. During placement, the stent can optionally be covered by a removable sheath or other means to protect both the stent and the vessels.

For self-expanding stents, the following procedure can be applicable. In order to deliver a stent to the site of a stenotic lesion (implantation site), the external diameter of the stent is reduced so that the stent can easily traverse the blood vessels leading to the implantation site. The stent is disposed within the reduced diameter portion of the vessel. Thus, the stent is reduced by, for example, elongating the stent, allowing for a corresponding reduction in diameter, and maintained in such a reduced diameter or collapsed configuration during the delivery process. Once at the implantation site, the forces tending to reduce the diameter of the stent are released whereby the stent can support and/or dilate the stenotic portion of the vessel.

In some aspects, the stent can be delivered to an implantation site by placing the reduced diameter stent within a delivery sheath that is in turn fed through a guide catheter through the vasculature to the implantation site. The stent carrying sheath is then advanced from the distal end of the guide catheter over a guide wire into the targeted vessel and to the implantation site (site of a stenotic lesion).

A second sheath can be provided proximally of the collapsed stent and used to facilitate removal of the stent from the outer sheath. For example, once the sheath has been disposed at the implantation site of a vessel, the inner, proximal sheath is held in place while the outer sheath is retracted or pulled proximally with respect to the stent. Removal of the outer sheath removes the forces that retain the stent in its collapsed configuration and thus allow the stent to self-expand within the stenotic portion of the vessel to support and dilate the vessel walls. The inner sheath prevents the stent from moving proximally with the outer sheath. The inner and outer sheaths as well as the guide wire and guide catheter can then be removed from the vascular system. Alternatively, the inner and outer sheaths can be removed and a balloon catheter fed through the guide catheter over the guide wire and into the expanded stent. The balloon can then be inflated within the stent so as to urge the stent into firm engagement with the walls of the vessel and/or to augment the dilation of the artery effected by the stent alone.

In some aspects, the stent can be delivered to the implantation site on a balloon catheter. Such balloon catheters are well known and will not be described in more detail here.

In some aspects, the biodegradable composition includes polymers that are surface erodible and bulk erodible biodegradable materials. Surface erodible materials are materials in which bulk mass is lost primarily at the surface of the material that is in direct contact with the physiologic environment, such as body fluids. Bulk erodible materials are materials in which bulk mass is lost throughout the mass of the material; in other words, loss of bulk mass is not limited to mass loss that occurs primarily at the surface of the material in direct contact with the physiological environment.

In some aspects, the biodegradable composition is composed of only biodegradable polymers. In other words, the components of the biodegradable composition are selected to be broken down by the body over time.

Typically, current drug-eluting stents release anti-restenosis agent over a period of four (4) or more weeks. In some aspects, the inventive biodegradable compositions can provide a controlled release of bioactive agent to thereby provide a therapeutically effective dose of the bioactive agent for a sufficient time to provide the intended benefits. The controlled release includes both an initial release and subsequent sustained-release of the bioactive agent.

According to the invention, the device can optionally further include a sheath that is configured to surround and enclose the device. Generally, the sheath is composed of crosslinked polymer to maintain some structural integrity during biodegradation. Optionally, the sheath can include bioactive agent. When included, one or more bioactive agents within the sheath can be the same or different from the bioactive agent(s) included in the body of the device. Optionally, the sheath can be configured to encourage cell growth (for example, by inclusion of bioactive agent and/or topography).

It will be readily appreciated that the sheath is an optional component. In some embodiments, the device can be incorporated into tissues at the implantation site as the device degrades. For example, tissue can grow into the device during use, with tissue gradually and eventually associated with the device material in a nonreleasable manner, such that even portions of the device that separate from the body do not leave the implantation site. The inventive articles, methods and systems contemplate partial or complete tissue ingrowth. Typically, at least some degree of tissue ingrowth occurs at the implantation site (but again, tissue ingrowth is not required according to the invention). The sheath can be included when it is desirable to contain pieces of the biodegradable polymer as the polymer degrades. In some embodiments, the sheath is configured to allow only pieces of polymer material of a selected size to pass through, and thereby enter the body. These configurations can be particularly desirable, for example, in vascular applications, where it can be significant to reduce the occurrence of undesirably large particles entering the blood stream, thereby posing risk of emboli. In some aspects, the sheath can function to retain the portions of the biodegradable device after the applicable portions have degraded. In other words, when the stent is fabricated from biodegradable material, the sheath can function to retain portions of the device once the overall integrity of the stent has been reduced to non-functional (for example, non-structural) pieces of polymeric material. Likewise, when the biodegradable material forms a coating on a stent, the sheath can function to retain portions of the coating that have separated from the stent during the degradation process. Such portions/pieces of the polymeric material can be retained by the sheath unless or until such portions/pieces are reduced to a size that does not pose a risk (for example, a risk of causing emboli) to the patient.

The sheath can be coupled with the stent (for example, utilizing photoreactive groups or thermochemically reactive groups, as described herein). Alternatively, the sheath can be fabricated to encase the stent without being coupled with the stent. According to this latter embodiment, the sheath can form a cladding around the stent and remain associated with the stent by virtue of encasing the stent (as opposed to being chemically coupled to the stent). Put another way, the sheath need not be chemically bonded to the stent according to the invention. According to some aspects of the invention, coupling of the sheath to the polymeric material (PEGT/PBT) forming the surface of the stent does not significantly adversely affect biodegradability of the PEGT/PBT polymeric material.

The sheath can be fabricated from a number of materials. In one embodiment, for example, the sheath is fabricated from a matrix of polymeric material such as those described in U.S. patent application Publication No. 2003/0129130 Al (Guire et al., “Particle Immobilized Coatings and Uses Thereof,” Published Jul. 10, 2003).

According to this embodiment, the matrix can be composed of a variety of polymeric material. As used herein, “polymer” and “polymeric material” refer to polymers, copolymers, and combinations and/or blends thereof that can be used to form the matrix. The polymeric material utilized for formation of the matrix can also be referred to as “matrix-forming material,” or “matrix-forming polymeric material.” In some cases the polymeric material is referred to as a “soluble polymer.” Illustrative materials for the matrix of polymeric material include, but are not limited to, synthetic hydrophilic polymers that include polyacrylamide, polymethacrylamide, polyvinylpyrrolidone (PVP), polyacrylic acid, polyethylene glycol, polyvinyl alcohol, poly (HEMA), and the like; synthetic hydrophobic polymers such as polystyrene, polymethylmethacrylate (PMMA), polybutyl methacrylate (PBMA), polyurethanes, and the like; copolymers thereof, or any combination of polymers and copolymers. Natural polymers can also be used and include polysaccharides, for example, polydextrans, glycosaminoglycans, for example hyaluronic acid, and polypeptides, for example, soluble proteins such as albumin and avidin, and combinations of these natural polymers. Combinations of natural and synthetic polymers can also be used.

In one embodiment, the polymers and copolymers as described are derivatized with a reactive group, for example, a latent reactive group such as a thermochemically reactive group or a photoreactive group. The reactive groups can be present at the terminal portions (ends) of the polymeric strand or can be present along the length of the polymer. In one embodiment, the reactive groups are located randomly along the length of the polymer.

The choice of reactive group (for example, the particular type of photoreactive group, or the choice of thermochemically reactive group over photoreactive groups) can depend upon a number of factors. For example, when the invention includes bioactive agent, it can be desirable to utilize thermochemically reactive groups as the reactive group, since many bioactive agents can be susceptible to inactivation during irradiation by light in certain wavelength ranges. Alternatively, inactivation of the bioactive agent can be reduced or avoided by choosing photoreactive groups that are activated by light outside the wavelength range that can affect the bioactive agent. According to these aspects of the invention, inactivation of the bioactive agent means degradation of the bioactive agent sufficient to reduce or eliminate the therapeutic and/or prophylactic effectiveness of the bioactive agent.

In some embodiments, polymer crosslinking compounds, for example photoreactive or thermochemically activated polymer crosslinkers, can be added to the polymeric material and can be treated to form the matrix. As used herein, “polymer crosslinking compound” refers to a compound that can be used to crosslink polymers, copolymers, or combinations thereof, together. The polymer crosslinking compound can include one or more reactive groups, and these groups can be used to crosslink the polymer and/or attach the polymer to the surface of the stent. One example of a useful polymer crosslinking compound is bisacrylamide.

In forming the polymeric matrix, the polymer and a polymer crosslinking compound can be applied to the stent and then treated to crosslink the polymers. The polymer can be crosslinked, for example, by activation of reactive groups provided by the polymer. Addition of polymer crosslinking compounds can serve to make the matrix of polymeric material more durable to use conditions and also can create matrices with controllable pore sizes. The applicability of pore size in the sheath (polymeric matrix material) is described in more detail elsewhere herein.

In some embodiments, the reactive groups provided on the polymer can be photoreactive groups, and the photoreactive polymer can be crosslinked by irradiation. The reactive groups can also serve to bind the polymer to the surface of the stent upon activation of the photoreactive groups.

According to the invention, a “photoreactive polymer” can include one or more “photoreactive groups.” A “photoreactive group” includes one or more reactive moieties that respond to a specific applied external energy source, such as radiation, to undergo active species generation, for example, active species such as nitrene, carbenes and excited ketone states, with resultant covalent bonding to adjacent targeted chemical structure. Examples of such photoreactive groups are described in U.S. Pat. No. 5,002,582 (Guire et al., commonly owned by the assignee of the present invention). Photoreactive groups can be chosen to be responsive to various portions of the electromagnetic spectrum, typically ultraviolet, visible or infrared portions of the spectrum. “Irradiation” refers to the application of electromagnetic radiation to a surface.

Photoreactive aryl ketones are preferred photoreactive groups on the photoreactive polymer, and can be, for example, acetophenone, benzophenone, anthraquinone, anthrone, quinone, and anthrone-like heterocycles (heterocyclic analogs of anthrone such as those having N, O, or S in the 10-position), or their substituted (ring substituted) derivatives. Examples of preferred aryl ketones include heterocyclic derivatives of anthrone, including acridone, xanthone and thioxanthone, and their ring substituted derivatives. Particularly preferred are thioxanthone, and its derivatives, having excitation wavelengths greater than about 360 nm.

The azides are also a suitable class of photoreactive groups on the photoreactive polymer and include arylazides (C₆R₅N₃) such as phenyl azide and particularly 4-fluoro-3-nitrophenyl azide, acyl azides (—CO—N₃) such as ethyl azidoformate, phenyl azidoformate, sulfonyl azides (—SO₂—N₃) such as benzensulfonyl azide, and phosphoryl azides (RO)₂PON₃ such as diphenyl phosphoryl azide and diethyl phosphoryl azide.

Diazo compounds constitute another suitable class of photoreactive groups on the photoreactive polymers and include diazoalkanes (—CHN₂) such as diazomethane and diphenyldiazomethane, diazoketones (—CO—CHN₂) such as diazoacetophenone and 1-trifluoromethyl-1-diazo-2-pentanone, diazoacetates (—O—CO—CHN₂) such as t-butyl diazoacetate and phenyl diazoacetate, and beta-keto-alpha-diazoacetates (—CO—CN₂—CO—O—) such as 3-trifluoromethyl-3-phenyldiazirine, and ketenes (—CH═C═O) such as ketene and diphenylketene.

Exemplary photoreactive groups are shown as follows.

TABLE 1

| Photoreactive Group | Bond Formed |
|---|---|
| aryl azides | amine |
| acyl azides | amide |
| azidoformates | carbamate |
| sulfonyl azides | sulfonamide |
| phosphoryl azides | phosphoramide |
| diazoalkanes | new C—C bond |
| diazoketones | new C—C bond and ketone |
| diazoacetates | new C—C bond and ester |
| beta-keto-alpha- | new C—C bond and beta-ketoester |
| diazoacetates | |
| aliphatic azo | new C—C bond |
| diazirines | new C—C bond |
| ketenes | new C—C bond |
| photoactivated ketones | new C—C bond and alcohol |

The photoreactive polymer can, in some embodiments, comprise a photoreactive copolymer. The polymer or copolymer can have, for example, a polyacrylamide backbone or be a polyethylene oxide-based polymer or copolymer. One example of a photoreactive polymer comprises a copolymer of vinylpyrrolidone and N-[3-(4-Benzoylbenzamido)propyl]methacrylamide (BBA-APMA); another example is a copolymer of acrylamide and BBA-APMA.

The photoreactive groups of the photoreactive polymer can allow the formation of a covalent bond between the substrate and the photoreactive polymer thereby binding the polymer to the surface of the substrate. The photoreactive groups of the photoreactive polymer can also serve to crosslink polymeric strands together, allowing the formation of a network of covalently crosslinked polymeric strands. When microparticles are included in the polymeric material (as described elsewhere herein), the crosslinked structure can serve as the matrix in which the microparticles can be entrapped. In some embodiments, a non-photoreactive crosslinking agent can be used to promote the formation of crosslinked polymeric strands. The use of a polymer crosslinking agent can depend, for example, on the location and number of photoreactive groups that are present on the polymeric strand. A polymer crosslinking agent can be added that can be a target for the photoreactive groups, that can initiate further polymerization of the polymers, or that can be thermochemically activated crosslinker, for example a DSS (N,N-disuccinimidyl suberate) crosslinker. The crosslinking agents can further solidify the matrix by bonding to other parts of the polymer.

According to some aspects of the invention, the pore size of the polymeric material comprising the sheath can be selected depending upon the application of the inventive implantable device. The pore size should be selected to provide permeability of the sheath to elements required for degradability of the polymeric material of the stent. For example, in embodiments where the stent is fabricated or coated with PEGT/PBT polymer, the sheath should include pores sufficient to allow passage of water through the sheath, thereby permitting hydrolysis of the PEGT/PBT polymeric material. In some embodiments, the pore size can be selected to allow release of elements to the implantation site. In some embodiments, when bioactive agent delivery is also accomplished by the inventive device, the sheath should include pores of sufficient size to allow release of the bioactive agent included in the stent.

In still further embodiments, the sheath can include microparticles that can contain bioactive agent. In some aspects, the pore size is sufficient to provide desired features, such as containment of microparticles within the sheath, containment of degradation products, and the like. In other words, the sheath can function to retain microparticles and/or retain degradation particles of microparticles and/or any biodegradable material utilized in association with the device and located within the sheath. For example, the pore size can be selected to permit entrapment of the microparticles within the polymeric matrix material comprising the sheath. For example, if entrapping microparticles with an average diameter of 2.5 μm, it can be useful to have a pore size in the range of 50 nm to 2.5 μm, or in the range of 100 nm to 1 μm. In any event, one of skill in the art can select a pore size by determining the maximum size of particle (regardless of source of the particle, and thereby including degradation products as well as microparticles themselves) that can be released from the degradable device. In some embodiments, particularly vascular applications of the device, such maximum size can be related to the size of particles believed to be a risk for causing embolism.

In one embodiment, the matrix of polymeric material is permeable to various compounds, the compounds typically being smaller than the smallest microparticle immobilized in the matrix. For example, in polymeric matrices that include an insoluble polymeric material, aqueous solutions which can include proteins and other molecules smaller than proteins can diffuse through the matrix.

In one embodiment, a matrix is formed from polymeric material sufficient to entrap the microparticles of the invention and also sufficient to allow the diffusion of molecules in and out of the matrix. In this embodiment, the matrix allows the immobilization of microparticles that are at least 100 nm diameter and allows the diffusion of molecules that are 50 nm or less, or 25 nm or less, in and out of the matrix.

Generally speaking, the pore size can be selected depending upon the size of elements to diffuse through the sheath during use. Such passage can be determined by the size of the elements intended to pass through the sheath to reach the device, as well as the size of the elements intended to leave the device and reach the implantation site.

In some aspects of the invention, microparticles can be included in one or more components of the device. According to the invention, microparticles can be provided in the form of microspheres and/or fibers (also referred to herein as “fibrous elements”). The microparticles can be provided with or without bioactive agent. The microparticles can be biodegradable, but this is not required. Microparticles can be included in association with the device to provide one or more features, such as, for example, enhanced imaging of the device, bioactive agent delivery, and/or desirable surface topography.

In some aspects of the invention, microparticles are included in the sheath. According to these aspects, a mixture is prepared that includes microparticles and polymer material, and the mixture is disposed on the stent and treated to provide the stent with a coating of microparticles immobilized in a matrix of polymer material. In some embodiments, the microparticles are coupled to or associated with one or more functional agents. Such functional agent can be a compound or composition that provides the device with a useful property, such as a biologically, chemically, or physically useful property.

In other aspects, the polymeric material comprising the device (such as a stent) can include microparticles, either alone or in combination with microparticles in the sheath.

The inclusion of microparticles in the sheath and/or the body of the device can provide one or more desirable features to the inventive device and methods. In one aspect, inclusion of microparticles can provide a simple and efficient method for preparing surfaces having diverse properties. For example, inclusion of microparticles can be utilized to provide a surface that can have both biologically useful and detectable properties. In another aspect, the use of microparticles can provide surfaces that are capable of delivering bioactive agent that are not typically compatible in one solvent. In a further aspect, the inclusion of microparticles can provide a cell-reactive surface to the device, as will be described in more detail herein. In still another aspect, the presence of microparticles in association with the sheath and/or device body can provide a fast and accurate method for preparing surfaces having a precise amount of bioactive agent.

When microparticles are associated with the sheath, a mixture containing a polymeric material and microparticles can be directly disposed on a surface of a stent and then treated to form a polymeric matrix to immobilize the microparticles in the matrix on the surface. Alternatively, the polymeric material can be disposed on a stent and treated, and microparticles can be subsequently disposed on the treated material and immobilized on the stent.

When microparticles are associated with the device body itself, the microparticles can be included in polymeric material that forms the device body and/or polymeric material that forms a coating on the surface of the device body. Similar to the embodiment described above, a mixture containing polymeric material and microparticles can be directly disposed on a surface of a device body and then treated to form a polymeric matrix and thereby immobilize the microparticles in the matrix on the surface. Alternatively, the polymeric material can be disposed on a stent and treated, and microparticles can be subsequently disposed on the treated material and thereby immobilized on the device body. When the microparticles are incorporated in the device body itself, the polymeric material can be formed into the device body (utilizing any of the methods described herein), and the microparticles can be provided in the polymeric material during formation of the device body.

In some embodiments, the sheath includes microparticles to provide a cell-reactive surface. “Cell-reactive” refers to the ability of coated substrate to have an effect on cells, tissue, and/or other biological material that can be in contact with the coated substrate. Cells, tissue, and other biological material include eukaryotic cells, prokaryotic cells, viruses, other biological particles, and any type of biological material the cells or particles may produce, for example, extracellular material. The sheath surface can be prepared to promote or inhibit the attachment of cells to the sheath, or can be used to provoke a cellular response by passive interaction of the cell with the sheath surface. The cell-reactive surface can be provided by the surface topography of the surface coated with polymeric material and microparticles. For example, microparticles of an appropriate size can be used to either promote or inhibit the interaction of cells, as it has been shown that size of microspheres can contribute to the interaction of certain cell types (See, for example, Mescher, M. F. (1992) J. Immunol, 149:2402). Microparticles can also be coupled to various moieties that are reactive with cell surface proteins and that can induce cellular responses.

The microparticles of the invention can comprise any three-dimensional structure that can be immobilized within a polymeric matrix. In some embodiments, the microparticle can also be associated with at least one agent. In these embodiments, the agent or agents associated with the microparticle can impart a desirable property to the surface of the substrate.

According to the invention, the microparticle can be fabricated from any differentially soluble or solid material. Suitable materials include, for example, synthetic polymers such as poly(methylmethacrylate), polystyrene, polyethylene, polypropylene, polyamide, polyester, polyvinylidenedifluoride (PVDF), and the like; degradable polymers such as poly(lactide-co-glycolide) (PLGA) and chitosan (poly-(1,4)-β-D-glucosamine), and the like; glass, including controlled pore glass (CPG) and silica (nonporous glass); metals such as gold, steel, silver, aluminum, silicon, copper, ferric oxide, and the like; natural polymers including cellulose, crosslinked agarose, dextran, and collagen; magnetite, and the like. Examples of useful microparticles are described, for example, in “Microparticle Detection Guide,” from Bangs Laboratories, Fishers, Ind. Optionally, microparticles can be obtained commercially from, for example, Bangs Laboratories (Fishers, Ind.), Polysciences (Germany) Molecular Probes (Eugene, Oreg.), Duke Scientific Corporation (Palo Alto, Calif.), Seradyn Particle Technology (Indianapolis, Ind.), and Dynal Biotech (Oslo, Norway).

In some embodiments, the microparticles are not modified prior to preparation of the microparticle-containing mixture and disposing of the microparticles on the substrate. In these embodiments, the microparticle itself can provide a desirable or useful property when associated with the polymeric matrix on a substrate. For example, paramagnetic microparticles compose of, for example, iron oxide, can provide the surface of a substrate with paramagnetic properties; silica can provide the surface of a substrate with refractive properties; and metallic microparticles can provide the surface of a substrate with reflective properties. In yet another example, microparticles of a suitable size can provide a surface of a substrate that is suitable for interactions with various cell types.

When microparticles are provided in the form of microspheres, they can be provided in any suitable size, but preferably the microsphere is in the range of 5 nm to 100 μm in diameter, or in the range of 100 nm to 20 μm in diameter, or in the range of 400 nm to 20 μm in diameter.

In one embodiment, degradable microparticles can be utilized in association with the sheath. Degradable microparticles can include, for example, dextran, polylactic acid, poly(lactide-co-glycolide), polycaprolactone, polyphosphazene, polymethylidenemalonate, polyorthoesters, polyhydroxybutyrate, polyalkeneanhydrides, polypeptides, polyanhydrides, polyesters, and the like. Degradable polymers useful in the invention can be obtained from, for example, Birmingham Polymers, Inc. (Birmingham, Ala.). Degradable polymers and their synthesis have also been described in various references including Mayer, J. M., and Kapalan, D. L. (1994) Trends in Polymer Science 2:227-235; and Jagur-Grodzinski, J. (1999) Reactive and Functional Polymers: Biomedical Application of Functional Polymers, 39:99-138.

In some cases, the degradable microparticles can be a mixture of a degradable material and a plastic. The degradable material is also preferably nontoxic, although in some cases the microparticles can include an agent that is useful for the selective prevention of prokaryotic or eukaryotic cell growth, or elimination of cells, such as chemotherapeutic agents or antimicrobials. Degradable microparticles can include bioactive agents that can be released from the sheath upon degradation of the microparticle.

In one embodiment, the degradable microparticles can contain a bioactive agent. Degradable microparticles can be prepared incorporating various bioactive agents by established techniques, for example, the solvent evaporation technique (See, for example, Wiehert, B. and Rohdewald, P., J Microencapsul. (1993) 10:195). The bioactive agent can be released from the microparticle, which is immobilized in the polymeric matrix on a stent, upon degradation of the microparticle in vivo. Microparticles having bioactive agent can be formulated to release a desired amount of the bioactive agent over a predetermined period of time. It is understood that factors affecting the release of the bioactive agent and the amount released can be altered by the size of the microparticle, the amount of agent incorporated into the microparticle, the type of degradable material used in fabricating the microparticle, the amount of microparticles immobilized per unit area on the substrate, and the like. The bioactive agent or agents associated with the microparticle can be the same or different from any bioactive agent or agents associated with the polymeric material utilized to fabricate the stent and/or coating on a stent.

In one embodiment, the invention advantageously allows for preparation of surfaces having two, or more than two, different functional agents, wherein the functional agents are mutually incompatible in a particular environment, for example, as hydrophobic and hydrophilic bioactive agents (drugs) are incompatible in either a polar or non-polar solvent. Different functional agents may also demonstrate incompatibility based on protic/aprotic solvents or ionic/non-ionic solvents. For example, the invention allows for the preparation of one set of degradable microparticles containing a hydrophobic drug and the preparation of another set of degradable microparticles containing a hydrophilic drug; the mixing of the two different sets of microparticles into a polymeric material used to form the matrix; and the disposing of the mixture on the surface of a substrate. Both hydrophobic and hydrophilic drugs can be released from the surface of the coated device at the same time, or the composition of the degradable microparticles or polymeric matrix can be altered so that one drug is released at a different rate or time than the other one.

As mentioned herein, the device body can be fabricated to include the bioactive agent in the body itself, either in addition to, or as a substitute for, bioactive agent included on the surface of the device. Optionally, a sheath can be provided as well. Use of microparticles in the device body itself can provide the ability to prepare the device to include otherwise incompatible functional agents, as described above.

In some cases it can be advantageous to prepare degradable microparticles having a composition that is more suitable for either hydrophobic or hydrophilic drugs. For example, useful degradable polymers or degradable copolymers for hydrophobic drugs have a high lactide or high caprolactone content; whereas useful degradable polymers or degradable copolymers for hydrophilic drugs have a high glycolide content.

Traditional coating procedures directed at disposing at least two different types of functional agents have often required that the functional agents be put down onto a substrate separately. In one such example, the coating procedure can involve solubilizing a hydrophobic drug in a non-polar solvent, coating the surface of the substrate with the non-polar mixture, drying the non-polar mixture, solubilizing the hydrophilic drug in a polar solvent, coating the layer of the dried non-polar mixture with the polar mixture, and then drying the polar mixture. This process can be inefficient and can also result in undesirable surface properties (for example, the layering of the drugs can cause one drug to be released before the other one is released). According to the invention, the method of preparing a sheath having two, or more than two, different functional agents, in particular when the two different functional agents are released from the sheath polymeric material, is a significant improvement over traditional methods of coating substrates and delivering functional agents from the surface of the substrates.

Other types of non-degradable microparticles can also be useful for the release of a functional agent from the sheath. Such non-degradable microparticles include pores and can be silica microparticles, for example. Porous non-degradable microparticles can also be used for incorporation of an agent, such as a bioactive agent. Microparticles having particular pore sizes can be chosen based on the type and size of the agent to be incorporated into the pores. Generally, the microparticle having pores can be soaked in a solution containing the desired agent wherein the agent diffuses into the pores of the microparticle. Substrates can be prepared having a coating of these microspheres in a polymeric matrix. Upon placing the coated substrate in fluid-containing environment, for example in a patient, the agent can be released from the microspheres and be delivered to the patient.

The type of polymer, as well as the concentration of the polymer and the extent of polymer crosslinking in the polymeric matrix, can have an affect on the delivery of the bioactive agent from the sheath. For example, polymeric matrix material having charged portions can either decrease or increase the rate of release of a charged bioactive agent from the sheath, depending on whether there are attractive or repulsive forces between the two. Similarly, hydrophilic and hydrophobic polymeric matrix material can also have an affect on the rate of release of hydrophilic and hydrophobic bioactive agents, in particular hydrophilic and hydrophobic drugs. In polymeric matrices having a high concentration of polymer or in matrices wherein the polymer is highly crosslinked, the rate of delivery of the drug can be decreased.

Microparticles can also have an outer coating to control the availability of the agent or agents that are associated with the microparticle. For example, microparticles can include an outer coating of poly(ethylene glycol) (PEG) which can provide sustained or controlled availability of the functional agent that is associated with the microparticle. Another useful outer coating can include, for example, a silane or polysiloxane coating.

In some applications, swellable microparticles can be employed for incorporation of the functional agent. Such swellable microparticles are typically composed of polystyrene or copolymers of polystyrene, and they are typically swellable in an organic solvent. Microparticles can be soaked in organic solvents containing the functional agent to allow incorporation of the agent into the microparticle. The solvent swells the polymeric microparticles and allows the functional agent to penetrate into the microparticles' cores. Excess solvent is then removed, for example, by vacuum filtration, thereby entrapping the functional agent in the hydrophobic interior regions of the microparticles. In one such embodiment, poly(methylstyrene)-divinyl benzene microparticles are rinsed in dimethylformamide. A solution containing the functional agent in dimethylformamide is then added to the microparticles, and the microparticles and solution are incubated with agitation overnight. Excess functional agent is removed from the suspension by vacuum filtration using membrane filters, such as those provided by Millipore Company (Bedford, Mass.). The filtered microparticles are then sonicated and washed by centrifugation in distilled water containing 0.01% Tween 20 to remove residual functional agent on the outside of the microparticles.

In some embodiments it is preferable that the swellable microparticle is impregnated with a functional agent that is detectable using common imaging techniques, for example a paramagnetic material, such as nanoparticular iron oxide, Gd, or Mn, a radioisotope, and non-toxic radio-opaque markers (for example, cage barium sulfate and bismuth trioxide). This can be useful for detection of medical devices that are implanted in the body (that are emplaced at the treatment site) or that travel through a portion of the body (that is, during implantation of the device). Such coated medical devices can be detected by paramagnetic resonance imaging, ultrasonic imaging, or other suitable detection techniques. In another example, microparticles that contain a vapor phase chemical can be used for ultrasonic imaging. Useful vapor phase chemicals include perfluorohydrocarbons, such as perfluoropentane and perfluorohexane, which are described in U.S. Pat. No. 5,558,854 (Issued 24 Sep., 1996); other vapor phase chemicals useful for ultrasonic imaging can be found in U.S. Pat. No. 6,261,537 (Issued 17 Jul., 2001).

The microparticles of the invention can possess one or more desirable properties, such as ease of handling, dimensional stability, optical properties, sufficient size and porosity to adequately provide the desired amount of agent or agents to a sheath and/or device body, and the like. The microparticles can be chosen to provide additional desired attributes, such as a satisfactory density, for example, a density greater then water or other solvent used in application of the microparticles to the substrate.

Optionally, the microspheres can include a “coupler” that can allow the coupling of a functional agent to the microparticle. As used herein, the terms “coupler,” “coupling compound,” and “coupling moiety” refer to any sort of entity that allows a functional agent to be attached to the microparticle. The coupler can have one member or more than one member. For example, the coupler can be a small molecule, or can be a binding pair that consists of more than one larger molecule, for example a pair of interacting proteins.

The microparticles can be prepared to include a coupler having reactive groups. The coupler having reactive groups can be used for coupling one or more functional agents to the microparticle, for example, bioactive agents or functional agents conferring optical properties. In other embodiments, reactive groups provided on the microparticle can be used for coupling the microparticle to the polymeric material or for coupling the microparticle to the surface of the substrate, or any combination of the above. Suitable reactive groups can be chosen according to the nature of the functional agent that is to be coupled to the microparticle. Examples of suitable reactive groups include, but are not limited to, carboxylic acids, sulfonic acids, phosphoric acids, phosphonic acids, aldehyde groups, amine groups, thiol groups, thiol-reactive groups, epoxide groups, and the like. For example, carboxylate-modified microparticles can be used for covalent coupling of proteins and other amine-containing molecules using water-soluble carbodiimide reagents. Aldehyde-modified microparticles can be used to couple the microparticles to proteins and other amines under mild conditions. Amine-modified microparticles can be used to couple the microparticle to a variety of amine-reactive moieties, such as succinimidyl esters and isothiocyanates of haptens and drugs, or carboxylic acids of proteins. In another application, sulfate-modified microparticles can be used for passive absorption of a protein such as bovine serum albumin (BSA), IgG, avidin, streptavidin, and the like.

In another embodiment, the reactive groups can include such binding groups as biotin, avidin, streptavidin, protein A, and the like. These and other modified microparticles are commercially available from a number of commercial sources, including Molecular Probes, Inc. (Eugene, Oreg.).

Another method for coupling moieties of the invention is through a combination of chemical and affinity interactions, herein referred to as “chemi-affinity” interactions, as described by Chumura et al. (2001) Proc. Natl. Acad. Sci., 98:8480. Binding pairs can be engineered that have high binding specificity and a negligible dissociation constant by functionalizing each member of the binding pair, near the affinity binding sites of the pair, with groups that will react to form a covalent bond. For example, the constituents of each functionalized member can react, for example by Michael addition or nucleophilic substitution, to form a covalent bond, for example a thioether bond.

The surface of the microparticle can also be coated with crosslinking compounds. Various functional agents can be coupled to the microparticle via crosslinking agents. Commercially available crosslinking agents obtained from, for example, Pierce Chemical Company (Rockford, Ill.) can be used to couple the microparticles to functional agents via, for example, amine groups, provided on the surface of the microparticles. Useful crosslinking compounds include homobifunctional and heterobifunctional crosslinkers. Two examples of crosslinking compounds that can be used on microparticles presenting, for example, amine groups, are di-succinimidyl suberate and 1,4-bis-maleimidobutane.

In some embodiments, the microparticles are associated with a functional agent. As used herein, a “functional agent” refers to a compound that can be coupled to, or associated with, the microparticles to provide the surface of the coated substrate with a property that is conferred by that compound. Useful functional agents include bioactive agents, compounds with detectable properties, such as paramagnetic compounds, and compounds with optical properties. The microparticles of the invention can be coupled to, or associated with, any physiologically active substance that produces a local or systemic effect. For ease of discussion, reference will repeatedly be made to a “functional agent.” While reference will be made to a “functional agent,” it will be understood that the invention can provide any number of functional agents to a treatment site. Thus, reference to the singular form of “functional agent” is intended to encompass the plural form as well.

The quantity of functional agents associated with each individual microparticle can be adjusted by the user to achieve the desired effect. Factors that can influence this can be, for example, the amount of anti-coagulant activity. The density of functional agents coupled to, or associated with, the microparticles can vary and can depend upon, for example, the dose of a particular bioactive agent intended to be provided on the sheath. Bioactive agents can be provided by the microparticles in a range suitable for the application. In another example, protein molecules can be provided by microparticles. For example, the amount of protein molecules present can be in the range of 1-250,000 molecules per 1 μm diameter microparticle. However, depending on microparticle source and preparation the amount of agent coupled to, or associated with, the microsphere can vary.

The quantity and organization of the microparticles themselves within or on a sheath can also impart desirable properties to the stent, for example, in imagining the device within the patient's body. For paramagnetic resonance or ultrasonic imaging applications, the number of microparticles associated with a device can be directly correlated with the imaging signal strength. To increase imaging signal strength, a high density of microparticles can be immobilized in a localized area on the device. Alternately, the density of microparticles over the device can vary, thereby allowing different regions of the device to be imaged distinctly. This can be accomplished by coating the different regions of the device with two or more different coating slurries with differing concentrations of microparticles.

Coupling the functional agent to, or associating the functional agent with the microparticle prior to disposing the microparticle on the sheath can provide benefits. It is understood that the functional agent can be provided within or on the surface of microparticles. For example, as compared to directly coupling an agent to a substrate, a higher density of agent per surface area of substrate can be achieved by first loading the functional agent on or in the microparticle. Also, coupling of an agent to the microparticle in solution is generally more efficient than the direct coupling of a functional agent to a substrate, resulting in a lower loss of functional agent during the coupling procedure. Additionally, coupling of a functional agent to a microparticle in solution generally allows for more variability during the coupling process. For example, coupling procedures that require agitation of the coupling solution, such as stirring, can readily be achieved using microparticles in the stirred solution. Additionally, determination of the amount of functional agent coupled per microparticle can readily be achieved by performing, for example, immunofluorescence flow cytometry or a protein assay, such as a BCA assay, on a portion of the microparticles following coupling to the functional agent. Once the microparticles have been coupled with the desired amount and type of functional agent, these functional agent-coupled microparticles can then be included in a mixture containing a suitable polymeric material or can be disposed on a substrate that has been coated with a polymeric material.

In some embodiments, the functional agent can be modified prior to coupling with the microparticle. In other words, a portion of the coupler can be attached to the functional agent prior to the functional agent being coupled to the microparticle. For example, the functional agent can be derivatized with one member of a binding pair, and the microparticles derivatized with the other member of the binding pair. Suitable binding pairs include avidin:biotin, streptavidin:biotin, antibody:hapten, for example anti-digoxigenin Ab:digoxigenin or anti-trinitrophenyl Ab: trinitrophenyl. For example, the functional agent can be biotinylated by, for example, cross-linking the biotin to the functional agent using methods known in the art. The biotinylated agent or agents can then be coupled with streptavidin provided on the surface of the microparticles. Members of the binding pair can be functionalized to provide chemi-affinity interactions as indicated elsewhere herein.

As described herein, the microparticles can be immobilized in the polymeric matrix forming the sheath by entrapment of the microparticles. In another embodiment, immobilization of the microparticles can be performed by chemical bonding of the microparticle to the matrix and the matrix to the substrate. A variety of bonds can be formed between the microparticles and the matrix material, and the matrix material and the substrate. These bonds include, for example, ionic, covalent, coordinative, hydrogen and Van der Waals bonds. For example, it can be desirable to maintain the microparticles within the sheath (as opposed to releasing the particles and/or allowing the microparticles to degrade over time within the patient). This can occur, for example, when the microparticles are utilized for imaging the device within the patient, or when heparin is provided on the surface of the sheath and it is desired to maintain the heparinized surface on the device while the device is in the patient.

In one embodiment, slurries including polymeric material and microparticles, which can be coupled to, or associated with, a functional agent, are dip-coated onto the surface of the stent to form a coated surface (sheath). In another embodiment the polymeric material is dip-coated to form a coated surface (sheath). Alternatively, the polymeric material can be applied by jet printing to the surface of the substrate through utilization of a piezoelectric pump. Printing techniques can allow the application of a relatively small amount of the mixture at precise locations on the surface of the substrate. In another embodiment, the polymeric material is disposed on the substrate and treated; the microparticles are then placed and immobilized on the substrate via the treated material.

In some embodiments, the thickness of the matrix of polymeric material forming the sheath is greater than the diameter of the largest microparticle being associated with the sheath. However, providing a matrix having a thickness greater than the diameter of the largest microparticle is not required, and microparticles can be immobilized without completely entrapping the microparticle within the matrix material. In some applications, the stent can be subject to more than one step of coating with a mixture of polymeric material and microparticles and treating, thereby allowing the formation of a sheath composed of multiple layers.

In some embodiments, microparticles are provided in the form of fibers. Optionally, the fibrous element can comprise a non-biodegradable element of the overall device. Alternatively, the fibrous element can comprise a biodegradable element of the device. In still further embodiments, the fibrous element can be selected and formulated to degrade at a different rate than other elements of the overall medical device. Generally, fibrous elements (whether biodegradable or not) can be desired, for example, to provide additional structural support to the device, and/or to provide a cell-reactive surface to the device. The choice of biodegradable or non-degradable material to fabricate the fibrous element can depend upon the application of the device, and whether the user desires to maintain the fibrous elements within the patient's body after other portions of the device degrade. The fibrous elements can be embedded within the degradable polymeric material, provided on a surface of the degradable polymeric material, or embedded within and provided on a surface of the degradable polymeric material.

In one such embodiment, non-biodegradable fibers are included in the degradable polymeric material used to make a stent. According to this embodiment, the polymeric material comprising the stent will degrade over time, leaving the non-biodegradable fibers at the implantation site. The fibers can provide additional radial force to reduce occurrence of collapse/restenosis at the implantation site, during residence of the medical device, as well as after the degradable portion of the device has broken down in the body.

Fibrous elements can be included within the degradable polymeric material in a number of ways. In one embodiment, fibers are added to a mixture of dimethylterephthalate, butanediol (in excess), polyethylene glycol, an antioxidant, and catalyst. The reaction mixture is then subject to a synthesis procedure described elsewhere herein (the particular synthesis procedure will depend, of course, upon the polymeric material; for example, when the polymeric material comprises PEGT/PBT, the synthesis generally includes steps of transesterification, distillation of excess butanediol, and condensation of a prepolymer of butanediol terephthalate with the polyethylene glycol to form a PEGT/PBT copolymer). In an alternative embodiment, a polymer (such as a PEGT/PBT copolymer) can be formed and subsequently subjected to temperatures sufficient to “melt” the polymer. According to this embodiment, the polymer will achieve a temperature sufficient to allow fibers to be mixed within the polymer melt, but not sufficient to alter the properties of the polymer for its intended use. After the fibers are mixed with the polymer melt, the melt can be permitted to form a solid polymer material through evaporation of solvent or through cooling of the melt.

In yet a further embodiment, the fibers can be combined with a reactive polymer, followed by polymerization to form a polymeric matrix that includes the fibrous material. For example, polymeric matrix structures can be formulated by mixing selected monomeric components with polymerization facilitating compounds, such as one or more initiators and/or activators. One illustrative polymeric matrix has been formulated by I. Chung et al. (European Polymer Journal 39:1817-1822 (2003)). Chung et al. formulated network structures by thoroughly mixing selected oligomers with a photoinitiator and an activator. More specifically, polycaprolactone trimethyacrylate (PCLTMA) and di(propylene fumarate)-dimethacrylate (DPFDMA) were mixed with DL-camphoroquinone (CQ, 0.7 weight %, a photoinitiator) and 2-(dimethylamino)ethyl methacrylate (DMAEM, 1.4 weight %, an activator). The mixture was then exposed to blue light source for ten minutes at room temperature. The cured specimens were then removed from molds and conditions in PBS solution. By modifying the formulation of the polymeric matrices, such features as degradation rates, strength, viscosity were controllable. Thus, such matrices could be utilized in the inventive methods and devices as well. Fibrous elements can be combined with the monomeric components and polymerization facilitating compounds and polymerized to form polymeric network structures that include fibrous elements. Other reactive polymers are known and can be readily adapted for use with the inventive concepts described herein. These coated fibers can then be mixed with the degradable copolymers described herein.

Preparation methods for fibrous polymer materials are described, for example, in U.S. Pat. No. 6,685,957 (Bezemer et al., “Preparation of Fibrous Polymer Implant Containing Bioactive Agents Using Wet Spinning Technique”) and U.S. patent Publication No. US 2004/0086544 (Bezemer et al., “Polymers with Bioactive Agents”). According to these particular embodiments, a wet spinning technique is utilized to provide polymer loaded with one or more bioactive agents. Preparation of one such copolymer will be explained by way of example for a PEGT/PBT copolymer. Utilizing the teaching herein, the skilled artisan will be able to prepare any number of copolymers that include bioactive agent.

A PEGT/PBT copolymer can be synthesized as described above (transesterification, followed by distillation, and condensation). The bioactive agent to be loaded into the polymer can be chosen from any suitable bioactive agent. Some exemplary bioactive agents are mentioned herein. Generally, the bioactive agent-loaded polymer is formed by preparing an aqueous solution of the bioactive agent, and adding the bioactive agent solution to a solution of amphiphilic block copolymer containing hydrophobic blocks dissolved in a first solvent that is immiscible with water to form an emulsion. The emulsion is injected through a nozzle into a second solvent that is miscible with the first solvent and in which the copolymer is essentially insoluble. The result after injection is a solid copolymer fiber loaded with the bioactive agent. The fiber can then be shaped into an implant, if desired. Typically, for preparation of the water-in-oil emulsion according to these embodiments, it is desired that a hydrophobic bioactive agent dissolves at least slightly in water, preferably at least to such an extent that the resultant loaded polymer comprises an amount of the bioactive agent sufficient to achieve a desired effect in vivo. Optionally, a surfactant can be added to the aqueous solution of the bioactive agent in order to allow a minimal desired amount of the bioactive agent. Examples of such surfactants are well known to the skilled artisan and can be used in amounts that can easily be optimized by the artisan. Specific examples of suitable surfactants include, but are not limited to, poly(vinyl) alcohol, Span 80, Tween, and Pluronic.

According to these embodiments of the invention, two solvents are chosen to complement each other's action in the synthesis process. The first solvent is chosen to be immiscible with water. In addition, the polymer that is to be loaded with bioactive agent should be soluble in the first solvent. The second solvent is chosen such that the polymer is insoluble therein. Also, the first solvent is selected to be well miscible with the second solvent. Preferably, the first solvent mixes better with the second solvent than the polymer dissolves in the first solvent. This helps ensure that, upon immersion of the water-in-oil emulsion in the second solvent, the first solvent will substantially completely migrate into the second solvent. Preferably, both the first and second solvents are immiscible with water. This makes it possible to prevent contact between the bioactive agent, which is processed in an aqueous solution, with an organic solvent, which can be harmful to the bioactive agent. Depending upon the nature of the polymeric material to be loaded, the skilled person can readily select suitable solvents utilizing the teaching herein. By way of example, when the polymer is PEGT/PBT copolymer, a suitable first solvent is chloroform, and a suitable second solvent is hexane.

In a first step of the process, a solution is provided of the polymer in the first solvent. The concentration of this solution is not critical and can be determined based upon such factors as the amount of solvent sufficient to dissolve all of the polymer, and overall efficiency of the process.

A water-in-oil solution is prepared by mixing the polymer solution with an aqueous solution of the bioactive agent. Under certain circumstances, it can be desired to add conventional stabilizers to enhance the stability of the water-in-oil emulsion. Typical examples of such stabilizers include proteins such as albumin or casein, Pluronic, and Span 80. Such stabilizers are optional only.

According to these embodiments, the amount of bioactive agent in the aqueous solution can be chosen such that a desired amount of the bioactive agent is eventually incorporated into the polymer. The amount of bioactive agent incorporated in the polymer can depend upon such factors as the type of polymer and the nature of the bioactive agent. In the case of proteins and peptides, for example, at least 0.01 weight percent (based upon the weight of the loaded polymer) of the protein or peptide will be incorporated. For proteins and peptides, up to about 10 weight percent (based upon the weight of the loaded polymer) can be incorporated into the polymer. When using particularly hydrophilic bioactive agents, the agent can be incorporated in a concentration of up to 50 weight percent (based upon the weight of the loaded polymer).

The amount of water used for preparing the aqueous bioactive agent solution will be sufficiently high to enable an efficient dissolution of the bioactive agent without employing unduly harsh conditions that might adversely affect the stability and/or biological activity of the bioactive agent. The upper limit of the amount of water used can depend upon the rate at which the bioactive agent is to be released from the polymer in a final application. The use of larger amounts of water typically leads to higher release rates of the polymer. Typically, the aqueous solution of the bioactive agent will comprise about 0.001 to about 10 weight percent of bioactive agent, based upon the weight of the solution. In practice, the amount of bioactive agent in the solution will depend upon the solubility of the bioactive agent or agents chosen, and on the stability of the water-in-oil emulsion.

The obtained water-in-oil emulsion is next immersed in the second solvent by injection through a nozzle. The diameter and shape of the nozzle can be varied to obtain fibers of different diameter and shape. The injection itself will typically be driven by a pressure that transports the emulsion through the nozzle into the second solvent. For example, injection can be accomplished by use of a syringe or an extruder. The amount of the second solvent is not critical and can be selected to be at least sufficient for the emulsion to be completely immersed in it and to allow a substantially complete migration of the first solvent from the emulsion into the second solvent. The upper limit will generally be chosen on the basis of economic considerations.

Upon immersion of the emulsion into the second solvent, the first solvent will migrate from the emulsion into the second solvent due to the specific selection of the first and second solvents. In practice, it can often be observed that first exchange of the first and second solvents takes place before the first solvent will migrate into the second solvent. This results in polymer fibers provided with a porosity. P. van de Witte (“Polylactide membranes. Correlation between phase transitions and morphology,” PhD thesis, University of Twente, Enschede, 1994) describes this phenomenon and how it can be controlled to obtain a desired porosity.

As a result, the polymer, which does not dissolve in the second solvent, will solidify and thereby incorporate the bioactive agent. Finally, the solid loaded polymer can be removed from the mixture of the first and second solvents in any convention manner and can eventually be dried.

In some embodiments, the obtained fibers can be formed into a fibrous mesh by collecting the fibers in a mold, and bonding them together (for example, by use of a suitable solvent mixture). According to these embodiments, the mixture should comprise at least one solvent in which the polymer does not dissolve. Preferably, a mixture is used of the above described first and second solvents. The second solvent will typically be present in an amount exceeding that of the first solvent, in order to reduce the risk of any of the polymer dissolving in the solvent mixture. Preferably, the volumetric ratio of the first solvent to the second solvent is in the range of 1:1 to 1:3.

Other methods of synthesizing a polymer containing fibers are known and will not be discussed in detail herein.

Fibrous elements can be provided on a surface of the polymeric material in any suitable manner. For example, the fibers can be derivatized to include a coupling group sufficient to bind the fibers to the surface of the polymer material. In another exemplary embodiment, the fibers can be provided with a reagent, such as one or more of the reagents described in U.S. Pat. No. 4,979,959 (Guire), U.S. Pat. No. 5,002,582 (Guire et al.), U.S. Pat. No. 6,514,734 (Clapper et al.), U.S. Pat. No. 6,410,643 (Swanson), U.S. Pat. No. 6,689,473 (Guire et al.), U.S. Pat. No. 6,444,318 (Guire et al.).

In another aspect, fibers composed of polyethylene (PE) can be desirable for use in composite materials use in biomedical devices. PE fibers exhibit high strength, chemically stability, low density, and biocompatibility. However, use of PE fibers in composites has been limited largely by their surface properties, which can hinder adhesion. Thus, surface modification of such fibers can provide an improved composite material that includes the fibers.

In some embodiments, it can be preferable to modify the surface of the fibers (degradable or non-degradable), regardless of whether the fibers are provided on the surface and/or within the polymeric material. Most polymer blends are immiscible, and thus, the components of a polymer blend phase often separate into distinct, macroscopic domains. These macroscopic domains can be undesirable in a composite material, since they can lead to voids within the polymer composite material, as well as instability in the polymer blend as a result of nonhomogeneity of the polymer components.

In order to provide effective reinforcement, there should exist good stress transfer at the interface of the fiber and polymer material with which the fiber is associated. The stress transfer at the interface between two different phases in the solid state is determined by the degree of adhesion. Adhesion to fibers can be limited by their surface morphology, chemical inertness and/or low surface energy. Thus, strong chemical or physical bonding between the two materials can be important to achieve adhesion. The chemical bonding can be described by ionic, covalent, or metal bonds, whereas the physical bonding is represented by London dispersion forces, van der Waals forces, hydrogen-bonding, polar-polar bonds, and the like.

In some aspects of the invention, surface modification of fibers is achieved by chemically roughening the surface of the fiber to minimize the size of any surface defects. According to these aspects, surface roughening can be accomplished by either degrading the outer layer of the fiber or building it up by a grafting process. Methods to improve adhesion of fibers can include reactive plasmas, irradiation, chemical etching, and ozonolysis. These methods are discussed, for example, in Brennan, A. B., “Surface Modification of Polyethylene Fibers for Enhanced Performance in Composites,” Trends in Polymer Science, (1995), vol. 3:12-21.

In some aspects, surface modification is accomplished by plasma treatment, which involves a complex series of reactions with free radicals, cations, electrons, and the excited states created by the excitation of a gas at either a reduced pressure or ambient pressure. The effect of the plasma on the surface can be described in general terms as either polymer-forming or non-polymer-forming (also referred to as ablative) reactions. Polymer-forming reactions are induced by plasmas formed from most organic gases. The polymers formed by these reactions typically have reactive functional groups that enhance the formation of both chemical and physical bonds with adherents. The non-polymer-forming plasmas include those from oxygen, nitrogen, hydrogen, argon, and ammonia. The action of these plasmas involves abstraction of protons and creation of unstable radicals that, upon exposure to oxygen, convert to functional groups such as alcohols, aldehydes, ketones, and carboxylic acids. The ablative process involves removal of the outer portion (typically 5 to 50 nm) of the fiber.

Surface modification can also be accomplished by ionizing radiation from a gamma source such as ⁶⁰Co. In the presence of reactive organic monomers, ionizing radiation can create polymeric grafts on the surface of the fiber. Gamma radiation penetrates into the bulk of the fiber material and produces cations, cation radicals, free radicals, and other reactive intermediates. One illustrative example will be described. Poly(cyclohexyl methacrylate) (PCHMA), poly(N-vinylpyrrolidone (PVP) and poly(n-butyl acrylate) (PBA) can be grafted onto the surface of fibers using ⁶⁰Co gamma radiation. Typically, PE will undergo crosslinking and chain-scission reactions when exposed to high doses of gamma radiation; thus, low dosages and dose rates can be advantageous in some applications.

Surface modification of fibers by irradiation with an electron beam is another method that can be utilized.

In still further embodiments, wet chemical methods can be utilized to provide surface modification of the fiber. In contrast to the methods described above, these methods are chemical processes performed in the absence of any external radiation. Wet chemical methods typically involve strong oxidizing agents. For example, PE fibers can be coated by mixing in a solution of poly(hydroxyethyl methacrylate) (PHEMA) and dimethylformamide. The fibers can be allowed to swell in benzoyl peroxide (BPO) at 50° C. Each fiber can then be incorporated into a selected polymer mixture (including any of the polymer materials described herein) that is subsequently molded and reacted to form a composite.

Thus, to enhance the structural integrity and mechanical properties of a polymeric material associated with fibers, copolymer “compatibilizers” can be added to the polymer mixture. In some embodiments, compatibilizers effectively act as high molecular weight surfactants, in that they can localize at the interface between the immiscible polymers, interlink the phase-separated regions of the polymer blend, lower the interfacial tension, and disperse the incompatible polymers into smaller domains. Consequently, the degree of adhesion between the phase-separated regions and the mechanical properties of the material can be significantly enhanced.

One illustrative example of suitable compatibilizers includes graft copolymers. Graft copolymers contain a backbone and side chains that emanate from the backbone. The side chains of the graft copolymer intertwine across the polymer-polymer interface and effectively bind the two phase-separated regions. Gersappe, D. et al. ((1994) Science 265:1072-1074) describe suitable graft copolymers for use as compatibilizers, as well as methods to determine suitable graft copolymers for such use. For example, a four-component blend composed of two immiscible, phase-separated homopolymers, A and B, and two types of graft copolymers, AC and BD can be designed as compatibilizers. The backbones of the AC copolymers are formed entirely from A segments, whereas the side chains are formed from C units. Similarly, for the BD chains, the backbones are formed entirely of B segments, while the D segments are the side chains. Generally speaking, the A and B backbones of the compatibilizers are formed from incompatible polymers, while the C and D side chains are formed from highly compatible polymers. The high interfacial tension between the immiscible homopolymers drives the grafts to the A-B boundary. The compatibilizers can then localize at the interface, with the C and D side chains intertwining across the A-B layer. The side chains thread through and bind across the interface. Exemplary A and B homopolymers include poly(ethyl acrylate) (PEA) and poly(methyl methacrylate) (PMMA). The side chains C and D were polystyrene (PS).

Suitable fibers include fibrous materials of sufficient strength to provide the desired properties to the inventive device. For example, nanofibers are commercially available and can be utilized in accordance with the teachings herein. In embodiments where the nanofibers remain at the implantation site after degradation of the polymer, fibers with nanometer to micro diameter can be advantageous.

Optionally, the fibers can be fabricated to include one or more bioactive agents, either in addition to, or instead of, other portions of the device. Use of bioactive agent in association with the nanofibers can provide multiple bioactive agents and/or the same drug with multiple release rates to be used in connection with the same device.

The fibers can be fabricated to include bioactive agent in any suitable manner. In one embodiment, viscous polymer solutions containing bioactive agent can be forced through a small orifice into a solvent that does not dissolve the bioactive agent or the polymer material, thus creating filaments. The diameter of the filaments can be dependent upon the orifice diameter.

As discussed herein, the biodegradable polymer material can be selected and formulated to provide a desired controlled release of bioactive agent to a treatment site. In some embodiments, the configuration of the device can be manipulated to control release of the bioactive agent. For example, the surface area and/or size of the device can be manipulated to control dosage of the bioactive agent(s) provided to the implantation site. In other aspects, incorporation of the bioactive agent in microspheres, fibers, or other delivery devices, can impact release rate of the bioactive agent, as will be apparent from the discussion herein. Further, as described above, the composition of the polymeric material can itself be manipulated to affect release rate of the bioactive agent.

In designing an implantable, biodegradable medical device that can provide controlled release of a bioactive agent, it is desirable to have the capability to modulate the shape of the release curve. The time profile of the release of the bioactive agent can range from immediate release where the drug elutes all at once (much like a step function) to an extremely slow, linear (zero order) release, where the drug is evenly released over many months or years. Depending upon the drug and the condition being treated, there are a variety of release profiles that are of interest. The objective of creating medical devices fabricated of biodegradable polymers is to be able to attain the broad range of release profiles that lie between a step function and a low-slope, zero-order release.

One of the primary strategies to control the release of a bioactive agent is to limit the initial release (or “burst”) of bioactive agent. If this can be achieved, then more bioactive agent is available at later times for a more extended release duration. The utilization of biodegradable polymer materials to fabricate implantable devices as described herein is designed to limit or even eliminate the burst of bioactive agent from the device. The bioactive agent still remaining in the device after the initial burst is then released to the site of action over a longer time period. The shape of the release profile (percentage of drug released versus time) after the burst can be controlled to be linear or logarithmic or some more complex shape, again depending on the composition of the polymers comprising the device body and bioactive agent in the polymer.

Once a therapeutic range has been determined (for example, by a physician), the inventive polymer systems can be adjusted to provide the bioactive agent at a dosage that is within the therapeutic range. The inventive compositions provide improved means to control release of the bioactive agent, thus providing enhanced ability to deliver bioactive agent at desired rates and amounts.

In some aspects, the inventive biodegradable, implantable devices are fabricated of polymeric materials that can limit initial release of bioactive agent and provide control over the shape of the release profile curves.

The invention will now be described with reference to the following non-limiting examples.

### EXAMPLE 1

An amphiphilic copolymer of polyethylene glycol terephthalate (PEGT, Mw=300 g/mol) and polybutylene terephthalate (PBT), wherein the weight ratio of PEGT to PBT was 55 to 45 was obtained from OctoPlus BV, Bilthoven, The Netherlands. The copolymer in an amount of 1.2089 grams was dissolved in 20 milliliters of dichloromethane to make an approximately 60 milligram per milliliter solution. This solution was of a suitable viscosity for dip coating.

A glass stirring rod of approximately 5 millimeter diameter was cleaned with dichloromethane and permitted to dry. The cleaned, dried rod was then repeatedly dipped into the copolymer solution. The rod was dipped into the solution for 10 seconds (total immersion and removal time from solution), followed by a period of drying at room temperature for 60 seconds. A total of 11 dip cycles (10 second dwell, 60 second dry) were used and resulted in a whitish, opaque coating.

The resulting copolymer coating was dried overnight in a room temperature fume hood to remove any residual solvent. The rod and coating were then soaked in deionized water for 130 minutes to facilitate removal of the copolymer coating from the glass rod. The coating was removed from the glass rod by twisting and pulling the rod with a gloved hand.

The thickness of the resulting 5 centimeter long tube was 0.17 millimeter at the bottom and 0.05 millimeter at the top. The resulting copolymer tube was stored at room temperature.

### EXAMPLE 2

Biodegradable stents including bioactive agent are prepared as follows. An amphiphilic copolymer of polyethylene glycol terephthalate (PEGT, Mw=300 g/mol) and polybutylene terephthalate (PBT), wherein the weight ratio of PEGT to PBT was 55 to 45 as described in Example 1 is obtained from OctoPlus BV, Bilthoven, The Netherlands. The copolymer is dissolved in dichloromethane. Once dissolved, bioactive agent is then added to the solution in a polymer/drug weight ratio as desired. The solution is stirred until it becomes homogeneous, and the viscosity is adjusted to achieve an appropriate level for dip coating.

A glass stirring rod of approximately 5 millimeter diameter is cleaned and permitted to dry as described in Example 1. The cleaned, dried rod is then repeatedly dipped into the copolymer solution as described in Example 1 for a desired number of dip cycles.

The resulting copolymer coating is dried overnight in a room temperature fume hood to remove any residual solvent. The copolymer containing bioactive agent coating is then removed from the glass rod as described in Example 1.

Bioactive Agent Elution

Dried devices are weighed prior to elution experiments to determine an initial (dry) weight. Any suitable Elution Assay can be used to determine the extent and/or rate of bioactive agent release from the devices under physiological conditions. In one illustrative Elution Assay, bioactive agent release is measured in phosphate-buffered saline (PBS, pH 7.4). In a typical procedure, each device is placed in a 7 milliliter amber scintillation vial. A magnetic stir bar and 4 milliliters of PBS buffer (1 liter water, 9 grams sodium chloride, 0.27 grams potassium phosphate monobasic (KH₂PO₄), and 1.4 grams potassium phosphate dibasic (K₂HPO₄)) is added to each of the vials. The vials are placed in a 37° C. water bath. At each sampling time (usually 4 or 5 times on the first day followed by daily sampling thereafter), the device is removed and placed in fresh buffer solution in a new vial. Sampling times can be chosen based upon the expected or desired elution rate. At the sampling time point, the device is removed from the vial and placed into a new vial containing fresh PBS. Concentration of bioactive agent is determined in the spent buffer by UV spectroscopy using the characteristic wavelength for each bioactive agent. This concentration can be converted to a mass of bioactive agent released from the copolymer using molar absorptivities. The cumulative mass of the released bioactive agent is calculated by adding the individual sample mass after each removal. The release profile is obtained by plotting the amount of released bioactive agent as a fuinction of time.

Once the elution experiment is finished, any remaining portion of the device is dried overnight in a vacuum oven set at room temperature (25-27° C.) and weighed to ensure the accuracy of the UV spectroscopy results.

Compression Resistance (Wet)

Compression resistance tests can be conducted on an Instron test machine or other similar force gauge (such as an RX500, provided by Machine Solutions Inc.). For testing, a stent is placed in appropriate simulated body fluids, blood, or saline and maintained at 37° C. during evaluation.

To determine an initial yield force of the stent, the stent is expanded to nominal stent diameter. The stents are then placed on a flat surface in a compression tester (Instron). The flat plate of the Instron is lowered onto the stent, thus compressing the stent radially. The raw data of plate distance traveled versus force can be measured and plotted to obtain the constrained diameter versus force curve of the stent specimen.

To simulate conditions of use, the stent can be subjected to two cycles of the following three sequential steps. First, the stent can be compressed to a specified outer diameter (OD) at a controlled speed. This portion of the test characterizes the compression resistance of the stent. Second, the stent can be held in the compressed state for a given duration, typically one minute. This portion of the test characterizes the force decay or loss of recovery force. Third, the constraint on the stent is relaxed at a controlled rate. This portion of the test characterizes the self-expansion force of the stent.

Flexibility (Bending Force)

Flexibility tests can be conducted using a force gauge using a mandrel at the opposite end of the stent. For testing, a stent is placed in simulated body fluids, blood, or saline at 37° C.

The stent is compressed to an outer diameter (OD) according to the particular use (for example, 6-7 mm for uretal stents, 2-5 mm for vascular stents). The compressed stent is mounted on a mandrel of a size approximately equal to the inner diameter of the stent. The mandrel is held in place during testing. A force gauge (Instron) with a small contact area is positioned at the opposite end of the stent. The force gauge is lowered at a controlled rate and the force versus distance traveled is measured. The values can be plotted to establish a bend distance versus force curve.

Similarly, the flexibility of an expanded stent can be determined utilizing the above protocol, but eliminating the initial step of compressing the stent prior to mounting on a mandrel for testing. Again, the mandrel is held in place while a force gauge is lowered at a controlled rate on the opposite stent end. The force versus bend distance can be plotted to establish a bend distance versus force curve.

Swellability in Water

To determine the swellability of the stent in fluids, a formed stent is first weighed dry to determine an initial (dry) weight. The stent is then immersed in saline or water at 37° C. for 24 hours. After this period, the stent is removed and reweighed. The water uptake is equal to the following:
(wet weight−dry weight)/dry weight.
Degradation Studies

To study the degradation of stents according to embodiments of the invention, formed stents are weighed to establish an initial weight. PBS (pH 7.4) is pipetted into a vial with a Teflon™lined cap. The stents are immersed into the PBS. A stir bar is placed into the vial and the cap is screwed tightly onto the vial. The PBS is stirred with the use of a stir plate, and the temperature of the PBS is maintained at 37° C. with the use of a water bath. The sampling times are chosen based upon the expected or desired degradation rate. At the sampling time point, the stents are removed from the PBS, washed with water, dried, and weighed. Change in stent weight over time can be plotted to monitor degradation rate of the stent.

Biocompatibility Studies

To study biocompatibility of stents according to embodiments of the invention, formed stents can be implanted into any suitable animal model (such as porcine or the like). Response to the stents, such as inflammatory response, thrombus formation, immune response, adventitial damage, clinically significant neointimal formation, and the like can be monitored. Appropriate standards can be utilized to determine response attributable to the biodegradable materials of the inventive devices.

In some aspects, the inventive biodegradable devices demonstrate excellent uniformity and durability during use. Device body uniformity and durability can be observed and assessed as follows.

One aspect of device uniformity relates to surface features of the device body. The inventive devices can be examined for uniformity and defects using a Field Emission Scanning Electron Microscope (SEM) at a low beam voltage (1 kV) which allows detailed imaging of surface features. Illustrative surface defects can include areas of delamination or cracking of the polymer material, surface areas that lack one or more coated layers of the polymer (when applied in successive layers as described in Example 1), and the like. An overall survey of the device body quality is made at low magnification, and when features of interest are identified, higher magnification images are taken. From the overall survey, a qualitative ranking of the relative amount and type of defects in the device surface can be made.

Another aspect of device uniformity relates to the uniformity of mixing of bioactive agent into the biodegradable compositions. This aspect of the device body can be imaged using a confocal scanning Raman microscope. Laser light (532 nm wavelength) is focused onto the device body via a 100× microscope objective (numerical aperture 0.95), and the device body is scanned in three directions using a piezoelectric transducer driven platter. The scattered light from the device body is collected by the microscope, filtered, split into its spectrum using a spectrograph, and detected with a CCD detector. Thus, for each position (pixel) in the image, a Raman spectrum is measured. Reference spectra of the pure bioactive agent and pure polymer are incorporated into an augmented classical least squares analysis to create separate images of bioactive agent only and polymer only. These images are overlapped to create a composite color coded image of the distribution of bioactive agent within the biodegradable polymer.

Uniformity of bioactive agent distribution within the polymer material can impact the release profile of the bioactive agent. If a large percentage of the bioactive agent is concentrated at a particular portion of the polymer material (device body), the release of the bioactive agent is less likely to exhibit controlled release kinetics. For example, if a large percentage of bioactive agent is concentrated at the surface of a device body, the bioactive agent is more likely to be released quickly from the device, since the bioactive agent does not have a large diffusion distance to the surface. In contrast, a bioactive agent that is concentrated towards the device body interior may have a larger diffusion distance to travel, and thus release of the bioactive agent may be delayed relative to the prior exemplary device. Moreover, concentration of a bioactive agent within a polymer can result in a release profile that includes one or more sudden increases in release, as polymer degradation reaches the area of bioactive agent concentration.

As used herein, the term “durability” refers to the ability of a polymer material to maintain integrity when subjected to forces typically encountered during use (for example, normal force, shear force, and the like). A more durable device body is less easily mechanically compromised by abrasion or compression. Durability of a device body can be assessed by subjecting the device to conditions that simulate use conditions. For example, to simulate use of the biodegradable polymeric devices, the devices (stents) are placed over sample angioplasty balloons. The stent is then crimped onto the balloon using a laboratory test crimper (available from Machine Solutions, Brooklyn, N.Y.). The stent and balloon are then placed in a water bath having a temperature of 37° C. After 5 minutes of soaking, the balloon is expanded using air at 5 atmospheres (3800 torr) of pressure. The balloon is then deflated, and the stent is removed. The stent is then examined by optical and scanning electron microscopy to determine the amount of damage caused by cracking and/or delamination of the polymer at the device surface. Devices with extensive damage are considered unacceptable for a commercial medical device. Testing can be followed up with contact angle testing, and/or SEM analysis to visualize the polymer material integrity.

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following claims. All patents, patent documents, and publications cited herein are hereby incorporated by reference as if individually incorporated.

We claim:

1. An implantable intraluminal medical device comprising a body member fabricated of a biodegradable amphiphilic block copolymer comprising hydrophilic blocks and hydrophobic blocks.

2. The medical device according to claim 1 wherein the body member comprises an intravascular medical device.

3. The medical device according to claim 2 wherein the intravascular medical device is selected from stents, stent grafts, shunts, anastamosis devices, occlusion devices, septal defect treatment devices, and closure devices.

4. The medical device according to claim 1 wherein the body member is configured for extravascular placement within a patient.

5. The medical device according to claim 4 wherein the body member is configured for placement within the brain, gastrointestinal, duodenum, biliary ducts, esophagus, urethra, lymphatic vessels, reproductive tracts, trachea, respiratory ducts, and otological passages.

6. The medical device according to claim 1 wherein the hydrophilic blocks comprise polyalkylene glycol.

7. The medical device according to claim 6 wherein the polyalkylene glycol is selected from the group polyethylene glycol, polypropylene glycol, and polybutylene glycol.

8. The medical device according to claim 7 wherein the polyalkylene glycol is selected from the group polyethylene glycol terephthalate, polypropylene glycol terephthalate, and polybutylene glycol terephthalate.

9. The medical device according to claim 6 wherein the polyalkylene glycol blocks comprise polymers having a formula:
—OLO—CO—R—CO—
wherein L is a divalent organic radical remaining after removal of terminal hydroxyl groups from a poly(oxyalkylene)glycol, O represents oxygen, C represents carbon, and R is a substituted or unsubstituted divalent radical remaining after removal of carboxyl groups from a dicarboxylic acid.

10. The medical device according to claim 6 wherein the hydrophobic blocks comprise aromatic polyester formed from an alkylene glycol having 2 to 8 carbon atoms and a dicarboxylic acid.

11. The medical device according to claim 10 wherein the polyester is selected from the group polyethylene terephthalate, polypropylene terephthalate, and polybutylene terephthalate.

12. The medical device according to claim 10 wherein the aromatic polyester blocks comprise polymers having a formula:
—OEO—CO—R—CO—
wherein E is an organic radical selected from the group of substituted or unsubstituted alkylene radical shaving 2 to 8 carbon atoms, and a substituted or unsubstituted ether moiety, O represents oxygen, C represents carbon, and R is a substituted or unsubstituted divalent aromatic radical.

13. The medical device according to claim 1 wherein the amphiphilic block copolymer comprises polyethylene glycol/polybutylene terephthalate block copolymer.

14. The medical device according to claim 1 wherein the amphiphilic block copolymer includes one or more bioactive agents.

15. The medical device according to claim 14 wherein the bioactive agent is selected from antiproliferative agents, anti-inflammatory agents, inhibitors of angiogenesis, hormonal agents, or a combination of any two or more of these.

16. The medical device according to claim 15 wherein the antiproliferative agent is selected from taxol, sirolimus (rapamycin), analogues of rapamycin (“rapalogs”), tacrolimus, ABT-578 from Abbott, everolimus, paclitaxel, taxane, vinorelbine.

17. The medical device according to claim 15 wherein the anti-inflammatory agent is selected from hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide.

18. The medical device according to claim 15 wherein the inhibitor of angiogensis is selected from angiostatin, anecortave acetate, thrombospondin, anti-VEGF antibody such as anti-VEGF fragment.

19. The medical device according to claim 15 wherein the hormonal agent is selected from estrogen, estradiol, progesterol, progesterone, insulin, calcitonin, parathyroid hormone, peptide and vasopressin hypothalamus releasing factor.

20. The medical device according to claim 1 wherein the body member has a minimum compression resistance of 5 Newtons.

21. The medical device according to claim 1 wherein the body member has a minimum tensile strength of 500 psi.

22. The medical device according to claim 1 wherein the body member has a minimum tensile modulus of 6000 psi.

23. The medical device according to claim 1 further comprising a coating on a surface of the body member.

24. The medical device according to claim 23 wherein the coating is provided on a portion of the body member surface.

25. The medical device according to claim 23 wherein the coating comprises a biodegradable polymer selected from an amphiphilic copolymer having hydrophilic blocks and hydrophobic blocks, polylactic acid, copolymers of polylactic acid with glycolic acid, and polycarbonates.

26. The medical device according to claim 1 further comprising a sheath.

27. The medical device according to claim 1 further comprising microparticles.

28. The medical device according to claim 1 further comprising one or more nondegradable fibers.

29. The medical device according to claim 14 configured to release bioactive agent for a period of two weeks or more.

30. The medical device according to claim 29 configured to release bioactive agent for a period of four weeks or more.

31. A method of making a device for the controlled release of bioactive agent, the method comprising steps of providing a biodegradable amphiphilic block copolymer comprising hydrophilic blocks and hydrophobic blocks, and forming the copolymer into an implantable intraluminal medical device.

32. The method according to claim 31 wherein the step of forming the copolymer into an implantable intraluminal medical device is accomplished by dip coating a substrate in the copolymer solution.

33. A method for delivery of bioactive agent to a patient in a controlled manner, the method comprising steps of providing an implantable intraluminal device to a patient, the device comprising a body member fabricated of a polymer matrix comprising one or more bioactive agents and a biodegradable amphiphilic block copolymer comprising hydrophilic blocks and hydrophobic blocks.

34. The method according to claim 33 further comprising a step of allowing the device to remain in the patient for a selected period of time, wherein the device is configured to degrade upon implantation for a degradation period, and wherein bioactive agent is released in a controlled manner for a bioactive agent release period, the release period constituting at least a portion of the degradation period.

35. The method according to claim 34 wherein release period comprises 50% or less of the degradation period.

36. The method according to claim 34 wherein the degradation period is in the range of 0.5 to 2 years.
